(19) United States
(12) Patent Application Publication
(10) Pub. No.: US 2025/0268980 A1
(43) Pub. Date: Aug. 28, 2025

(54) AN ANTICANCER THERAPY TARGETING RAS AND OTHER ONCOGENIC SIGNALS BY INHIBITION OF DYNAMIC BLEBBING AND/OR SEPTINS

(71) Applicant: THE BOARD OF REGENTS OF THE UNIVERSITY OF TEXAS SYSTEM, Austin, TX (US)

(72) Inventors: Andrew WEEMS, Carrollton, TX (US); Gaudenz DANUSER, Dallas, TX (US); Erik WELF, Dobbs Ferry, NY (US); Meghan DRISCOLL, Dallas, TX (US)

(73) Assignee: THE BOARD OF REGENTS OF THE UNIVERSITY OF TEXAS SYSTEM, Austin, TX (US)

(21) Appl. No.: 18/858,042

(22) PCT Filed: Apr. 21, 2023
(86) PCT No.: PCT/US2023/066073

§ 371 Date: Oct. 18, 2024

Related U.S. Application Data
(60) Provisional application No. 63/334,029, filed on Apr. 22, 2022.

(51) Int. Cl. A61K 38/16

(52) U.S. Cl. CPC A61K 38/168

(57) ABSTRACT

The present disclosure generally relates to compositions and methods for targeting pro-survival signaling hubs in anoikis-resistant cells, such as anoikis-resistant cancer cells. Embodiments also include methods of changing morphology of cells by inhibiting bleb formation which, in part, reduces survival of the cell and proliferation of the cell by disrupting downstream bleb-driven signaling.

### CROSS REFERENCE TO RELATED APPLICATIONS

This application claims priority to U.S. Provisional Patent Application Ser. No. 63/334,029, filed Apr. 22, 2022, the contents of which is hereby incorporated by reference in its entirety.

### ACKNOWLEDGEMENT OF GOVERNMENT SUPPORT

This invention was made with government support under Grant No. GM136428 awarded by the National Institutes of Health. The government has certain rights in this invention.

### INCORPORATION BY REFERENCE OF SEQUENCE LISTING

This application contains a sequence listing that has been submitted via PatentCenter in a computer readable format and is hereby incorporated by reference in its entirety. The computer readable file, created on Apr. 21, 2023, is named 106546-755474 (UTSD4039) _SequenceListing.xml and is about 25,400 bytes in size

### BACKGROUND

### 1. Field

The present inventive concept is directed to compositions and methods of targeting pro-survival signaling hubs in anoikis-resistant cells, such as anoikis-resistant cancer cells.

### 2. Discussion of Related Art

Despite recent therapeutic advances in cancer treatments, metastasis remains the principal cause of cancer death. As an example, metastatic melanoma is associated with poor prognoses and limited therapeutic options. About 50-60% of melanomas carry the BRAF (V600E) activating mutation, an oncogene that drives cell proliferation and survival through hyperactivation of the MAPK pathway. The past decade has seen the development of several therapeutics that target this oncogenic signaling through MAPK inhibition (MAPKi), but though >80% of BRAF mutant patients respond well to treatment initially, therapeutic resistance is developed in most within 6-12 months. For patients carrying NRAS mutations the situation is more dire, as no targeted therapies yet exist against this genotype. Newer immunotherapies seem to generate durable results, but their impact is limited by a<40% response rate in patients, at least 25% of whom will go on to experience drug-resistant relapse. As such, there is a need in the field for understanding of cellular regulators of cell motility and metastasis, drug resistant melanoma, and developing therapies capable of treating cancer, including metastatic cancer.

### SUMMARY OF THE INVENTION

The present disclosure is based, at least in part, on identification of a novel morphology-driven survival signaling pathway in cells with septins bridging blebbing with downstream survival signaling. Because septins serve as the single node bridging blebbing with downstream survival signaling, targeting this lynchpin of bleb signaling can be a therapeutic strategy.

The present disclosure provides compositions and methods for treating a cancer in a subject in need thereof. In certain embodiments, methods of treating a cancer in a subject may comprise administering to a subject in need thereof at least one septin inhibitor, wherein the subject has or is suspected of having at least one malignant solid tumor. In some aspects, at least one malignant solid tumor may comprise a sarcoma, a carcinoma, a lymphoma, or any combination thereof. In some other aspects, at least one malignant solid tumor may comprise a testicular tumor, ovarian tumor, cervical tumor, kidney tumor, bladder tumor, head-and-neck tumor, liver tumor, stomach tumor, lung tumor, endometrial tumor, esophageal tumor, breast tumor, central nervous system tumor, germ cell tumor, prostate tumor, melanoma, colon tumor, mesothelioma, osteogenic sarcoma, or any combination thereof. In some aspects, at least one malignant solid tumor can be a metastatic solid tumor.

In some embodiments, methods for treating a cancer disclosed herein may comprise administering to a subject at least one pan septin inhibitor. In some aspects, at least one pan septin inhibitor may comprise forchlorfenuron or an analog thereof. In some embodiments, methods for treating a cancer disclosed herein may comprise administering to a subject at least one septin inhibitor wherein the septin inhibitor may comprise at least one of a peptide, an antibody, a chemical, a compound, an oligo, a nucleic acid molecule, or any combination thereof.

In some embodiments, methods for treating a cancer disclosed herein may comprise treating malignant solid tumor cells having a higher rate of cell death in the subject compared to an untreated subject with identical disease condition and predicted outcome. In some aspects, methods disclosed herein may be used when the risk of tumor metastasis is decreased in the subject compared to an untreated subject with identical disease condition and predicted outcome.

In certain embodiments, the present disclosure provides for compositions and methods of treating tumors in a subject. In some embodiments, methods of treating a tumor as disclosed herein may comprise administering to a subject in need thereof at least one septin inhibitor; wherein the subject is undergoing or will undergo an anti-cancer therapy comprising one or more targeted therapies; and sensitizing the tumor to the one or more targeted therapies in the subject. In some aspects, a tumor to be treated by the methods disclosed herein may be resistant to the one or more targeted therapies. In some aspects, a tumor to be treated by the methods disclosed herein may comprise a sarcoma, a carcinoma, a lymphoma, or any combination thereof. In some aspects, a tumor to be treated by the methods disclosed herein may comprise a testicular tumor, ovarian tumor, cervical tumor, kidney tumor, bladder tumor, head-and-neck tumor, liver tumor, stomach tumor, lung tumor, endometrial tumor, esophageal tumor, breast tumor, central nervous system tumor, germ cell tumor, prostate tumor, colon tumor, melanoma, mesothelioma, osteogenic sarcoma, or any combination thereof. In some other aspects, a tumor to be treated by the methods disclosed herein may comprise a melanoma, a lung tumor, a colon tumor or any combination thereof. In some other aspects, a tumor to be treated by the methods disclosed herein may comprise a melanoma. In some aspects, a tumor to be treated by the methods disclosed herein may comprise a metastatic tumor. In still some other aspects, a tumor to be treated by the methods disclosed herein may comprise one or more somatic mutations in a BRAF gene, a NRAS gene, a RAS gene, a RAF gene, an EGFR gene, a KRAS gene or any combination thereof. In some other aspects, a tumor to be treated by the methods disclosed herein may be anoikis resistant.

In some embodiments, the one or more targeted therapies may comprise one or more targeted MAPK signaling pathway inhibitor (MAPKi) agents. In some embodiments, the one or more targeted MAPKi agents may comprise PD-325901, TAK-733, binimetinib, cobimetinib, selumetinib, trametinib, gefitinib, lapatinib, ARRY-614, ralimetinib, ulixertinib, erlotinib, bevacizumab, or any combination thereof. In some embodiments, the one or more targeted therapies herein may comprise one or more targeted BRAF inhibitor agents. In some embodiments, a subject to be treated according to the methods disclosed herein may be undergoing or will undergo an additional targeted anticancer therapies comprising one or more targeted BRAF inhibitor agents. In some aspects, the one or more targeted BRAF inhibitor agents may comprise vemurafenib, dabrafenib, encorafenib, PLX8394, CCT3833, LY3009120, lifirafenib, belvarafinib, TAK-580, RO5126766, trametiglue or any combination thereof.

In some embodiments, methods of treating a tumor as disclosed herein may comprise administering to a subject in need thereof at least one pan septin inhibitor. In some embodiments, methods of treating a tumor as disclosed herein may comprise administering to a subject in need thereof at least one of a septin1 inhibitor, a septin2 inhibitor, a septin3 inhibitor, a septin4 inhibitor, a septin5 inhibitor, a septin6 inhibitor, a septin7 inhibitor, a septin8 inhibitor, a septin9 inhibitor, a septin 10 inhibitor, a septin11 inhibitor, a septin 12 inhibitor, a septin14 inhibitor, or any combination thereof. In some aspects, the at least one septin inhibitor may comprise at least one of a peptide, an antibody, a chemical, a compound, an oligo, a nucleic acid molecule, or any combination thereof. In some other aspects, the at least one septin inhibitor may comprise forchlorfenuron or an analog thereof.

In certain embodiments, the present disclosure provides for methods of treating a tumor comprising administering to a subject in need thereof an effective amount of at least one septin inhibitor. In some embodiments, a subject to be treated according to the methods disclosed herein may have or be suspected of having a tumor comprising one or more somatic mutations in a BRAF gene, a NRAS gene, a RAS gene, a RAF gene, an EGFR gene, a KRAS gene or any combination thereof. In some aspects, the tumor comprises a melanoma, a lung tumor, a colon tumor or any combination thereof.

In some embodiments, methods of treating a tumor disclosed herein may comprise administering an effective amount of at least one septin inhibitor, wherein the effective amount of at least one septin inhibitor can reduce the invasiveness, metastasis, therapy resistance, relapse, or any combination thereof of the tumor compared to an untreated subject with identical disease condition and predicted outcome.

In some embodiments, a subject to be treated according to the methods disclosed herein may have undergone or be undergoing at least one other therapy for cancer. In some aspects, the at least one other therapy for cancer may comprise administration of PD-325901, TAK-733, binimetinib, cobimetinib, selumetinib, trametinib, vemurafenib, dabrafenib, encorafenib, or any combination thereof.

In some embodiments, a subject to be treated according to the methods disclosed herein may have or is suspected of having a MAPKi resistant melanoma. In some embodiments, a subject to be treated according to the methods disclosed herein may have or is suspected of having an anoikis resistant melanoma.

In certain embodiments, the present disclosure provides for methods comprising: (i) contacting a cancer cell with least one septin inhibitor, wherein the cancer cell is or suspected to be anoikis resistant; and (ii) inhibiting the anoikis resistance in the cancer cell after contacting the cancer cell with least one septin inhibitor. In some aspects, cell survival may be decreased after contacting the cancer cell with least one septin inhibitor. In some other aspects, cell proliferation may be decreased after contacting the cancer cell with least one septin inhibitor.

In certain embodiments, the present disclosure provides for methods of disrupting cell survival, cell proliferation, and/or cell differentiation. In some embodiments, methods of disrupting cell survival may comprise contacting the cell with least one septin inhibitor, wherein the at least one septin inhibitor disrupts survival, proliferation and/or differentiation by altering the ability of at least one bleb-induced septin structure to amplify or inhibit one or more downstream signaling pathways involved in cell survival, proliferation, differentiation, or any combination thereof. In some aspects, the one or more downstream signaling pathways that promote cell survival may comprise Notch, CD44, RAS, a receptor tyrosine kinase, or any combination thereof.

In some embodiments, the survival, proliferation, and/or differentiation of the cell according to the methods disclosed herein may be bleb-dependent. In some aspects, the cell may have or is suspected of having at least one bleb formation. In some aspects, the cells may comprise fibroblasts, endothelial cells, mesenchymal cells, cancer cells, immune cells, germ cells, stem cells, or any combination thereof. In some examples, the cell may comprise a cancer cell.

In certain embodiments, the present disclosure provides for methods of changing morphology of a cell. In some embodiments, methods of changing morphology of a cell may comprise contacting the cell with least one inhibitor of bleb formation, wherein the at least one inhibitor of bleb formation reduces survival of the cell, proliferation of the cell, and/or differentiation of the cell by disrupting downstream bleb-driven signaling, and wherein the morphology of the cell comprises fewer or smaller cell surface blebs upon contacting the cell with the at least one inhibitor of bleb formation compared to an untreated cell. In some aspects, the cells may comprise fibroblasts, endothelial cells, mesenchymal cells, cancer cells, immune cells, germ cells, stem cells, or any combination thereof. In some examples, the cell may comprise a cancer cell.

The foregoing is intended to be illustrative and is not meant in a limiting sense. Many features and subcombinations of the present inventive concept may be made and will be readily evident upon a study of the following specification and accompanying drawings comprising a part thereof. These features and subcombinations may be employed without reference to other features and subcombinations.

### BRIEF DESCRIPTION OF THE DRAWINGS

Embodiments of the present inventive concept are illustrated by way of example in which like reference numerals indicate similar elements and in which:

FIGS. 1A-1J depict graphs and images illustrating disruption of anoikis resistance in melanoma cells by bleb inhibition. FIG. 1A shows a graph demonstrating the fraction of cell surface comprised of blebs for MV3 cells treated with different concentrations of WGA. Dashed lines separate quartiles. Dots represent individual cells. FIGS. 1B-1F show graphs demonstrating cell death as a function of WGA treatment at different doses for adhered and detached melanoma cells. Cells grown for 24 hours and assayed for cell death using ethidium homodimer staining. All treatment groups grown and assayed in simultaneous paired experiments, which were performed three times for each cell line (See FIGS. 6A-6L for individual experiments). Replicate data were normalized by subtracting negative control values from each treatment group. Error bars represent 95% confidence intervals. Sum cell counts for all replicates in ascending order of WGA dosage (see Table 1 for individual counts) where FIG. 1B shows MV3 Det. (470, 602, 479, 501, 562), MV3 Adh. (688, 569, 597, 426, 544); FIG. 1C shows M498 Det. (290, 291, 159, 187, 133), M498 Adh. (405, 378, 347, 285, 316); FIG. 1D shows unstressed A375 Det. (867, 797, 670, 847, 679), Unstressed A375 Adh. (576, 453, 589, 578, 555); FIG. 1E shows prestressed A375 Det. (496, 493, 649, 317, 325), Prestressed A375 Adh. (495, 581, 448, 504, 475); and FIG. 1F shows A375 Adh+MAPKi, A375 Det.+MAPKi. FIG. 1G shows images demonstrating cell surface renderings of representative cells showing mean intracellular curvature. FIGS. 1H-11 show graphs demonstrating cell death upon bleb inhibition using VitroGel coffins for adhered and detached melanoma cells (FIG. 1H, MV3 cells; FIG. 11, A375 cells). Cells were grown for 24 hours in either integrin-binding VitroGel-RGD (adhered) or non-integrin-binding VitroGel (detached) and assayed for cell death using ethidium homodimer staining. Dots represent individual experiments. Sum cell counts for all replicates as follows, listed as they appear in the figure from left to right (see Table 2 for individual counts): 367, 389, 461, 509. FIG. 1J shows morphology of cells in “adhered” conditions, relating to FIGS. 1B-1F. Cells grown without perturbation for 24 h on adherent chamber slides. Representative of either 688 (MV3), 405 (M498), or 576 (A375) cells observed

FIGS. 2A-2O depict graphs and images illustrating bleb-generated plasma membrane curvature driving the assembly of cortical septin structures. FIG. 2A shows images of surface renderings of an MV3 cell demonstrating either mean local intensity of septin signal or mean local intracellular curvature. Septin signal was murine SEPT6-GFP. Cells were embedded in soft bovine collagen. FIG. 2B shows a graph demonstrating the directional correlation between blebs and septin localization in 17 MV3 cells. Cumulative correlative distribution is shown as orange solid line, randomized bleb localization control is shown as a dashed blue line, and zero correlation is shown as black dotted line. FIG. 2C shows a graph demonstrating the local mean intensity of septin signal as a function of local intracellular curvature, using same cells. The orange line indicates curvature/intensity relationship on the surface of blebs, the blue line on non-bleb surfaces, and the dashed purple line on the entirety of surfaces. Error bands represent 95% confidence intervals. FIG. 2D shows a graph demonstrating the probability distributions of mean local positive intracellular curvature for all cells in FIG. 1A. FIG. 2E shows a graph demonstrating the local mean septin intensity and intracellular curvature as a function of distance from bleb edges using same cells as FIG. 2B. FIG. 2F shows representative images demonstrating septin localization in MV3 melanoma cells with diverse perturbations of bleb formation. Representative cells received treatment as follows: WGA (50 ug/ml), VitroGel, H1152 (1 μM), NSC668394 (10 μM). Maximum intensity projections and single optical slices of 0.16 micron thickness. Septin signal murine SEPT6-GFP. Arrowheads indicate septin accumulation in perturbed cells in regions with residual high-curvature. Cells embedded in soft bovine collagen. FIG. 2G shows a graph demonstrating the fraction of cortical voxels (within 0.96 μm of surface) in basal and bleb-inhibited MV3 cells with septin intensity higher than cytoplasmic mean intensity. Dashed lines separate quartiles. Basal/VitroGel tested with two sample T-test using pooled variance (p=0.013), normality tested with Shapiro-Wilk (p=0.2579 & 0.31), variance tested with two-tailed F test (p=0.448). Basal/WGA tested with Welch's T-test (p<0.0001), normality tested with Shapiro-Wilk (p=0.4539 & 0.7941), variance tested with two-tailed F-test (p=0.0114). FIGS. 2H-21 show graphs demonstrating the same data as FIG. 2G expressed as a function of cell surface fraction possessing intracellular curvature either above (FIG. 2H, K>0.4 μm 1) or below (FIG. 21, 0>K>0.4 μm⁻¹) septin recruiting threshold. FIG. 2J shows representative images demonstrating septin localization in a representative MV3 melanoma cell expressing septin curvature-sensing mutant SEPT6 (AAH)-GFP. FIG. 2K shows representative images demonstrating septin localization in a representative MV3 melanoma cell expressing septin polymerization mutant SEPT2 (33-306). FIG. 2L shows representative images of a time-lapse showing septin accumulation during an individual blebbing event. Single optical section of 0.16 μm thickness. Septin signal was murine SEPT6-GFP. Cell was embedded in soft bovine collagen. FIG. 2M shows representative images of a time-lapse showing changes in gross septin localization over 14 minutes in maximum intensity projections of a representative MV3 cell. Individual timepoints shown in grayscale on left and indicated pseudo-color in composite on right. Septin signal was murine SEPT6-GFP. Cell was embedded in soft bovine collagen. FIG. 2N shows SEPT6-GFP intensity as function of distance from bleb edges in cells expressing SEPT2 (33-306), overlaying SEPT6-GFP intensity from FIG. 2E. FIG. 2O shows temporal cross-correlation between SEPT6-GFP intensity and intracellular mean curvature for positive and negative contigs. Cell imaged for 4 minutes, stack acquisition=0.83 Hz.

FIGS. 3A-3F depict graphs and images illustrating that septins are necessary for bleb-dependent anoikis resistance. FIG. 3A shows representative images of septin localization in different melanoma cell lines. Maximum intensity projections (above) and single optical slices of 0.16 micron thickness (below) are shown for representative cells. Septin signal was murine SEPT6-GFP. Cells were embedded in soft bovine collagen. FIGS. 3B-3F show graphs demonstrating cell death upon septin inhibition with 50 μM FCF in adhered and detached melanoma cells. Cells were grown as in described in FIG. 1 and treated with either FCF or EtOH control for 24 hours. Data were normalized by subtracting paired negative control values from each treatment group. Dots represent individual experiments. Sum cell counts for all replicates listed as they appear in the figure from left to right, with control counts in parentheses (see Table 4 for individual counts) where: FIG. 3B: MV3 Adh. 440 (470), MV3 Det. 644 (688); FIG. 3C: M498 Adh. 298 (290), M498 Det. 338 (405); FIG. 3D: unstressed A375 Adh. 823 (867), unstressed A375 Det. 562 (576); FIG. 3E: prestressed A375 Adh. 418 (541), prestressed A375 Det. 169 (205); and FIG. 3F shows A375 Adh+MAPKi, A375 Det.+MAPKi.

FIGS. 4A-4J depict graphs and images illustrating that septins scaffold NRAS, promoting NRAS/MAPK and NRAS/PI3K survival signaling. FIG. 4A shows a Spearman Correlation between NRAS and septin signal distributions on the surfaces of MV3 melanoma cells, compared to septin vs collagen negative control. White datapoints represent cells whose correlations were not significantly higher than analyses performed on the same surfaces with one signal randomly scrambled. Septin and NRAS signal distributions from the median cell in the dataset shown on the right. FIG. 4B shows a graph of the observed NRAS-GFP % enrichment at the cortex (voxels within 0.96 μm of surface) of individual unperturbed and septin-inhibited MV3 cells. Basal/FCF tested with Welch's T-test (p<0.0001), normality tested with Shapiro-Wilk (p=0.2191 & 0.2198), variance tested with two-tailed F test (p=0.0054). Basal/SEPT2 (33-306) was tested with two sample T-test using pooled variance (p=0.0101), normality was tested with Shapiro-Wilk (p=0.2191 & 0.7246), variance was tested with two-tailed F test (p=0.768). Dashed lines separate quartiles. FIG. 4C shows a graph depicting the Earth Mover's Distance (EMD) between observed NRAS-GFP and homogenous distribution of equivalent signal as measured for each unperturbed or septin-inhibited MV3 cell. Control/FCF was tested with Mann-Whitney U test (p=0.0007), normality was tested with Shapiro-Wilk (p=0.0198 & 0.9537). Control/SEPT2 (33-306) was tested with Mann-Whitney U test (p=0.0213), normality was tested with Shapiro-Wilk (p=0.0198 & 0.0265). FIG. 4D shows a graph demonstrating cell death upon expression of dominant negative NRAS (S17N) for adhered and detached MV3 cells. Cells were grown as described in FIG. 1 for 24 hours. Data were normalized by subtracting paired negative control values from each treatment group. Dots represent individual experiments. Sum cell counts for all replicates, listed as they appear in the figure from left to right, with control counts in parentheses (see Table 5 for individual counts): MV3 Adh. 1105 (1039), MV3 Det. 799 (734). FIG. 4E shows a graph demonstrating the effect of septin inhibition upon ERK activation levels in adhered and detached MV3 cells. Percent change in ERK activation between unperturbed and septin inhibited MV3 cells was measured by ERK-nKTR-GFP biosensor. Cells were grown as described in FIG. 1 for 12 hours. Dots represent individual experiments. Sum cell counts for all replicates as follows (see Table 6 for individual counts): Adh. Control 234, Adh. FCF 236, Det. Control 405, Det. FCF 339. FIG. 4F shows a graph demonstrating the effect of detachment upon ERK activation levels in unperturbed, septin-inhibited, and bleb-inhibited MV3 cells. Percent change in ERK activation between attached and detached MV3 cells was measured by ERK-nKTR-GFP biosensor. Cells were grown as described in FIG. 1 for 12 hours. Dots represent individual experiments. Sum cell counts for all replicates as follows (see Table 7 for individual counts): Adh. Control 276, Det. Control 294, Adh. SEPT2 (33-306) 274, Det. SEPT2 (33-306) 232, Adh. VitroGel 240, Det. VitroGel 205. FIG. 4G shows a Spearman Correlation between NRAS/Akt-PH and Septin/Akt-PH signal distributions on the surfaces of MV3 melanoma cells, compared to septin vs collagen negative control. White datapoints represent cells whose correlations were not significantly higher than analyses performed on the same surfaces with one signal randomly scrambled. Septin and Akt-PH signal distributions from the median cell in the dataset shown above. FIG. 4H shows the effect of septin or bleb inhibition upon PI3K activity in individual MV3 cells. PI3K activity was measured with the PI3K biosensor Akt-PH-GFP and expressed as fraction of pixels brighter than cytosolic intensity in normalized sum intensity projections. Median cells from all groups are shown on the right. FIG. 4I depicts representative images illustrating colocalization between NRAS and areas of increased PI3K activity. Representative micrographs shown are either maximum intensity projections or single slices of 0.16 micron thickness of the same MV3 cell. Top shows NRAS-GFP localization, bottom shows PI3K activity biosensor AktPH-SNAP-TMR localization. FIG. 4J shows observed NRAS-GFP percent enrichment at the cortex (voxels within 0.96 μm of surface) of individual MV3 cells with and without expression of SEPT6-HALO using the same pLVX-IRES-Hyg vector as the SEPT2 (33-306) construct. Included as a negative control demonstrating that the NRAS mislocalization in SEPT2 (33-306)—expressing cells is not due to effects arising from the protein expression construct. Significance tested with two sample one-sided T-test with pooled variance (p=0.738), normality tested with Shapiro-Wilk (p=0.315 & 0.426), variance tested with two-tailed F test (p=0.181). Dashed lines separate quartiles.

FIGS. 5A-5C depict graphs and images illustrating that disrupting bleb attenuation yields anoikis resistance to non-cancerous fibroblasts. FIG. 5A shows images of recently detached MEF cells expressing SEPT6-GFP, imaged either before or after bleb attenuation. Maximum intensity projections (above) and single optical slices of 0.16 micron thickness (below) are shown for representative cells. Cells were embedded in soft bovine collagen. FIG. 5B shows a fraction of MEF cells showing blebby morphologies 90, 120, and 180 minutes after detachment from substrate. Orange datapoints indicate uninhibited MEF cells, while green datapoints indicate endocytosis-inhibited MEFs expressing DYN2 (K44A) in paired, same-day experiments (solid or dashed lines indicate paired data). Representative cells after 180 minutes of detachment are shown to the right. FIG. 5C shows a graph demonstrating additional caspase activity after 4 hours in detached MEF cells compared to paired adhered cells. Orange datapoints indicate uninhibited MEF cells, while green datapoints indicate endocytosis-inhibited MEFs expressing DYN2 (K44A). Red datapoints indicate DYN2 (K44A)—expressing cells treated with 10 μg/mL WGA and purple indicates DYN2 (K44A)—expressing cells treated with 50 μM FCF. Caspase activity was measured using CellEvent Caspase-3/7 biosensor.

FIGS. 6A-6L depict graphs illustrating individual bleb inhibition in viability experiments. All paired adhered/detached experiments shown were performed on the same day and seeded with melanoma cells from the same diluted cell suspensions to reflect cell death as a function of WGA treatment at different doses for adhered and detached melanoma cells. FIGS. 6A-6C show cell death in individual replicates of adhered and detached MV3 cells treated with increasing doses of WGA as indicated. FIGS. 6D-6F show cell death in individual replicates of adhered and detached M498 cells treated with increasing doses of WGA as indicated. FIGS. 6G-6l show cell death in individual replicates of adhered and detached unstressed A375 cells treated with increasing doses of WGA as indicated. FIGS. 6J-6L show cell death in individual replicates of adhered and detached pre-stressed A375 cells treated with increasing doses of WGA as indicated.

FIG. 7 depicts representative images illustrating stable septin structures emerging when pulses occurred in close proximity. Images are representative frames captured from high-speed time-lapse data.

FIG. 8A-8C provides a graphical illustration and data supporting that a SEPT6 (AAH)-GFP mutation disrupts curvature sensing blocks septin localization to the cell surface. FIG. 8A is a cartoon illustrating the mechanism underlying the altered function of the SEPT6 (AAH)-GFP mutant. FIG. 8B depicts mean local SEPT6 (AAH)-GFP intensity and intracellular mean curvature as a function of distance from bleb edges, calculated from 630 blebs across 5 MV3 cells. Error bands indicate 95% confidence intervals. FIG. 8C depicts difference in mean local septin intensity compared to local maxima as a function of distance from bleb edge for both SEPT6-GFP (as seen in FIG. 2E) and SEPT6 (AAH)-GFP.

FIG. 9 depicts a schematic illustrating blebbing as a morphodynamic signaling hub.

FIG. 10A-10C show data and analysis of SEPT6-GFP localization and the effects of septin inhibition. FIG. 10A shows mouse SEPT6-GFP probe localization in MV3 cells embedded in soft bovine collagen. Maximum intensity projections (left) and single z-slices of 0.16 micron thickness (right) shown for representative cells in two columns. FIG. 10B shows maximum intensity projections of mouse SEPT6-GFP probe localization in MV3 cells with and without inhibition by FCF or SEPT2 (33-306) expression. Cells embedded in soft bovine collagen. (Below) Fraction of cell surface comprised of blebs for MV3 cells with and without SEPT6-GFP expression, FCF treatment, and SEPT2 (33-306) expression. Dashed lines separate quartiles. Dots represent individual cells. FIG. 10C shows mouse SEPT6-GFP probe localization in MV3 cells embedded in soft bovine collagen. Maximum intensity projections and single z-slices of 0.16 micron thickness shown for representative cells in two columns. (Above) Cells expressing the SEPT2 (33-306) mutant. (Below) Cells expressing cytosolic mCherry (not shown) using the same pLVX-IRES-Hyg vector as the SEPT2 (33-306) construct. Included as a negative control demonstrating that the septin mislocalization in SEPT2 (33-306)-expressing cells is not due to effects arising from the protein expression construct.

FIG. 11A-11E shows data and images depicting endogenous septin localization via immunofluorescence. FIG. 11A shows MV3 cells adhered to fibronectin-coated glass slides showing either the mouse SEPT6-GFP probe or anti-SEPT2 immunofluorescence localization. While the septin localization to actin stress fibers (is apparent in both samples, the anti-SEPT2 signal is irregular, punctate, and possesses low signal-to-noise ratio (SNR) compared to SEPT6-GFP. Scale bars are 10 μm. FIG. 11B shows anti-SEPT2 signal in a rounded blebbing MV3 cell embedded in soft bovine collagen, with maximum intensity projections (top) and single z-slices of 0.16 micron thickness (below). Phalloidin signal from the same cell shown on right to visualize the cell surface. Anti-SEPT2 signal possesses low SNR, as seen in adhered cells. Despite this, the signal is enriched at the cell surface in blebby regions of the cell just as seen in cells expressing the SEPT6-GFP probe (see FIG. 10A). FIG. 11C shows cell surface distribution of anti-SEPT2 signal for the cell shown in FIG. 11B. FIG. 11D shows local anti-SEPT2 or SEPT6-GFP intensity and intracellular mean curvature as a function of distance from bleb edges. Mean intensity is comprised only of the brightest 50% of the cell surface to account for punctate and discontinuous immunofluorescent signal. FIG. 11E shows fraction of cortical voxels (within 0.96 μm of surface) with anti-SEPT2 intensity higher than cytoplasmic mean intensity in MV3 cells with and without expression of SEPT6-GFP. Dashed lines separate quartiles and dots represent individual cells.

FIGS. 12A-12C provides a graphical illustration and data relating to a SEPT2 (33-306) mutant construct. FIG. 12A provides a schematic illustrating the SEPT2 (33-306) mutant construct along with representative micrographs showing maximum intensity z-projections of SEPT6-GFP carrying MV3 cells, without and with expression of the SEPT2 (33-306) mutant. FIG. 12B depicts a difference in mean local SEPT6-GFP intensity compared to local maxima as a function of distance from bleb edge for cells expressing SEPT2 (33-306) and control cells. FIG. 12C is a western blot of SEPT2 in MV3 cells with and without SEPT2 (33-306) expression. Calculated mass of SEPT2 is 41.5 kD and SEPT2 (33-306) is 31.7 kD. Annotations show positions of the indicated ladder bands.

FIG. 13A-13C depicts analysis of high-speed timelapse SEPT6-GFP data in MV3. FIG. 13A shows autocorrelation curves for the local mean curvature and SEPT6-GFP data presented in FIG. 2O; the fluorescence signal was normalized to account for photobleaching as described in Methods. FIG. 13B shows temporal cross-correlation functions as shown in FIG. 2O, here with extended range of time lags. FIG. 13C shows mean rates of absolute change in SEPT6-GFP intensity for positive and negative intracellular mean curvature contigs, expressed as a function of the dynamicity of the contigs they occur within. Dynamicity is defined as the total number of timepoints showing intensity increases and decreases that occur within a contig, divided by the total number of timepoints within that contig. Derived from the same dataset used in FIG. 2O. Vertical dashed line represents the threshold for low and high dynamic groups as shown in FIG. 2O.

FIG. 14 shows changes in septin expression upon MAPKi treatment of A375 cells. Top, Western blots of SEPT2, SEPT6, SEPT7, and SEPT9 abundance (the 4 septins with highest expression in A375 cells, as measured by mass spectrometry) upon combined treatment with Dabrafenib and Trametinib at doses indicated. Three independent experiments were performed, with data from all replicates shown alongside vinculin loading controls. Bottom, densitometry analysis of Western blots showing fold change in septin expression as dose response curves. Dashed lines indicate individual experiments (color-coded according to the color bars indicating Western blot replicates); solid red lines indicate means of all replicates.

FIG. 15 shows cell viability of adhered and detached melanoma cells expressing SEPT2 (33-306). Cells grown for 24 h and assayed for cell death using ethidium homodimer staining. All treatment groups grown and assayed in simultaneous paired experiments. Dots represent individual experiments. Cell counts for all replicates: MV3 (951, 872), A375 (653, 1302). MV3 and A375 datasets were tested using one-sided Paired T Tests, p=1.02×10⁻³ and p=0.388.

FIG. 16A-16C shows anoikis resistance in non-malignant cells. FIG. 16A shows SEPT6-GFP localization in a recently detached HEK cell, embedded in soft bovine collagen. Top, maximum intensity projection (MIP); bottom, individual 0.16 μm z-slice from the same cell. FIG. 16B shows an additional fraction of MV3 cells showing caspase activation after 4 h of treatment with 10 μg/mL WGA (compared to matched, same-day negative controls) for adhered and detached cells. Total cell counts with control in parenthesis: Replicate 1: Adh 143 (220), Det 310 (544); Replicate 2: Adh 182 (152), Det 256 (215); Replicate 3: Adh 134 (157), Det 89 (267). FIG. 16C shows the same data shown in FIG. 5C, without normalization. Solid, dashed, and dotted lines represent paired, same-day experiments. Total cell counts for each condition, with adhered group in parenthesis: Replicate 1: Con 49 (89), DYN2 (K44A) 38 (55), DYN2 (K44A)+WGA 32 (25), DYN2 (K44A)+FCF 27 (33); Replicate 2: Con 23 (25), DYN2 (K44A) 28 (39), DYN2 (K44A)+WGA 27 (43), DYN2 (K44A)+FCF 28 (34); Replicate 3: Con 92 (54), DYN2 (K44A) 121 (84), DYN2 (K44A)+WGA 101 (115), DYN2 (K44A)+FCF 141 (70).

FIG. 17 shows paired mean differences between control and experimental condition shown in Gardner-Altman estimation plots for the data presented in FIGS. 1H-11, 3B-3F, 4F, 4G, and 4H, as indicated. Gray distributions represent 5000 bootstrapped samples, black bars represent 95% confidence intervals, black dots represent mean difference.

FIGS. 18A-18B depict images of MV3 melanoma cells grown in vitro and in vivo (FIG. 18A) as well as high magnification images of septin structure in the invasive front but not tumor core of xenograft cells (FIG. 18B).

FIG. 19 depicts bleb-associated septin structures in KRAS mutant-driven SW480 colon cancer line and EGFR-driven HCC827 lung adenocarcinoma cancer line.

FIGS. 20A-20B shows plots depicting dimensions (FIG. 20A) and intensity (FIG. 20B) of detected septin structures in melanoma xenografts in mice treated with vehicle (control) or forchlorfeneuron (FCF).

FIG. 21 shows illustrative images of bleb associated septin-signaling hubs in cells found in a biopsy of a patient with mucosal melanoma.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present disclosure. The drawings and description are intended to describe aspects and embodiments of the present disclosure in sufficient detail to enable those skilled in the art to practice the present disclosure. Other components can be utilized and changes can be made without departing from the scope of the present disclosure. The following description is, therefore, not to be taken in a limiting sense.

The present disclosure is based, at least in part, on identification of a novel morphology-driven survival signaling pathway. In this innovative paradigm of survival signaling, information flow is transduced from chemical (pro-amoeboid morphogenic signaling), to spatial (blebbiness and plasma membrane curvature), and back to chemical (survival signaling), with septins representing a crucial bottleneck as the sole spatiochemical translator of this signal. Bleb formation has long been recognized as a morphological program associated with melanoma cells and metastasis. While this morphological program has been primarily interpreted as a means of amoeboid migration, the present disclosure demonstrates that dynamic blebbing prompts the construction of septin signaling hubs that substitute for the loss of anchorage-dependent signaling activity upon substrate detachment. Once constructed, such signaling can provide improved survival for cancer cells traveling through low-adhesion environments such as blood and lymph. Because septins serve as the single node bridging blebbing with downstream survival signaling, targeting this lynchpin of bleb signaling may be a therapeutic strategy, particularly for cancer and prevention of cancer cell metastasis.

Provided herein are compositions and methods for treating cancer and, in some aspects, a malignant solid tumor. In certain embodiments, a septin inhibitor and/or one or more additional therapies (e.g., cancer therapies (e.g., targeted MAPKi inhibitors, targeted BRAF inhibitors) can be provided to inhibit cancer cell motility, inhibit metastasis, and/or treat cancer.

### I. Terminology

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or any combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, “prevent” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

The term “effective amount” or “therapeutically effective amount” refers to an amount sufficient to effect beneficial or desirable biological and/or clinical results.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject can be a human. In other embodiments, the subject can be a human in need of treating a cancer (e.g., a melanoma).

### II. Compositions

In certain embodiments, compositions disclosed herein may comprise at least one septin inhibitor. In certain embodiments, compositions disclosed herein may comprise at least one bleb inhibitor. In some embodiments, compositions disclosed herein may comprise a combination of at least one septin inhibitor and at least one targeted MAPK signaling pathway inhibitor (MAPKi) agent and/or at least one or more targeted BRAF inhibitor agents. In some embodiments, compositions disclosed herein may comprise a combination of at least one bleb inhibitor and at least one targeted MAPK signaling pathway inhibitor (MAPKi) agent and/or at least one or more targeted BRAF inhibitor agents. In some embodiments, compositions disclosed herein may be pharmaceutical compositions.

### (a) Septins

Septins are a family of proteins that play important roles in many cellular processes by providing rigidity to the cell membrane, serving as scaffolds to recruit proteins to specific subcellular locales, and/or creating membrane diffusion barriers to establish discrete cellular domains. Mammalian septins comprise 13 family members and all known septins are composed of a highly conserved central GTP-binding region flanked by N- and C-terminal with variable length. In some embodiments, septin family members refer to any of septin-1, septin-2, septin-3, septin-5, septin-6, septin-7, septin-8, septin-9, septin-10, septin-11, septin-12, or septin-14.

In certain embodiments, compositions disclosed herein may include at least one septin inhibitor. As used herein, a “septin inhibitor” can include any biomolecule(s) that can inhibit septin direct activity, inhibit septin indirect activity, inhibit formation of a septin-bleb complex, decrease expression of a septin gene, decrease expression of a septin protein, or any combination thereof. In some embodiments, compositions having a septin inhibitor can include any biomolecule(s) that are modulators and/or inhibitors of targets upstream or downstream of a septin signaling cascade that would effectively inhibit the physiological outcome of septin inhibition. In some embodiments, biomolecule(s) capable of inhibiting one or more septins can be a peptide, an antibody, a chemical, a compound, an oligo, a nucleic acid molecule, or any combination thereof.

In certain embodiments, a septin inhibitor for use in compositions herein can be a pan septin inhibitor. As used herein, “a pan septin inhibitor” refers to an inhibitor that inhibits all septin family members. In some embodiments, a septin inhibitor for use in compositions herein can inhibit one or more—but not all—septin family members. In some embodiments, a septin inhibitor for use in compositions herein can inhibit the activity of one septin family member. In accordance with these embodiments, compositions disclosed herein may include a septin-1 inhibitor, a septin-2 inhibitor, a septin-3 inhibitor, a septin-4 inhibitor, a septin-5 inhibitor, a septin-6 inhibitor, a septin-7 inhibitor, a septin-8 inhibitor, a septin-9 inhibitor, a septin-10 inhibitor, a septin-11 inhibitor, a septin-12 inhibitor, a septin-14 inhibitor or any combination thereof.

In certain embodiments, septin inhibitors for use in compositions herein can include a nucleic acid molecule. The term “nucleic acid molecule” as used herein refers to a molecule having nucleotides. The nucleic acid can be single, double, or multiple stranded and may comprise modified or unmodified nucleotides or non-nucleotides or various mixtures and combinations thereof. In some aspects, a nucleic acid molecule for use herein can be a double-stranded RNA. In some other aspects a double stranded RNA suitable for use herein can be small temporal RNA, small nuclear RNA, small nucleolar RNA, short hairpin RNA, microRNA, or the like.

In certain embodiments, septin inhibitors for use in compositions herein may be a polypeptide, a protein, a peptide, a protein fragment, an antibody, or any combination thereof. As used herein, the terms “peptide,” “polypeptide,” and “protein,” refer to polymers comprised of amino acid monomers linked by amide bonds. Peptides may include the standard 20 α-amino acids that are used in protein synthesis by cells (i.e., natural amino acids), as well as non-natural amino acids (non-natural amino acids may be found in nature, but not used in protein synthesis by cells, e.g., ornithine, citrulline, and sarcosine, or may be chemically synthesized), amino acid analogs, and peptidomimetics. In some embodiments, a peptide inhibitor of septin may be fused at its C-terminus, its N-terminus, or both with at least one other peptide and/or polypeptide. In some aspects, the at least one other peptide or polypeptide may be a carrier peptide, allowing cell penetration of the resulting fusion peptide. As an example, a peptide inhibitor of septin may be fused to a cell penetrating peptide (CPP). CPPs are short peptides that facilitate cellular uptake of various molecular cargo, e.g. via endocytosis. Non-limiting examples of CPP that may be suitable for use herein include Antennapedia Penetratin, HIV-1 TAT protein, pVEC Cadherin, Transportan Galanine/Mastoparan, MPG HIV-gp41/SV40 T-antigen, Pep-1 HIV-reverse transcriptase/SV40 T-antigen, Polyarginines, MAP, R6W3, NLS, 8-lysines, ARF (1-22), Azurin-p28, and the like. As another example, a peptide inhibitor of septin may be fused to cell-targeting peptides (CTPs). CTPs are ideal carrier molecules as that bind with high affinity to overexpressed receptors on the tumor cell surface, effectively targeting a peptide inhibitor of septin to the target tumor. CTPs can target, for example integrin receptors, epidermal growth factor receptors (EGFR), neuropeptide Y (NPY) receptors, gastrin-releasing peptide receptors (GRPR), somatostatin receptors (e.g., SSTR2), gonadotropin-releasing hormone receptors (GnRHR), vasoactive intestinal peptide (VIP) receptors, melanocortin 1 receptors (MC1R), neurotensin receptors (e.g., NTSR1), and the like.

In certain embodiments septin inhibitors for use in compositions herein may be a septin antibody. As used herein, the terms “septin antibody” or “antibody” can refer to an antibody capable of binding to and/or blocking expression of or activities of a protein encoded by a septin gene. In some embodiments, antibodies acting as an inhibitor of septin may be full-length antibodies, antigen binding fragments of full-length antibodies, Fab fragments, single chain antibodies (scFv), diabodies, triabodies, minibodies, nanobodies, single-domain antibodies, camelids, or any combination thereof. In some embodiments, septin antibodies disclosed herein may be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the antibodies described herein, e.g., septin antibodies, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In certain embodiments septin inhibitors for use in compositions herein may be a compound (also referred to herein as “small molecule”). In some embodiments, a septin inhibitor compound may be forchlorfenuron (FCF). In some embodiments, a septin inhibitor compound may be a FCF analog. In accordance with these embodiments, an analog of FCF suitable for use herein may be UR214-1, UR214-7, UR214-9, or any combination thereof.

In certain embodiments, inhibitors of septin(s) disclosed herein can be used to treat, attenuate, or prevent tumor growth and/or progression. In certain embodiments, inhibitors of septin(s) disclosed herein can be used to treat, attenuate, or prevent tumor metastasis. In certain embodiments inhibitors of septin(s) disclosed herein can be used to treat, attenuate, or prevent treatment-resistant tumor growth and/or metastasis. In certain embodiments, inhibitors of septin(s) disclosed herein can be used to increase the therapeutic effect of one or more cancer treatments.

### (b) Blebs

Acquisition of anoikis resistance is a pivotal step in cancer disease progression, as metastasizing cancer cells often lose firm attachment to surrounding tissue. In these poorly attached states, cells often adopt rounded morphologies and form small hemispherical plasma membrane protrusions called blebs. Blebs are spherical plasma membrane protrusions formed when the membrane detaches from the underlying cortex. As provided in the exemplary methods herein, blebbing generates plasma membrane contours that recruit curvature sensing septin proteins and triggers the formation of membrane-proximal signaling hubs that initiate signaling cascades leading to anoikis resistance.

In certain embodiments, compositions disclosed herein may include at least one bleb inhibitor. As used herein, a “bleb inhibitor” can include any biomolecule(s) that can inhibit blebbing activity directly, inhibit blebbing activity indirectly, inhibit formation of a septin-bleb complex, or any combination thereof. In some embodiments, compositions having a bleb inhibitor can include any biomolecule(s) that are modulators and/or inhibitors of targets upstream or downstream of a bleb signaling cascade that would effectively inhibit the physiological outcome of bleb inhibition. In some embodiments, biomolecule(s) capable of inhibiting blebbing can be a peptide, an antibody, a chemical, a compound, an oligo, a nucleic acid molecule, or any combination thereof.

In certain embodiments, bleb inhibitors for use in compositions herein can include a nucleic acid molecule. The term “nucleic acid molecule” as used herein refers to a molecule having nucleotides. The nucleic acid can be single, double, or multiple stranded and may comprise modified or unmodified nucleotides or non-nucleotides or various mixtures and combinations thereof. In some aspects, a nucleic acid molecule for use herein can be a double-stranded RNA. In some other aspects a double stranded RNA suitable for use herein can be small temporal RNA, small nuclear RNA, small nucleolar RNA, short hairpin RNA, microRNA, or the like.

In certain embodiments, bleb inhibitors for use in compositions herein may be a polypeptide, a protein, a peptide, a protein fragment, an antibody, or any combination thereof. As used herein, the terms “peptide,” “polypeptide,” and “protein,” refer to polymers comprised of amino acid monomers linked by amide bonds.

In certain embodiments bleb inhibitors for use in compositions herein may be a bleb antibody. As used herein, the terms “bleb antibody” or “antibody” can be involved in blebbing. In some embodiments, antibodies acting as an inhibitor of blebbing may be full-length antibodies, antigen binding fragments of full-length antibodies, Fab fragments, single chain antibodies (scFv), diabodies, triabodies, minibodies, nanobodies, single-domain antibodies, camelids, or any combination thereof. In some embodiments, bleb antibodies disclosed herein may be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the antibodies described herein, e.g., bleb antibodies, can be either monoclonal or polyclonal.

In certain embodiments bleb inhibitors for use in compositions herein may be a compound. In some embodiments, a bleb inhibitor compound may be a lectin. In some aspects, a lectin for use as a bleb inhibitor herein may be a glycan binding lectin. Non-limiting examples of glycan binding lectins include Ricinus communis agglutinin-1 (RCA-1), wheat germ agglutinin (WVGA), Erythrina cristagalli (ECL), Peanut agglutinin (PNA), Maackia amurensis lectin-1 (MAL-1), Sambucus nigra (SNA), and the like. In some embodiments, bleb inhibitors for use in compositions herein may be wheat germ agglutinin.

In certain embodiments, inhibitors of blebbing disclosed herein can be used to treat, attenuate, or prevent tumor growth and/or progression. In certain embodiments, inhibitors of blebbing disclosed herein can be used to treat, attenuate, or prevent tumor metastasis. In certain embodiments inhibitors of blebbing disclosed herein can be used to treat, attenuate, or prevent treatment-resistant tumor growth and/or metastasis. In certain embodiments, inhibitors of blebbing disclosed herein can be used to increase the therapeutic effect of one or more cancer treatments.

### (c) Targeted Therapies

Targeted therapies are drugs or other substances that block the growth and spread of cancer by interfering with specific molecules (“molecular targets”) that are involved in the growth, progression, and/or spread of cancer. Targeted cancer therapies are sometimes called “molecularly targeted drugs,” “molecularly targeted therapies,” “precision medicines,” or similar names. Non-limiting examples of targeted therapies include hormone therapies, signal transduction inhibitors, gene expression modulators, apoptosis inducers, angiogenesis inhibitors, immunotherapies, toxin delivery molecules, and the like.

In certain embodiments, compositions disclosed herein may comprise a combination of 1) at least one septin inhibitor and/or at least one inhibitor of blebbing; and ii) and at least one targeted therapy. In some aspects, targeted therapies disclosed herein may be a small molecule, an antibody, or the like. One of skill in the art will appreciate that the appropriate targeted therapies for use herein will depend on, for example, the type of cancer, the stage of cancer, if the cancer is associated with a gene mutation, and the like. The following targeted therapies associated with one or more cancer types are non-limiting examples of suitable targeted therapies for use in the compositions and methods of the present disclosure: 1) Bladder cancer: Atezolizumab (Tecentriq), nivolumab (Opdivo), avelumab (Bavencio), pembrolizumab (Keytruda), erdafitinib (Balversa), enfortumab vedotin-ejfv (Padcev), sacituzumab govitecan-hziy (Trodelvy); 2) Brain cancer: Bevacizumab (Avastin), everolimus (Afinitor), belzutifan (Welireg); 3) Breast cancer: Everolimus (Afinitor), tamoxifen (Nolvadex), toremifene (Fareston), trastuzumab (Herceptin), fulvestrant (Faslodex), anastrozole (Arimidex), exemestane (Aromasin), lapatinib (Tykerb), letrozole (Femara), pertuzumab (Perjeta), ado-trastuzumab emtansine (Kadcyla), palbociclib (Ibrance), ribociclib (Kisqali), neratinib maleate (Nerlynx), abemaciclib (Verzenio), olaparib (Lynparza), talazoparib tosylate (Talzenna), alpelisib (Piqray), fam-trastuzumab deruxtecan-nxki (Enhertu), tucatinib (Tukysa), sacituzumab govitecan-hziy (Trodelvy), pertuzumab, trastuzumab, and hyaluronidase-zzxf (Phesgo), pembrolizumab (Keytruda), margetuximab-cmkb (Margenza); 4) Cervical cancer: Bevacizumab (Avastin), pembrolizumab (Keytruda), tisotumab vedotin-tftv (Tivdak); 5) Colorectal cancer: Cetuximab (Erbitux), panitumumab (Vectibix), bevacizumab (Avastin), ziv-aflibercept (Zaltrap), regorafenib (Stivarga), ramucirumab (Cyramza), nivolumab (Opdivo), ipilimumab (Yervoy), encorafenib (Braftovi), pembrolizumab (Keytruda); 6) Dermatofibrosarcoma protuberans: Imatinib mesylate (Gleevec); 7) Endocrine/neuroendocrine tumors: Lanreotide acetate (Somatuline Depot), avelumab (Bavencio), lutetium Lu 177-dotatate (Lutathera), iobenguane | 131 (Azedra); 8) Endometrial cancer: Pembrolizumab (Keytruda), lenvatinib mesylate (Lenvima), dostarlimab-gxly (Jemperli); 9) Esophageal cancer: Trastuzumab (Herceptin), ramucirumab (Cyramza), pembrolizumab (Keytruda), nivolumab (Opdivo), fam-trastuzumab deruxtecan-nxki (Enhertu); 10) Head and neck cancer: Cetuximab (Erbitux), pembrolizumab (Keytruda), nivolumab (Opdivo); 11) Gastrointestinal stromal tumor: Imatinib mesylate (Gleevec), sunitinib (Sutent), regorafenib (Stivarga), avapritinib (Ayvakit), ripretinib (Qinlock); 12) Giant cell tumor: Denosumab (Xgeva), pexidartinib hydrochloride (Turalio); 13) Kidney cancer: Bevacizumab (Avastin), sorafenib (Nexavar), sunitinib (Sutent), pazopanib (Votrient), temsirolimus (Torisel), everolimus (Afinitor), axitinib (Inlyta), nivolumab (Opdivo), cabozantinib (Cabometyx), lenvatinib mesylate (Lenvima), ipilimumab (Yervoy), pembrolizumab (Keytruda), avelumab (Bavencio), tivozanib hydrochloride (Fotivda), belzutifan (Welireg); 14) Leukemia: Tretinoin (Vesanoid), imatinib mesylate (Gleevec), dasatinib (Sprycel), nilotinib (Tasigna), bosutinib (Bosulif), rituximab (Rituxan), alemtuzumab (Campath), ofatumumab (Arzerra), obinutuzumab (Gazyva), ibrutinib (Imbruvica), idelalisib (Zydelig), blinatumomab (Blincyto), venetoclax (Venclexta), ponatinib hydrochloride (Iclusig), midostaurin (Rydapt), enasidenib mesylate (Idhifa), inotuzumab ozogamicin (Besponsa), tisagenlecleucel (Kymriah), gemtuzumab ozogamicin (Mylotarg), rituximab and hyaluronidase human (Rituxan Hycela), ivosidenib (Tibsovo), duvelisib (Copiktra), moxetumomab pasudotox-tdfk (Lumoxiti), glasdegib maleate (Daurismo), gilteritinib (Xospata), tagraxofusp-erzs (Elzonris), acalabrutinib (Calquence), avapritinib (Ayvakit), brexucabtagene autoleucel (Tecartus), asciminib hydrochloride (Scemblix); 15) Liver and bile duct cancer: Sorafenib (Nexavar), regorafenib (Stivarga), nivolumab (Opdivo), lenvatinib mesylate (Lenvima), pembrolizumab (Keytruda), cabozantinib (Cabometyx), ramucirumab (Cyramza), ipilimumab (Yervoy), pemigatinib (Pemazyre), atezolizumab (Tecentriq), bevacizumab (Avastin), infigratinib phosphate (Truseltiq), ivosidenib (Tibsovo); 16) Lung cancer: Bevacizumab (Avastin), crizotinib (Xalkori), erlotinib (Tarceva), gefitinib (Iressa), afatinib dimaleate (Gilotrif), ceritinib (LDK378/Zykadia), ramucirumab (Cyramza), nivolumab (Opdivo), pembrolizumab (Keytruda), osimertinib (Tagrisso), necitumumab (Portrazza), alectinib (Alecensa), atezolizumab (Tecentriq), brigatinib (Alunbrig), trametinib (Mekinist), dabrafenib (Tafinlar), durvalumab (Imfinzi), dacomitinib (Vizimpro), lorlatinib (Lorbrena), entrectinib (Rozlytrek), capmatinib hydrochloride (Tabrecta), ipilimumab (Yervoy), selpercatinib (Retevmo), pralsetinib (Gavreto), cemiplimab-rwlc (Libtayo), tepotinib hydrochloride (Tepmetko), sotorasib (Lumakras), amivantamab-vmjw (Rybrevant), mobocertinib succinate (Exkivity); 17) Lymphoma: Ibritumomab tiuxetan (Zevalin), denileukin diftitox (Ontak), brentuximab vedotin (Adcetris), rituximab (Rituxan), vorinostat (Zolinza), romidepsin (Istodax), bexarotene (Targretin), bortezomib (Velcade), pralatrexate (Folotyn), ibrutinib (Imbruvica), siltuximab (Sylvant), belinostat (Beleodaq), obinutuzumab (Gazyva), nivolumab (Opdivo), pembrolizumab (Keytruda), rituximab and hyaluronidase human (Rituxan Hycela), copanlisib hydrochloride (Aliqopa), axicabtagene ciloleucel (Yescarta), acalabrutinib (Calquence), tisagenlecleucel (Kymriah), venetoclax (Venclexta), mogamulizumab-kpkc (Poteligeo), duvelisib (Copiktra), polatuzumab vedotin-piiq (Polivy), zanubrutinib (Brukinsa), tazemetostat hydrobromide (Tazverik), selinexor (Xpovio), tafasitamab-cxix (Monjuvi), brexucabtagene autoleucel (Tecartus), crizotinib (Xalkori), umbralisib tosylate (Ukoniq), lisocabtagene maraleucel (Breyanzi), loncastuximab tesirine-lpyl (Zynlonta); 18) Malignant mesothelioma: Ipilimumab (Yervoy), nivolumab (Opdivo); 19) Microsatellite instability-high or mismatch repair-deficient solid tumors: Pembrolizumab (Keytruda), dostarlimab-gxly (Jemperli); 20) Multiple myeloma: Bortezomib (Velcade), carfilzomib (Kyprolis), daratumumab (Darzalex), ixazomib citrate (Ninlaro), elotuzumab (Empliciti), selinexor (Xpovio), isatuximab-irfc (Sarclisa), daratumumab and hyaluronidase-fihj (Darzalex Faspro), belantamab mafodotin-blmf (Blenrep), idecabtagene vicleucel (Abecma), ciltacabtagene autoleucel (Carvykti); 21) Myelodysplastic/myeloproliferative disorders: Imatinib mesylate (Gleevec), ruxolitinib phosphate (Jakafi), fedratinib hydrochloride (Inrebic), pacritinib citrate (Vonjo); 22) Neuroblastoma: Dinutuximab (Unituxin), naxitamab-gqgk (Danyelza); 23) Ovarian epithelial/fallopian tube/primary peritoneal cancers: Bevacizumab (Avastin), olaparib (Lynparza), rucaparib camsylate (Rubraca), niraparib tosylate monohydrate (Zejula); 24) Pancreatic cancer: Erlotinib (Tarceva), everolimus (Afinitor), sunitinib (Sutent), olaparib (Lynparza), belzutifan (Welireg); 25) Plexiform neurofibroma: Selumetinib sulfate (Koselugo); 26) Prostate cancer: Cabazitaxel (Jevtana), enzalutamide (Xtandi), abiraterone acetate (Zytiga), radium 223 dichloride (Xofigo), apalutamide (Erleada), darolutamide (Nubeqa), rucaparib camsylate (Rubraca), olaparib (Lynparza); 27) Skin cancer: Vismodegib (Erivedge), sonidegib (Odomzo), ipilimumab (Yervoy), vemurafenib (Zelboraf), trametinib (Mekinist), dabrafenib (Tafinlar), pembrolizumab (Keytruda), nivolumab (Opdivo), cobimetinib (Cotellic), alitretinoin (Panretin), avelumab (Bavencio), encorafenib (Braftovi), binimetinib (Mektovi), cemiplimab-rwlc (Libtayo), atezolizumab (Tecentriq), tebentafusp-tebn (Kimmtrak), nivolumab and relatlimab-rmbw (Opdualag); 28) Soft tissue sarcoma: Pazopanib (Votrient), alitretinoin (Panretin), tazemetostat hydrobromide (Tazverik), sirolimus protein-bound particles (Fyarro); 29) Solid tumors that are tumor mutational burden-high (TMB-H): Pembrolizumab (Keytruda); 30) Solid tumors with an NTRK gene fusion: Larotrectinib sulfate (Vitrakvi), entrectinib (Rozlytrek); 31) Stomach (gastric) cancer: Pembrolizumab (Keytruda), trastuzumab (Herceptin), ramucirumab (Cyramza), fam-trastuzumab deruxtecan-nxki (Enhertu), nivolumab (Opdivo); 32) Systemic mastocytosis: Imatinib mesylate (Gleevec), midostaurin (Rydapt), avapritinib (Ayvakit); and 33) Thyroid cancer: Cabozantinib (Cometriq), vandetanib (Caprelsa), sorafenib (Nexavar), lenvatinib mesylate (Lenvima), trametinib (Mekinist), dabrafenib (Tafinlar), selpercatinib (Retevmo), pralsetinib (Gavreto).

In certain embodiments, a targeted therapy for use in the compositions disclosed herein may comprise one or more signal transduction inhibitors. Signal transduction inhibitors block the activities of molecules that participate in signal transduction, the process by which a cell responds to signals from its environment. During this process, once a cell has received a specific signal, the signal is relayed within the cell through a series of biochemical reactions that ultimately produce the appropriate response(s). In some cancers, the malignant cells are stimulated to divide continuously without being prompted to do so by external growth factors. Signal transduction inhibitors interfere with this inappropriate signaling. In some embodiments, signal transduction inhibitors for use herein may block the activities of molecules associated with one or more morphology-driven survival signaling pathways. In some embodiments, signal transduction inhibitors for use herein may inhibit one or more MAPK signaling pathways (i.e., targeted MAPK signaling pathway inhibitor (MAPKi) agents). In some embodiments, one or more targeted MAPKi agents for use in the compositions and methods disclosed herein may comprise PD-325901, TAK-733, binimetinib, cobimetinib, selumetinib, trametinib, gefitinib, lapatinib, ARRY-614, ralimetinib, ulixertinib, erlotinib, bevacizumab, or any combination thereof.

In some embodiments, signal transduction inhibitors for use herein may selectively target BRAF kinase (i.e., targeted BRAF inhibitor agents, or BRAFi) and thus interfere with the MAPK signaling pathway that regulates the proliferation and survival of cancer cells. In some embodiments, one or more targeted BRAFi agents for use in the compositions and methods disclosed herein may comprise vemurafenib, dabrafenib, encorafenib, PLX8394, CCT3833, LY3009120, lifirafenib, belvarafinib, TAK-580, RO5126766, trametiglue or any combination thereof.

In some embodiments, signal transduction inhibitors for use herein may selectively target RAS proteins. RAS proteins, their regulators and the downstream enzymes that they control are activated in many tumor types by a variety of mechanisms, including oncogenic mutation of RAS genes. Indeed, mutations in RAS are the most common oncogenic driver mutations in human cancer, present in approximately 30% of all cancers. In some embodiments, a RAS signal transduction inhibitor may inhibit RAS directly or indirectly. Non-limiting examples of indirect RAS inhibitors include II-B08, 11α-1, PHPS1, GS493, SHP099, TNO155, JAB-3068, RMC-4630, Tipifarnib (R115777), Lonafarnib (SCH-66336), Deltarasin, Deltazinone 1, Deltasonamide 1 and 2, Bisphosphates/zoledronic acid, and the like. Non-limiting examples of direct RAS inhibitors include Sulindac, Cyclorasin 9A5, MCP1 and derivatives, DCAI, Kobe0065 and Kobe 2602, HBS3, SAH-SOS1A, ARS-1620, MRTX849, AMG 510, BI-2852, Y13-259, and the like.

In certain embodiments, septin inhibition according to the methods disclosed herein may act as a targeted therapeutic to MAPK signal transduction. In certain embodiments, septin inhibition according to the methods disclosed herein may act as a targeted therapeutic to BRAF signal transduction. In certain embodiments, septin inhibition according to the methods disclosed herein may act as a targeted therapeutic to RAS signal transduction.

In certain embodiments, the active agents (i.e., i) at least one septin inhibitor and/or at least one inhibitor of blebbing; and ii) and at least one targeted therapy) disclosed herein can act additively or synergistically. In some aspects, a first agent (e.g., septin inhibitor and/or bleb inhibitor) may be administered concurrently with one or more second active agents (e.g., targeted therapy) in the same composition. In some other aspects, a first agent (e.g., septin inhibitor and/or bleb inhibitor) may be administered concurrently with one or more second active agents (e.g., targeted therapy) in separate compositions. In still some other aspects, a first agent (e.g., septin inhibitor and/or bleb inhibitor) may be administered prior to or subsequent to administration of a second active agent (e.g., targeted therapy). In some embodiments, compositions (e.g., comprising septin inhibitor and/or bleb inhibitor) may be effective at inhibiting or reversing resistance to certain agents, particularly targeted therapies.

### (d) Anticancer Therapies

The present disclosure can provide for use of one or more anticancer therapies in combination with any of the compositions disclosed herein. In accordance with some embodiments of the invention, the therapies that may be prescribed to a subject with increased likelihood of cancer metastases may be selected, used and/or administered to treat a cancer, a solid tumor, a metastasis, or any combination thereof. In some embodiments, one or more anticancer therapies may be any one or more of surgery, radiation, chemotherapy, immunotherapy, vaccine or combinations thereof.

In some embodiments, one or more anticancer therapies may be chemotherapy. Chemotherapeutic agents may be selected from any one or more of cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: treosulfan, and trofosfamide; plant alkaloids: vinblastine, paclitaxel, docetaxol; DNA topoisomerase inhibitors: doxorubicin, epirubicin, etoposide, camptothecin, topotecan, irinotecan, teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15, INO-1001, PJ34, 3-aminobenzamide, 4-amino-1,8-naphthalimide, 6 (5H)-phenanthridinone, benzamide, NU1025).

In some embodiments, one or more anticancer therapies may be radiation therapy. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. In some aspects, the radiation therapy can be administered as external beam radiation or teletherapy wherein the radiation is directed from a remote source. In other aspects, the radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In some embodiments, one or more anticancer therapies may be immunotherapy. Immunotherapy may comprise, for example, use of cancer vaccines and/or sensitized antigen presenting cells. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines.

In some embodiments, one or more anticancer therapies may be hormonal therapy, Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In certain embodiments, the duration and/or dose of treatment with anticancer therapies may vary according to the particular anti-cancer agent or combination thereof. An appropriate treatment time for a particular cancer therapeutic agent will be appreciated by the skilled artisan. In some embodiments, the continued assessment of optimal treatment schedules for each cancer therapeutic agent is contemplated, where the genetic signature of the cancer of the subject as determined by the methods of the invention is a factor in determining optimal treatment doses and schedules.

### (e) Pharmaceutical Formulations and Treatment Regimens

In certain embodiments, any one or more active agents disclosed herein (i.e., septin inhibitors, bleb inhibitors, targeted therapies, or any combination thereof) may be provided per se or as part of a pharmaceutical composition, where the active agent(s) can be mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

### (i) Pharmaceutically Acceptable Carriers and Excipients

Hereinafter, the phrases “physiologically acceptable carrier” and “pharmaceutically acceptable carrier” are interchangeably used herein to refer to a carrier or a diluent that does not cause significant irritation to an organism and does not abrogate the biological activity and properties of the administered compound. An adjuvant is included under these phrases.

In certain embodiments, compositions disclosed herein may further compromise one or more pharmaceutically acceptable diluent(s), excipient(s), and/or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art.

In some embodiments, pharmaceutical compositions herein may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, or any combination thereof. Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In certain embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In some embodiments, any of the well-known techniques, carriers, and excipients may be used as suitable and/or as understood in the art.

In certain embodiments, pharmaceutical compositions described herein may be an aqueous suspension comprising one or more polymers as suspending agents. In some embodiments, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or any combination thereof. In some embodiments, pharmaceutical compositions disclosed herein may comprise at least 5%, at least 10%, at least 20%, at least 25%, at least 30%, at least 35%, at least 40%, at least 45%, or at least 50% total amount of polymers as suspending agent(s) by total weight of the composition. In some embodiments, pharmaceutical compositions disclosed herein may comprise about 5% to about 99%, about 10%, about 95%, or about 15% to about 90% total amount of polymers as suspending agent(s) by total weight of the composition.

In certain embodiments, pharmaceutical compositions disclosed herein may comprise a viscous formulation. In some embodiments, viscosity of composition herein may be increased by the addition of one or more gelling or thickening agents. In some embodiments, compositions disclosed herein may comprise one or more gelling or thickening agents in an amount to provide a sufficiently viscous formulation to remain on treated tissue. In some embodiments, pharmaceutical compositions disclosed herein may comprise at least 5%, at least 10%, at least 20%, at least 25%, at least 30%, at least 35%, at least 40%, at least 45%, or at least 50% total amount of gelling or thickening agent(s) by total weight of the composition. In some embodiments, pharmaceutical compositions disclosed herein may comprise about 5% to about 99%, about 10%, about 95%, or about 15% to about 90% total amount of gelling or thickening agent(s) by total weight of the composition. In some embodiments, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or any combination thereof.

In certain embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some embodiments, pharmaceutical compositions disclosed herein may comprise at least 5%, at least 10%, at least 20%, at least 25%, at least 30%, at least 35%, at least 40%, at least 45%, or at least 50% total amount of one or more agents by total weight of the composition. In some embodiments, pharmaceutical compositions disclosed herein may comprise about 5% to about 99%, about 10%, about 95%, or about 15% to about 90% total amount of one or more agents by total weight of the composition. In some embodiments, one or more of these agents may be added to improve the performance, efficacy, safety, shelf-life and/or other property of the muscarinic antagonist composition of the present disclosure. In some embodiments, additives may be biocompatible, without being harsh, abrasive, and/or allergenic.

In certain embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some embodiments, any pharmaceutically acceptable organic or inorganic acid may be used. In some embodiments, pharmaceutical compositions disclosed herein may comprise at least 5%, at least 10%, at least 20%, at least 25%, at least 30%, at least 35%, at least 40%, at least 45%, at least 50% total amount of one or more acidifying agents by total weight of the composition. In some embodiments, pharmaceutical compositions disclosed herein may comprise about 5% to about 99%, about 10%, about 95%, or about 15% to about 90% total amount of one or more acidifying agents by total weight of the composition.

In certain embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some embodiments, any pharmaceutically acceptable organic or inorganic base can be used. In some embodiments, pharmaceutical compositions disclosed herein may comprise at least 5%, at least 10%, at least 20%, at least 25%, at least 30%, at least 35%, at least 40%, at least 45%, at least 50% total amount of one or more alkalizing agents by total weight of the composition. In some embodiments, pharmaceutical compositions disclosed herein may comprise about 5% to about 99%, about 10%, about 95%, or about 15% to about 90% total amount of one or more alkalizing agents by total weight of the composition.

In certain embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate, sodium metabisulfite and other materials known to one of ordinary skill in the art. In some embodiments, pharmaceutical compositions disclosed herein may comprise at least 5%, at least 10%, at least 20%, at least 25%, at least 30%, at least 35%, at least 40%, at least 45%, at least 50% total amount of one or more antioxidants by total weight of the composition. In some embodiments, pharmaceutical compositions disclosed herein may comprise about 5% to about 99%, about 10%, about 95%, or about 15% to about 90% total amount of one or more antioxidants by total weight of the composition.

In certain embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some embodiments, any pharmaceutically acceptable organic or inorganic buffer can be used. In some embodiments, pharmaceutical compositions disclosed herein may comprise at least 5%, at least 10%, at least 20%, at least 25%, at least 30%, at least 35%, at least 40%, at least 45%, at least 50% total amount of one or more buffering agents by total weight of the composition. In some embodiments, pharmaceutical compositions disclosed herein may comprise about 5% to about 99%, about 10%, about 95%, or about 15% to about 90% total amount of one or more buffering agents by total weight of the composition.

In some embodiments, the amount of one or more buffering agents may depend on the desired pH level of a composition. In some embodiments, pharmaceutical compositions disclosed herein may have a pH of about 6 to about 9. In some embodiments, pharmaceutical compositions disclosed herein may have a pH greater than about 8, greater than about 7.5, greater than about 7, greater than about 6.5, or greater than about 6.

In certain embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some embodiments, any pharmaceutically acceptable preservative can be used. In some embodiments, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition. In some embodiments, pharmaceutical compositions disclosed herein may comprise about 5% to about 99%, about 10%, about 95%, or about 15% to about 90% total amount of one or more preservatives by total weight of the composition.

In certain embodiments, pharmaceutical compositions disclosed herein may comprise one or more surface-acting reagents or detergents. In some embodiments, surface-acting reagents or detergents may be synthetic, natural, or semi-synthetic. In some embodiments, compositions disclosed herein may comprise anionic detergents, cationic detergents, zwitterionic detergents, ampholytic detergents, amphoteric detergents, nonionic detergents having a steroid skeleton, or any combination thereof. In some embodiments, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition. In some embodiments, pharmaceutical compositions disclosed herein may comprise about 5% to about 99%, about 10%, about 95%, or about 15% to about 90% total amount of one or more surface-acting reagents or detergents by total weight of the composition.

In certain embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some embodiments, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition. In some embodiments, pharmaceutical compositions disclosed herein may comprise about 5% to about 99%, about 10%, about 95%, or about 15% to about 90% total amount of one or more stabilizers by total weight of the composition.

In some embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some embodiments, a vapor pressure depression method is used to calculate the osmolarity of the compositions disclosed herein. In some embodiments, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some embodiments, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some embodiments, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition. In some embodiments, pharmaceutical compositions disclosed herein may comprise about 5% to about 99%, about 10%, about 95%, or about 15% to about 90% total amount of one or more tonicity modifiers by total weight of the composition.

### (ii) Dosage Formulations

In certain embodiments, the present disclosure provides compositions formulated for one or more routes of administration. Suitable routes of administration may, for example, include oral, rectal, transmucosal, transnasal, intestinal, and/or parenteral delivery. In some embodiments, compositions herein formulated can be formulated for parenteral delivery. In some embodiments, compositions herein formulated can be formulated intramuscular, subcutaneous, intramedullary, intravenous, intraperitoneal, and/or intranasal injections.

In certain embodiments, one may administer a composition herein in a local or systemic manner, for example, via local injection of the pharmaceutical composition directly into a tissue region of a patient. In some embodiments, a pharmaceutical composition disclosed herein can be administered parenterally, e.g., by intravenous injection, intracerebroventricular injection, intra-cisterna magna injection, intra-parenchymal injection, or any combination thereof. In some embodiments, a pharmaceutical composition disclosed herein can administered to subject as disclosed herein. In some embodiments, a pharmaceutical composition disclosed herein can administered to human patient. In some embodiments, a pharmaceutical composition disclosed herein can administered to a human patient via at least two administration routes. In some embodiments, the combination of administration routes by be intracerebroventricular injection and intravenous injection; intrathecal injection and intravenous injection; intra-cisterna magna injection and intravenous injection; and/or intra-parenchymal injection and intravenous injection.

In certain embodiments, pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

In certain embodiments, pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen. For injection, the active ingredients of a pharmaceutical composition herein may be formulated in aqueous solutions, preferably in physiologically compatible buffers such as Hank's solution, Ringer's solution, physiological salt buffer, or any combination thereof.

In certain embodiments, pharmaceutical compositions described herein may be formulated for parenteral administration, e.g., by bolus injection or continuous infusion. Formulations for injection herein may be presented in unit dosage form, e.g., in ampoules or in multidose containers with optionally, an added preservative. In some embodiments, compositions herein may be suspensions, solutions or emulsions in oily or aqueous vehicles, and/or may contain formulatory agents such as suspending, stabilizing and/or dispersing agents.

In certain embodiments, pharmaceutical compositions herein formulated for parenteral administration may include aqueous solutions of the active preparation (e.g., septin inhibitors, bleb inhibitors, targeted therapies, or any combination thereof) in water-soluble form. In some embodiments, compositions herein comprising suspensions of the active preparation may be prepared as oily or water-based injection suspensions. Suitable lipophilic solvents and/or vehicles for use herein may include, but are not limited to, fatty oils such as sesame oil, or synthetic fatty acids esters such as ethyl oleate, triglycerides or liposomes. In some embodiments, compositions herein comprising aqueous injection suspensions may contain substances which increase the viscosity of the suspension, such as sodium carboxymethyl cellulose, sorbitol, and/or dextran. In some embodiments, compositions herein comprising a suspension may also contain one or more suitable stabilizers and/or agents which increase the solubility of the active ingredients (e.g., septin inhibitors, bleb inhibitors, targeted therapies, or any combination thereof) to allow for the preparation of highly concentrated solutions.

In some embodiments, compositions herein may comprise the active ingredient in a powder form for constitution with a suitable vehicle, e.g., sterile, pyrogen-free water-based solution, before use.

Pharmaceutical compositions suitable for use in context of the present disclosure may include compositions wherein the active ingredients can be contained in an amount effective to achieve the intended purpose. In some embodiments, a therapeutically effective amount means an amount of active ingredients (e.g., septin inhibitors, bleb inhibitors, targeted therapies, or any combination thereof) effective to prevent, slow, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

In some embodiments, toxicity and therapeutic efficacy of the active ingredients disclosed herein can be determined by standard pharmaceutical procedures in vitro, in cell cultures or experimental animals. In some embodiments, data obtained from these in vitro and cell culture assays and animal studies can be used in formulating a range of dosage for use in a human subject. In some embodiments, a dosage for use herein may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1).

In certain embodiments, dosage amounts and/or dosing intervals may be adjusted individually to brain or blood levels of the active ingredient that are sufficient to induce or suppress the biological effect (minimal effective concentration, MEC). In some embodiments, the MEC for an active ingredient (e.g., septin inhibitors, bleb inhibitors, targeted therapies, or any combination thereof) may vary for each preparation but can be estimated from in vitro data. In some embodiments, dosages necessary to achieve the MEC herein may depend on individual characteristics and route of administration. Detection assays can be used to determine plasma concentrations.

In certain embodiments, depending on the severity and responsiveness of the condition to be treated, dosing with compositions herein can be of a single or a plurality of administrations, with course of treatment lasting from several days to several weeks or until cure is effected or diminution of the disease state is achieved.

In certain embodiments, amounts of a composition herein to be administered will be dependent on the subject being treated, the severity of the affliction, the manner of administration, the judgment of the prescribing physician, and the like. In some embodiments, effective doses may be extrapolated from dose-responsive curves derived from in vitro or in vivo test systems.

### III. Methods of Use

The present disclosure provides for methods of treating, attenuating, and preventing cancer in a subject in need thereof. The present disclosure also provides for methods of impairing tumor growth compared to tumor growth in an untreated subject with identical disease condition and predicted outcome. In certain embodiments, a method for treating, attenuating, or preventing tumor growth or a method for treating, attenuating, or preventing a cancer and/or metastasis in a subject can include administering to a subject, including a human subject, an effective amount of a composition as disclosed herein.

Embodiments of the instant disclosure also provide methods and compositions for treating cancers, tumors, or any combination thereof resistant to or suspected of becoming resistant to one or more anticancer drugs/therapies. As used herein an “anticancer drug” refers to any drug that for the treatment of malignant, or cancerous, disease. Anticancer therapy refers to a treatment regime for the treatment of malignant, or cancerous, disease such as administration of an anticancer drug, radiation, surgical methods, and the like. In certain embodiments, methods disclosed herein may be used treating cancers, tumors, or any combination thereof resistant to or suspected of becoming resistant to one or more targeted therapies (e.g., MAPKi).

In some embodiments, the subject herein may be a human patient having a refractory disease, for example, a refractory melanoma. As used herein, “refractory” refers to the tumor that does not respond to or becomes resistant to a treatment. In some embodiments, the subject herein may have a tumor that is resistant to at least one targeted therapy. In some embodiments, the subject herein may have a tumor that is resistant to a MAPKi, a BRAFi, or a combination thereof. In some embodiments, the subject may be a human patient having a relapsed disease, for example, a relapsed melanoma. As used herein, “relapsed” or “relapses” refers to a tumor that returns or progresses following a period of improvement (e.g., a partial or complete response) with treatment.

A subject having a target solid tumor as disclosed herein, for example, melanoma, can be identified by routine medical examination, e.g., laboratory tests, organ functional tests, genetic tests, interventional procedure (biopsy, surgery) any and all relevant imaging modalities. In some embodiments, a subject to be treated by the methods described herein may have one or more cancers or one or more tumors having at least one somatic mutation. In some aspects, a subject herein may have a cancer and/or tumor with one or more somatic mutations in a BRAF gene, a NRAS gene, a RAS gene, a RAF gene, an EGFR gene, a KRAS gene, or any combination thereof. In some embodiments, the subject to be treated by the methods described herein is a human cancer patient who has undergone or is subjecting to an anti-cancer therapy, for example, chemotherapy, radiotherapy, immunotherapy, or surgery. In some embodiments, a subject shows disease progression through the treatment. In other embodiments, a subject is resistant to the treatment (either de novo or acquired). In some embodiments, such a subject is demonstrated as having advanced malignancies (e.g., inoperable or metastatic). Alternatively, or in addition, in some embodiments, the subject has no standard therapeutic options available or ineligible for standard treatment options, which refer to therapies commonly used in clinical settings for treating the corresponding solid tumor.

In certain embodiments, a tumor to be treated by compositions and methods disclosed herein can be a solid tumor. In some embodiments disclosed herein, compositions and methods disclosed herein are designed to re-sensitize or sensitize a tumor in a subject to a targeted therapy (e.g., one or more MAPKi) to reduce costs, improve outcome and reduce or eliminate patient exposure to an anticancer therapy without significant effect.

In some embodiments, a subject can have an MAPKi resistant tumor or be suspected of developing such a tumor where additional agents can be administered to re-sensitize or sensitize a tumor in a subject where the tumor includes a solid tumor. In some embodiments, a solid tumor can be an abnormal mass of tissue that is devoid of cysts or liquid regions within the tumor. In some embodiments, solid tumors can be benign (not progressed to a cancer), a malignant or metastatic tumor. In some embodiments, a solid tumor herein can be a malignant cancer that has metastasized. In other embodiments, solid tumors contemplated herein can include, but are not limited to, sarcomas, carcinomas, lymphomas, gliomas or any combination thereof. In accordance with some embodiments herein, tumors resistant to platinum-based chemotherapy can include, but are not limited to, a testicular tumor, ovarian tumor, cervical tumor, a kidney tumor, bladder tumor, head-and-neck tumor, liver tumor, stomach tumor, lung tumor, endometrial tumor, esophageal tumor, breast tumor, cervical tumor, central nervous system tumor, germ cell tumor, prostate tumor, Hodgkin's lymphoma, non-Hodgkin's lymphoma, neuroblastoma, sarcoma, multiple myeloma, melanoma, mesothelioma, osteogenic sarcoma or any combination thereof. In some embodiments, a targeted tumor contemplated herein can include a solid tumor such as ovarian tumors, breast tumors, or any combination thereof. In some embodiments, a targeted tumor contemplated herein can include a solid tumor such as lung adenocarcinoma, colon cancer, melanoma, or any combination thereof.

In some embodiments, compositions of use herein can include at least one septin inhibitor and/or bleb inhibitor as disclosed herein. In some other embodiments, a combination therapy herein may include administering to a subject in need at least one targeted therapy and at least one septin inhibitor and/or bleb inhibitor disclosed herein. In certain embodiments, septin inhibitors and/or bleb inhibitors disclosed herein can be administered to a subject alone or in combination with a targeted therapy (e.g., a MAPKi), daily, every other day, twice weekly, every other day, every other week, weekly, monthly, or any other suitable dosing regimen.

In certain embodiments, compositions disclosed herein can treat and/or prevent cancer in a subject in need. In some embodiments, compositions disclosed herein can impair tumor growth compared to tumor growth in an untreated subject with identical disease condition and predicted outcome. In some embodiments, tumor growth can be stopped following treatment with compositions disclosed herein. In other embodiments, tumor growth can be impaired at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In other words, tumors in subject treated using a composition of the disclosure have tumors that grow at least 5% less (or more as described above) when compared to an untreated subject with identical disease condition and predicted outcome. In some embodiments, tumor growth can be impaired at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, tumor growth can be impaired at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated subject with identical disease condition and predicted outcome.

In some embodiments, treatment of tumors with compositions disclosed herein can result in a shrinking of a tumor in comparison to the starting size of the tumor. In some embodiments, tumor shrinking is at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% (meaning that the tumor is completely gone after treatment) compared to the starting size of the tumor.

In certain embodiments, compositions disclosed herein can improve patient life expectancy compared to the cancer life expectancy of an untreated subject with identical disease condition and predicted outcome. As used herein, “patient life expectancy” is defined as the time at which 50 percent of subjects are alive and 50 percent have passed away. In some embodiments, patient life expectancy can be indefinite following treatment with a composition disclosed herein. In other aspects, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In some embodiments, patient life expectancy can be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, patient life expectancy can be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated patient with identical disease condition and predicted outcome.

In some embodiments, the methods of the present disclosure increase anti-tumor activity (e.g., reduce cell proliferation, tumor growth, tumor volume, and/or tumor burden or load or reduce the number of metastatic lesions over time) by at least about 10%, 20%, 25%, 30%, 40%, 50%, 60%, 70%, 75%, 80%, 85%, 90%, 95%, or more as compared to levels prior to treatment or in a control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more. In some embodiments, methods disclosed herein may include administration of the compositions herein to reduce tumor volume, size, load or burden in a subject to an undetectable size, or to less than about 1%, 2%, 5%, 10%, 20%, 25%, 30%, 40%, 50%, 60%, 70%, 75%, 80%, or 90% of the subject's tumor volume, size, load or burden prior to treatment. In other embodiments, methods disclosed herein may include administration of the compositions herein to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment.

In some embodiments, a subject to be treated by any of the methods and/or compositions herein can present with one or more cancerous solid tumors, metastatic nodes, or any combination thereof. In some embodiments, a subject herein may have a cancerous tumor cell source that can be less than about 0.2 cm³ to at least about 20 cm³ or greater, at least about 2 cm³ to at least about 18 cm³ or greater, at least about 3 cm³ to at least about 15 cm³ or greater, at least about 4 cm³ to at least about 12 cm³ or greater, at least about 5 cm³ to at least about 10 cm³ or greater, or at least about 6 cm³ to at least about 8 cm³ or greater.

In certain embodiments, the compositions disclosed herein can be effective for treating at least one tumor cell in a solid tumor from a subject in need. In some embodiments, the amount of viable tumor cells may be reduced by at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome.

Conventional methods, known to those of ordinary skill in the art of medicine, can be used to administer the compositions disclosed herein to a subject, depending upon the type of disease to be treated or the site of the disease. In some embodiments, compositions herein can be administered to a subject by intravenous infusion, by subcutaneous administration, by inhalation, by intranasal administration or other mode of administration. In some embodiments, compositions herein can be administered to a subject orally.

In some embodiments, any of the methods disclosed herein can further include monitoring occurrence of one or more adverse effects in the subject. Exemplary adverse effects include, but are not limited to, hepatic impairment, hematologic toxicity, neurologic toxicity, cutaneous toxicity, gastrointestinal toxicity, or any combination thereof. When one or more adverse effects are observed, the method disclosed herein can further include reducing or increasing the dose of one or more of the disclosed active agents (e.g., septin inhibitor and/or bleb inhibitor), the dose of one or more targeted therapies (e.g., MAPKi), or both depending on the adverse effect or effects in the subject. For example, when a moderate to severe hepatic impairment is observed in a subject after treatment, one or more compositions can be reduced in concentration, frequency of dosing, or a combination thereof.

In some embodiments, one or more disclosed active agents (e.g., septin inhibitor and/or bleb inhibitor) can be administered concurrently with the one or more targeted therapies (e.g., MAPKi) by the same or different modes of administration. In some embodiments, one or more disclosed active agents (e.g., septin inhibitor and/or bleb inhibitor) can be administered before, during or after the one or more targeted therapies (e.g., MAPKi), In other embodiments, the one or more targeted therapies (e.g., MAPKi) can be administered systemically. In certain embodiments, the one or more targeted therapies (e.g., MAPKi) can be administered locally directly to one or more tumors in the subject. In some embodiments, the one or more targeted therapies (e.g., MAPKi) can be administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In other embodiments, the one or more targeted therapies (e.g., MAPKi) can be administered to the subject by intravenous infusion.

An effective amount of the pharmaceutical composition described herein can be administered to a subject (e.g., a human) in need of the treatment via a suitable route, systemically or locally. In some embodiments, one or more disclosed active agents (e.g., septin inhibitor and/or bleb inhibitor) can be administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In some embodiments, one or more disclosed active agents (e.g., septin inhibitor and/or bleb inhibitor) can be administered orally.

In some embodiments, methods herein of treating a cancer with one or more septin inhibitors and/or bleb inhibitors as disclosed herein can further include treating a subject with at least one additional therapeutic regimen, for example, chemotherapy, radiotherapy, immunotherapy, or surgery. In some embodiments, a subject treated with any of the methods herein can have completed an additional therapeutic regimen, be receiving an additional therapeutic regimen, or can receive an additional therapeutic regimen following treatment according to the methods herein.

### IV. Kits

The present disclosure provides kits for use in treating or alleviating cancer and/or a solid tumor described herein. Such kits can include one or more containers including one or more disclosed active agents (e.g., septin inhibitor and/or bleb inhibitor). In some embodiments, kits can include one or more containers including one or more one or more disclosed active agents (e.g., septin inhibitor and/or bleb inhibitor) and one or more targeted therapies described herein (e.g., an MAPKi).

In some embodiments, the kits herein can include instructions for use in accordance with any of the methods described herein. The included instructions can have a description of administration of the one or more disclosed active agents (e.g., septin inhibitor and/or bleb inhibitor), and/or the one or more targeted therapies described herein (e.g., an MAPKi), to treat, delay the onset, or alleviate a target disease as those described herein, or a combination thereof. In some embodiments, the kit can further include a description of selecting an individual suitable for treatment based on identifying whether that individual has the target disease, e.g., applying the diagnostic method as described herein. In still other embodiments, the instructions can have a description of administering any one of the compositions described herein to an individual at risk of the target disease.

In some embodiments, kit instructions relating to the use of one or more disclosed active agents (e.g., septin inhibitor and/or bleb inhibitor), one or more targeted therapies (e.g., an MAPKi) described herein, or a combination thereof can generally include information as to dosage, dosing schedule, and route of administration for the intended treatment. The containers can be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the solid tumor. In some embodiments, instructions are provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, a kit has a sterile access port (for example the container can be an intravenous solution bag or a vial having a stopper pierceable by a hypodermic injection needle). In some embodiments, the container also has a sterile access port (for example the container is an intravenous solution bag or a vial having a stopper pierceable by a hypodermic injection needle). A

In some embodiments, kits herein can optionally provide additional components such as buffers and interpretive information. Normally, the kit comprises a container and a label or package insert(s) on or associated with the container. In some embodiments, the invention provides articles of manufacture comprising contents of the kits described above.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Introduction to Examples 1-7

Attachment-dependent survival is a fundamental aspect of metazoan cell physiology. Near universally, points of attachment-either to the extracellular matrix or other cells-generate pro-survival signals, often through the assembly of ultrastructural cellular architecture such as focal adhesions and adherens junctions. These adhesive structures can operate as signaling hubs by controlling local concentrations of signaling factors, protein scaffolds, and effector proteins. If deprived of attachments, most cells undergo a detachment-induced form of programmed cell death called anoikis. Acquisition of anoikis resistance is a scantily understood, but pivotal step, in cancer disease progression as metastasizing cancer cells often lose firm attachment to surrounding tissue. In these poorly attached states, cells often adopt rounded morphologies and form small hemispherical plasma membrane protrusions called blebs. Bleb function has long been investigated in the context of amoeboid migration but is it has not been adequately examined in human disease mechanisms. Exemplary methods provided in the present disclosure are toward the cell morphological states and blebbing-induced formation of membrane-proximal signaling hubs that initiate signaling cascades leading to anoikis resistance.

### Example 1. Bleb Inhibition Disrupts Anoikis Resistance in Melanoma Cells

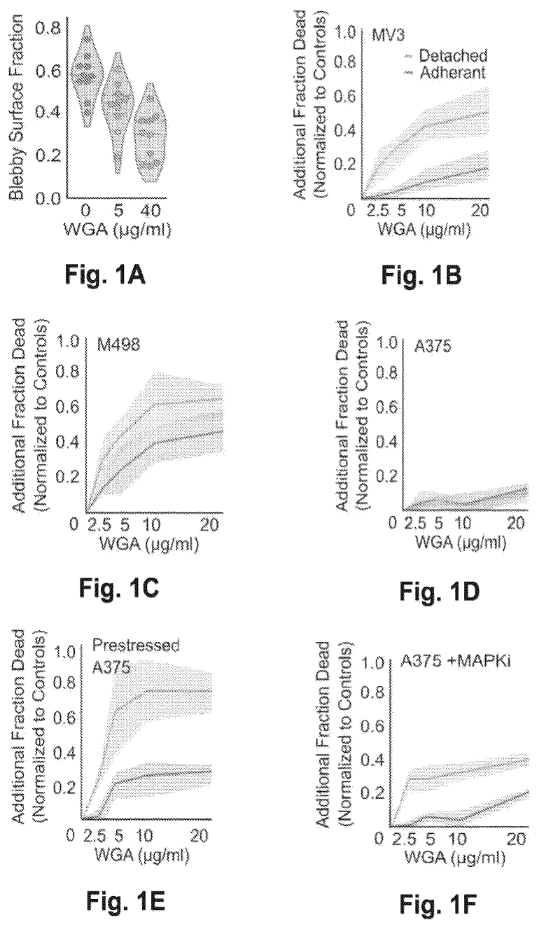

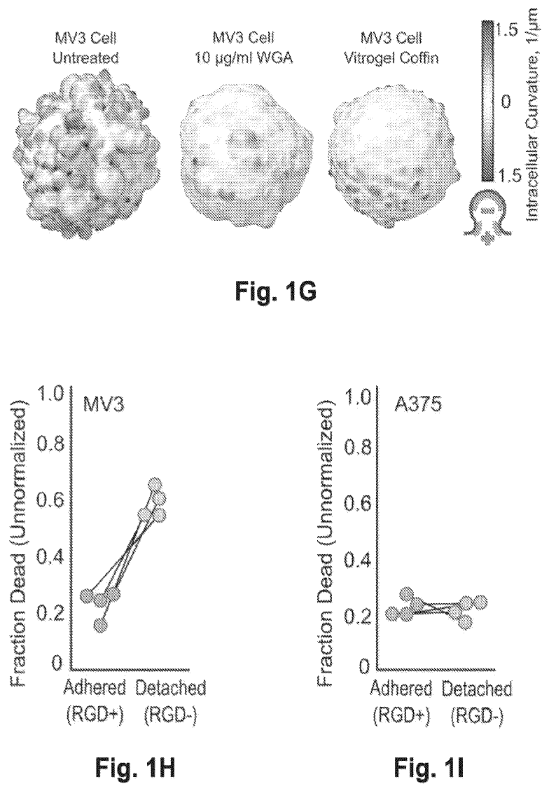

Bleb formation was inhibited using wheat germ agglutinin (WGA), a lectin that reduces plasma membrane deformability by crosslinking components of the glycocalyx. Using MV3 melanoma cells, WGA's effects as a dosage dependent inhibitor of bleb density was confirmed by morphological motif analysis on single cell surfaces (FIG. 1A). To investigate putative bleb function as a driver of anchorage-independent cell survival assays were designed that consistently minimized both cell-substrate and cell-cell attachment over a period of 24 hours. This was accomplished by seeding cells at very low density (approx. 250 or 100 cells/ml) in non-adherent culture dishes under constant gentle agitation. For controls, cells were grown at identical concentrations in adherent variants of the same culture dishes. Together, this generated pairs of ‘detached’ and ‘adhered’ cell populations in paired same-day experiments.

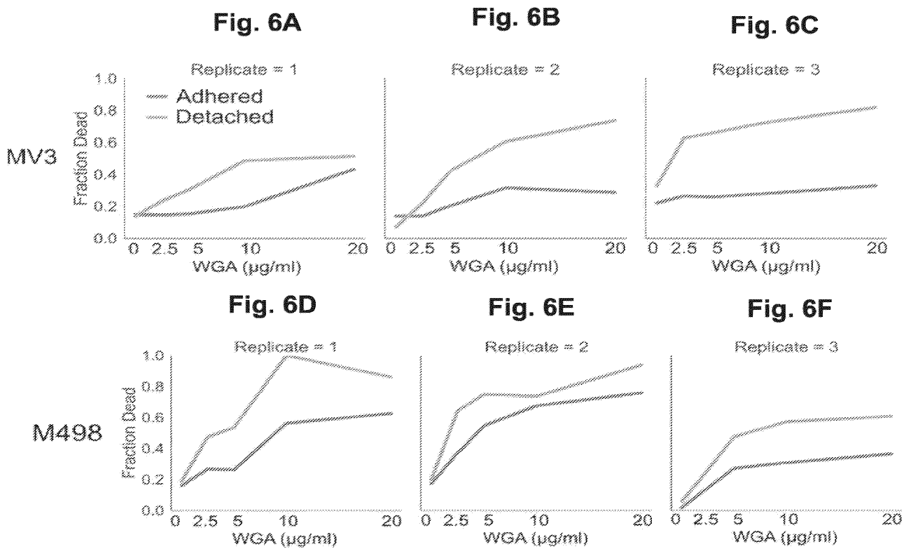

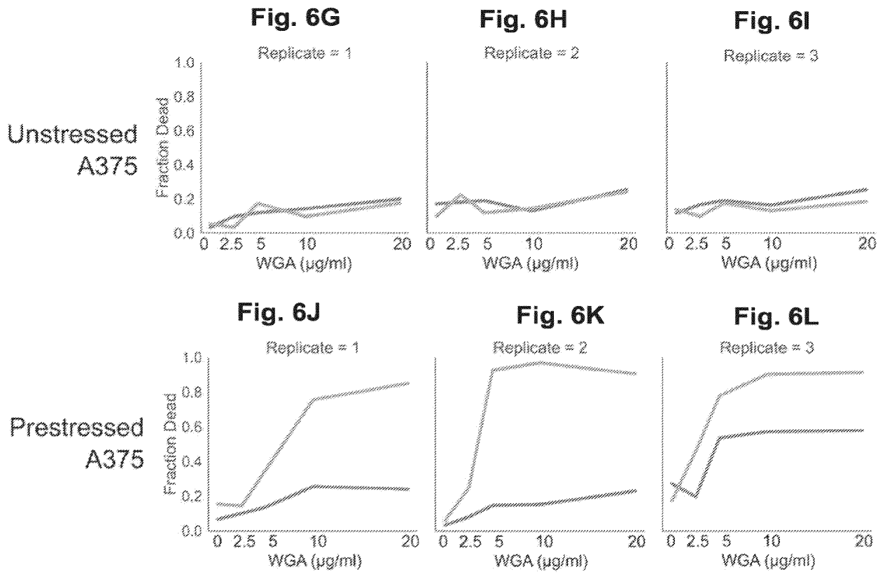

WGA dose response survival analysis was performed on three different melanoma cell lines: MV3 (NRAS (Q61R)-driven), A375 (BRAF (V600E)-driven), and M498 (BRAF (V600E)-driven primary cell line). After 24 hours of bleb inhibition, detached MV3 cells showed high levels of cell death as measured by ethidium homodimer staining, but little to none when adhered (FIGS. 1B-1F). Both adhered and detached M498 cells die upon WGA treatment, but detached cells experience significantly more death, both in aggregate and in individual matched experiments (FIGS. 1B-1F and FIGS. 6A-6L). Sum cell counts for all replicates in ascending order of WGA dosage are provided in Table 1A and Table 1B.

TABLE 1A

| | | | | | Un- | Un- | Pre- | Pre- |
|---|---|---|---|---|---|---|---|---|
| | | | | | stressed | stressed | stressed | stressed |
| | MV3 | MV3 | M498 | M498 | A375 | A375 | A375 | A375 |
| | Detached | Adhered | Detached | Adhered | Detached | Adhered | Detached | Adhered |
| 0 WGA | 172 | 305 | 121 | 112 | 313 | 137 | 242 | 93 |
| 2.5 WGA | 179 | 234 | 107 | 123 | 340 | 162 | 93 | 87 |
| 5 WGA | 151 | 254 | 84 | 103 | 234 | 184 | 54 | 75 |
| 10 WGA | 109 | 136 | 117 | 94 | 376 | 171 | 31 | 79 |
| 20 WGA | 137 | 254 | 69 | 96 | 281 | 181 | 42 | 74 |
| 0 WGA | 104 | 167 | 89 | 161 | 212 | 204 | 69 | 59 |
| 2.5 WGA | 113 | 185 | 104 | 113 | 134 | 138 | 59 | 51 |
| 5 WGA | 123 | 174 | 36 | 137 | 195 | 211 | 99 | 28 |
| 10 WGA | 120 | 139 | 38 | 111 | 131 | 162 | 51 | 42 |
| 20 WGA | 133 | 149 | 33 | 137 | 153 | 173 | 46 | 57 |
| 0 WGA | 194 | 216 | 80 | 132 | 342 | 235 | 185 | 343 |
| 2.5 WGA | 310 | 150 | 80 | 142 | 323 | 153 | 341 | 443 |
| 5 WGA | 205 | 169 | 39 | 107 | 241 | 194 | 496 | 345 |
| 10 WGA | 272 | 151 | 32 | 80 | 340 | 245 | 235 | 383 |
| 20 WGA | 292 | 141 | 31 | 83 | 245 | 201 | 237 | 344 |

TABLE 1B

| | MAPKi | MAPKi |
|---|---|---|
| | A375 | A375 |
| | Detached | Adhered |
| 0 WGA | 24 | 76 |
| 2.5 WGA | 20 | 78 |
| 5 WGA | 27 | 85 |
| 10 WGA | 33 | 101 |
| 20 WGA | 28 | 98 |
| 0 WGA | 104 | 207 |
| 2.5 WGA | 94 | 241 |
| 5 WGA | 103 | 200 |
| 10 WGA | 108 | 225 |
| 20 WGA | 153 | 204 |
| 0 WGA | 48 | 80 |
| 2.5 WGA | 87 | 55 |
| 5 WGA | 110 | 64 |
| 10 WGA | 73 | 89 |
| 20 WGA | 83 | 52 |

Compared to adherent MV3 cells, adherent M498 cells spread poorly and were easily detached with gentle pipetting (FIG. 1J), suggesting poor adhesion even in growth conditions that permit attachment and potentially explaining the increased susceptibility to bleb inhibition in adherent conditions. A375 cells were unaffected by WGA treatment, regardless of their attachment state (FIGS. 1B-1F) indicating that bleb inhibition was not a universal mechanism for establishing anoikis resistance. However, it was observed that the confluency of A375 cells prior to these viability experiments influenced outcomes, with cells from more confluent culture conditions experiencing more death upon WGA treatment. To explore this, A375 cells were allowed to remain overconfluent for 48 hours (changing culture media daily) before performing viability experiments as before. It was found that anoikis resistance in these “pre-stressed” A375 cells was strongly dependent upon blebbing, with an effect size even larger than that seen in MV3 cells (FIGS. 1B-1F). Having established that A375 cells can be made reliant on blebbing by certain stress conditions, the viability assay was also applied also to experiments in which A375 cells were challenged by direct pharmacological stress. Specifically, the effect of bleb inhibition was tested on A375 cells pre-incubated for 48 hours in media containing 10 nM Dabrafenib (targeting BRAF (V600E)) and 1 nM Trametinib (targeting Mek1/2), a combination used as a frontline therapy for Braf-mutated tumors. Drug-treated A375 cells experienced increased cell death upon WGA treatment when detached and minimal change in viability when adhered (FIG. 1B-1F).

To ensure that the WGA-mediated disruption of anoikis resistance was due to bleb inhibition and not an off-target effect of WGA, a complementary experiment was performed using VitroGel, an abiotic hydrogel used in cell culture, to form bleb-restricting “coffins” around individual cells (FIG. 1G). Coffins for “detached” cell growth were made using non-adherent VitroGel, while adherent coffins were made using VitroGel-RGD, which contained integrin-binding RGD domains that allow for cell adhesion. These experiments were performed on WGA-sensitive MV3 cells and WGA-insensitive A375 cells. Far more MV3 cells died in non-adherent than in adherent VitroGel coffins, whereas A375 cells showed no difference in viability between the two conditions (FIGS. 1H-11). Sum cell counts for all replicates of FIGS. 1H-11 are provided in Table 2.

TABLE 2

| | MV3 | MV3 | A375 | A375 |
|---|---|---|---|---|
| | Detached | Adhered | Detached | Adhered |
| Replicate 1 | 94 | 194 | 152 | 131 |
| Replicate 2 | 87 | 114 | 113 | 132 |
| Replicate 3 | 91 | 33 | 89 | 108 |
| Replicate 4 | 95 | 48 | 107 | 138 |

Taken together, these results indicated that dynamic blebbing contributed to anoikis resistance both in NRAS- and in BRAF-mutated melanoma, suggesting that this cell morphological program is a broadly adopted survival strategy in melanoma cells. Moreover, the observation in FIG. 1F of MAPK inhibition (MAPKi) via Dabrafenib/Trametinib sensitizing BRAF (V600E)-driven cells to bleb inhibition suggests that bleb-formation serves also as an acute defense mechanism against drug attacks. In other words, dabrafenib/trametinib treatment phenocopied the “prestressed” state allowing treated cells to become vulnerable to bleb inhibition. These data suggests that BRAF (V600E) melanoma cells begin relying on bleb signaling after BRAF and downstream pathways have been inhibited, meaning that bleb signaling may be a key player in melanoma drug resistance.

### Example 2. Bleb-Generated Plasma Membrane Curvature Drives the Formation of Cortical Septin Structures

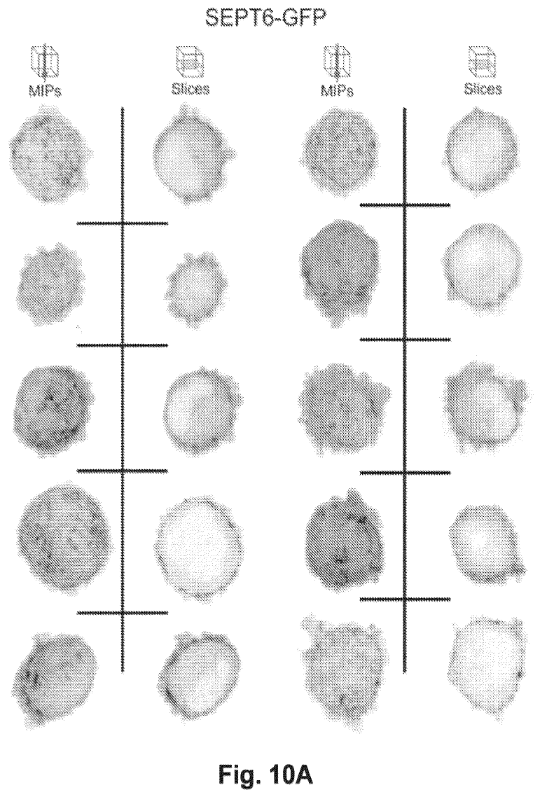

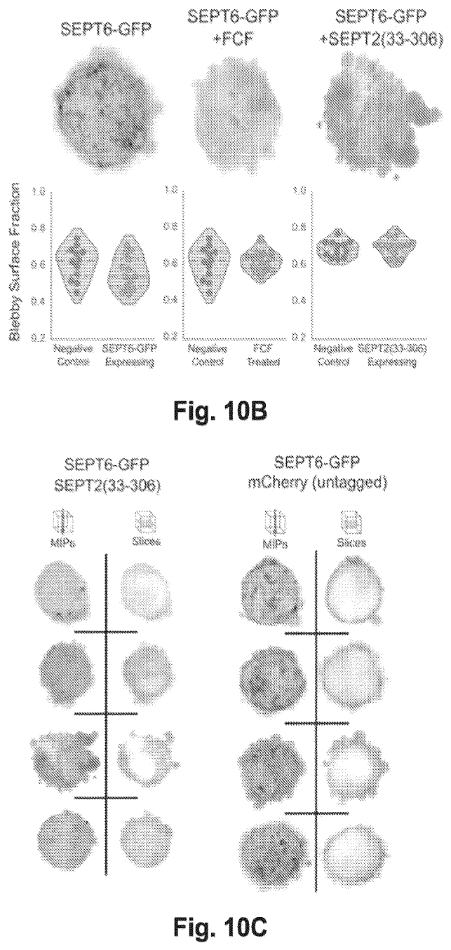

A feature of blebbing vs bleb-inhibited cells is the significant enrichment of micron-scale plasma membrane curvature. For an intracellular observer, curvature is negative/concave in the bleb proper (reaching values in the range of κ=−1.5 μm-1, where K represents the inverse of the radius of curvature) and positive/convex between the blebs (reaching values in the range of κ=1.5 μm-1) (FIG. 1G). A marked recruitment of septins was serendipitously discovered herein to locations in between densely packed blebs in MV3 cells, expressing murine SEPT6-GFP (FIG. 10A). In eukaryotic cells, members of the septin cytoskeletal family are the only proteins known to recognize positive micron-scale membrane curvature that blebbing creates; however, there are currently no reports of an explicit connection between bleb-related surface curvature and septin recruitment. Septins are known to scaffold and regulate factors from diverse signaling pathways, including pro-survival pathways in cancer cells such as, but not limited to, Hypoxia-Inducible Factor (HIF)-1, Epidermal Growth Factor Receptor (EGFR), Mesenchymal Epithelial Transition Factor Receptor (MET), c-Jun NH2-terminal kinase (JNK), and Human Epidermal Growth Factor Receptor 2 (HER2). As such, methods herein assessed whether bleb-dependent anoikis resistance could be achieved through recruitment of curvature-sensitive septin proteins to the plasma membrane via bleb-generated curvature, where they promote pro-survival signaling.

To begin testing such involvement of septins as translators of cell morphology into survival signaling, mass spectrometry of MV3 cell lysate was performed and robust expression of several septin proteins was found (Table 3).

TABLE 3

| | | % of total | | % of total | |
|---|---|---|---|---|---|
| | | septin abundance | | protein abundance | |
| | | (fold change | | (fold change | |
| | | between lysate | | between lysate | |
| Experiment | Septin | and pulldown) | | and pulldown) | |
| MV3 | SEPTIN2 | 19.99 | | 0.04 | |
| Lysate | SEPTIN6 | 40.98 | | 0.09 | |
| | SEPTIN7 | 8.80 | | 0.02 | |
| | SEPTIN9 | 12.93 | | 0.03 | |
| | SEPTIN8 | 0.31 | | <0.01 | |
| | SEPTIN10 | 16.97 | | 0.04 | |
| | SEPTIN11 | ND | | ND | |
| | SEPTIN3 | ND | | ND | |
| | All Septins | N/A | | 0.22 | |
| MV3 | SEPTIN2 | 30.09 | (1.50) | 2.43 | (60.75) |
| SEPT6-HALO | SEPTIN6 | 12.82 | (−3.20) | 1.03 | (11.44) |
| Pulldown | SEPTIN7 | 29.88 | (3.39) | 2.41 | (120.50) |
| | SEPTIN9 | 22.97 | (1.78) | 1.85 | (61.67) |
| | SEPTIN8 | 0.09 | (−3.44) | <0.01 | (N/A) |
| | SEPTIN10 | 3.19 | (−5.32) | 0.28 | (7) |
| | SEPTIN11 | 0.94 | (N/A) | 0.08 | (N/A) |
| | SEPTIN3 | ND | (N/A) | ND | (N/A) |
| | All Septins | N/A | | 8.06 | (35.86) |
| A375 | SEPTIN2 | 26.42 | | 0.03 | |
| Lysate | SEPTIN6 | 31.71 | | 0.03 | |
| | SEPTIN7 | 15.60 | | 0.02 | |
| | SEPTIN9 | 18.99 | | 0.02 | |
| | SEPTIN8 | ND | | ND | |
| | SEPTIN10 | 1.92 | | <0.01 | |
| | SEPTIN11 | 5.36 | | <0.01 | |
| | SEPTIN3 | ND | | ND | |
| | All Septins | N/A | | 0.12 | |
| A375 | SEPTIN2 | 25.71 | (−1.03) | 1.81 | (60.33) |
| SEPT6-HALO | SEPTIN6 | 23.77 | (−1.33) | 1.67 | (55.67) |
| Pulldown | SEPTIN7 | 21.07 | (1.35) | 1.48 | (74) |
| | SEPTIN9 | 22.08 | (1.62) | 1.55 | (77.5) |
| | SEPTIN8 | 0.02 | (N/A) | <0.01 | (N/A) |
| | SEPTIN10 | 7.23 | (3.77) | 0.51 | (N/A) |
| | SEPTIN11 | 0.12 | (−44.67) | <0.01 | (N/A) |
| | SEPTIN3 | ND | (N/A) | ND | (N/A) |

| | | | |
|---|---|---|---|
| All Septins | N/A | 7.04 | (56.02) |

Next, septin structures were visualized in living cells by ectopic expression of mouse SEPT6-GFP and proper integration of the fluorescent probe into native septin complexes was confirmed by pulldowns of mouse SEPT6-HALO. Mass spectrometry showed that all endogenous septins expressed in MV3 cells were pulled down by SEPT6-HALO, at levels 36× higher than in control pulldowns with no SEPT6-HALO expression (septins accounted for 0.22% of total protein abundance in the control and 8.1% in SEPT6-HALO pulldown), suggesting that the probe efficiently forms oligomers with endogenous septins (Table 3).

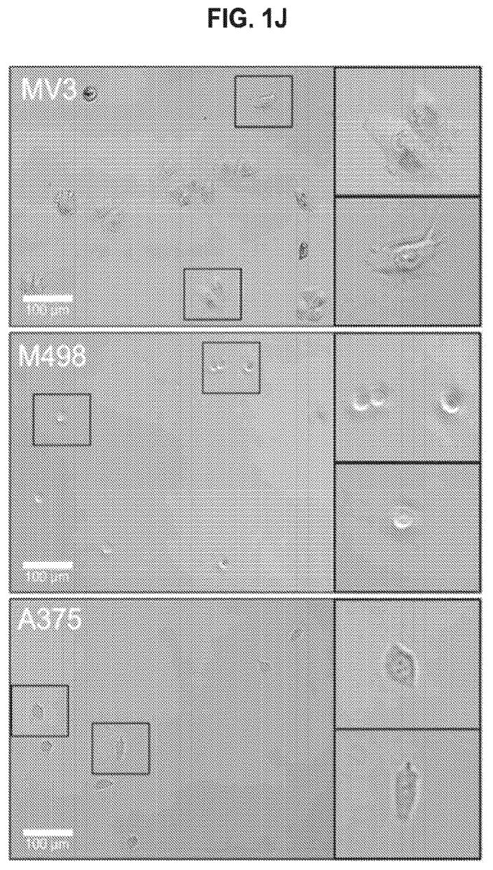

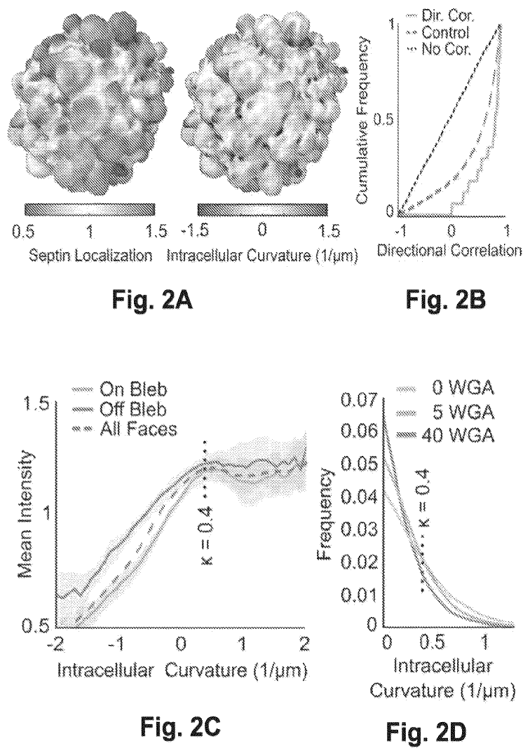

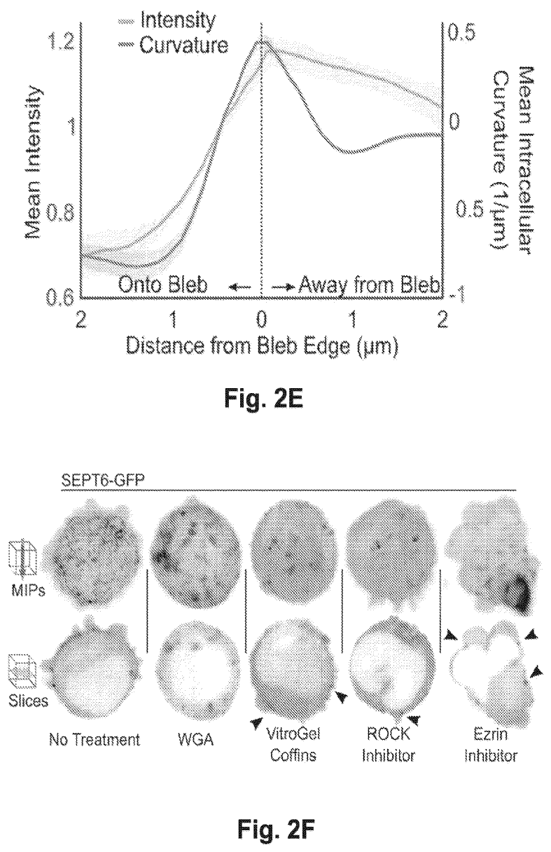

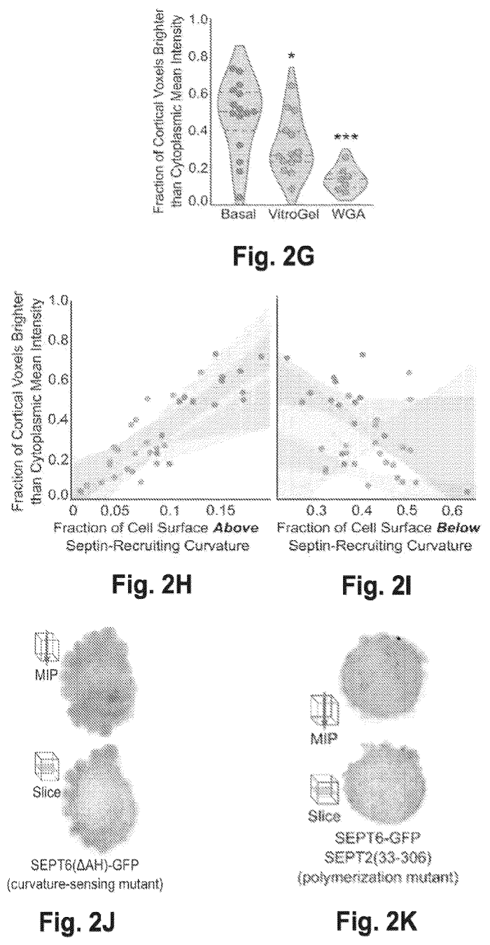

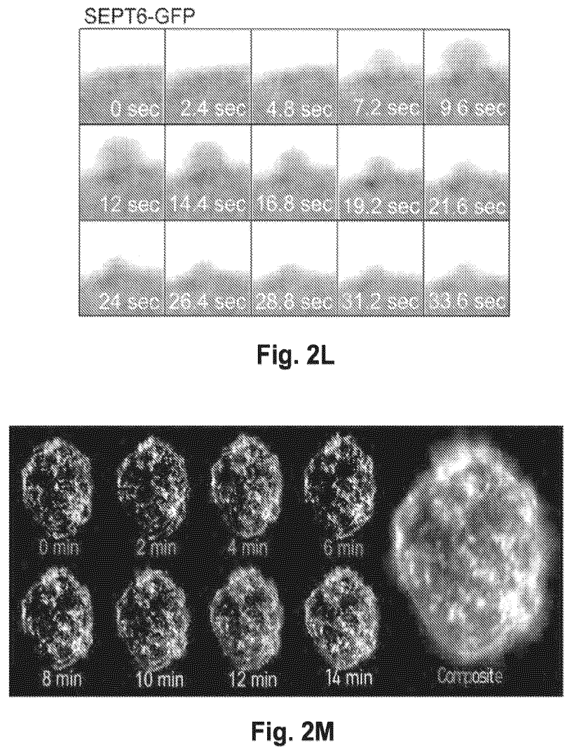

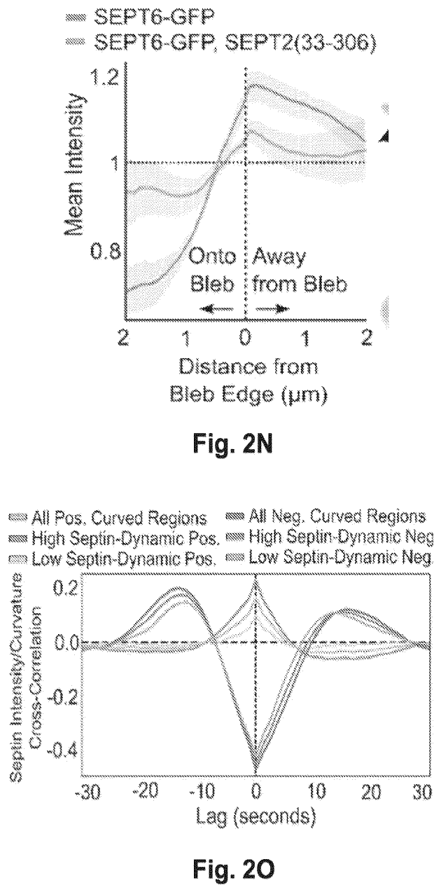

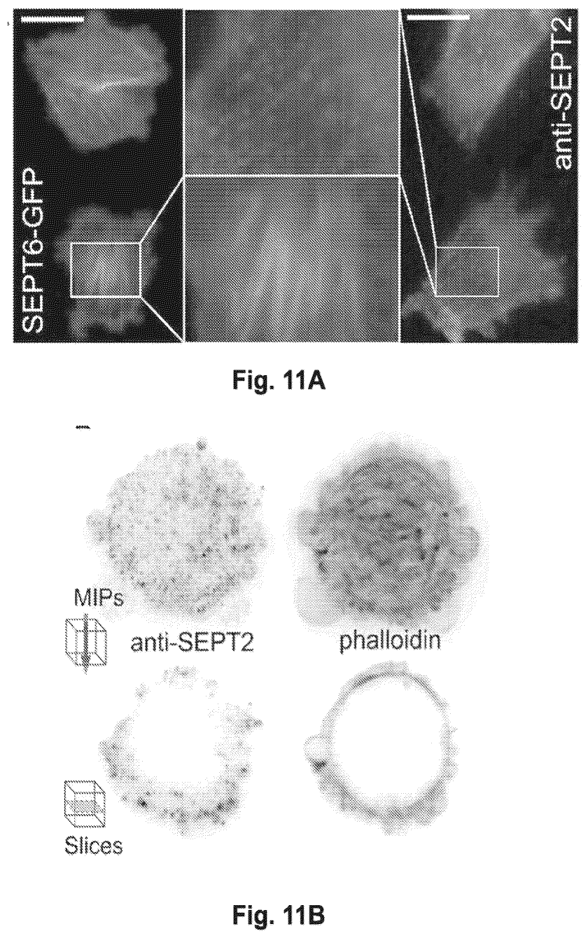

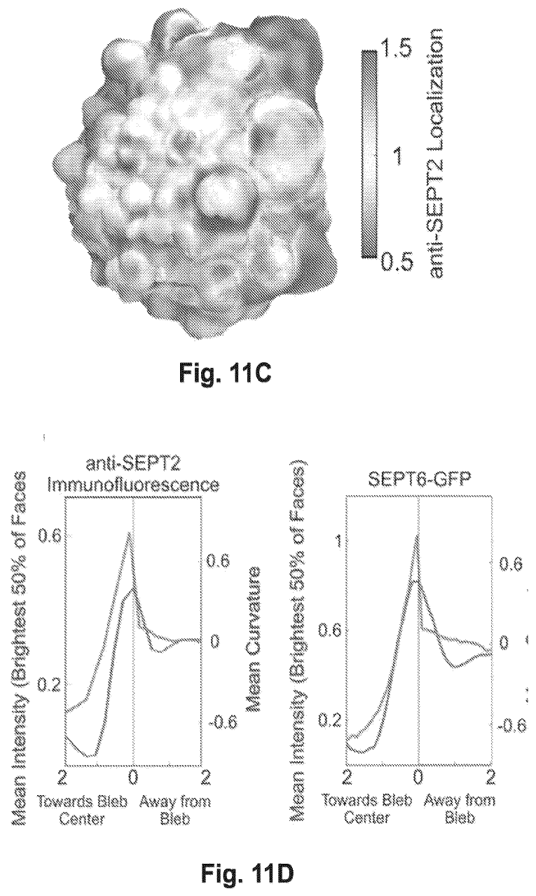

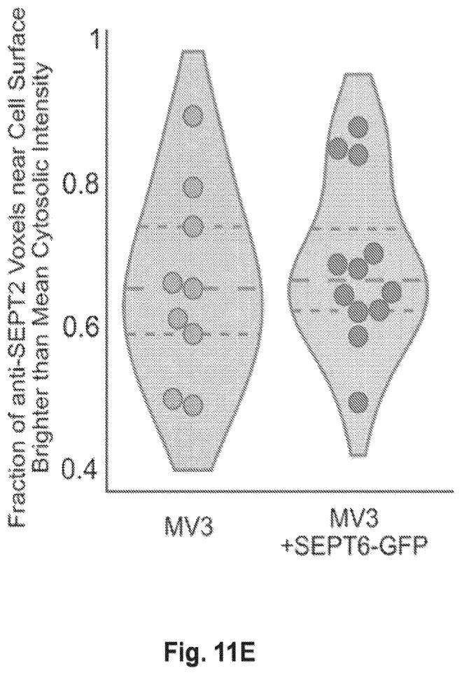

Cell surface renderings of SEPT6-GFP indicated that septin structures were primarily found near blebby cell surface regions, while less blebby regions have few-to-none (FIG. 2A). Computer vision analysis of multiple MV3 cells were imaged at isotropic resolution to accurately measure surface curvature and signal intensity in three dimensions confirmed the observation shown in FIG. 2A, illustrating that bleb and septin polarity vectors were directionally correlated (FIG. 2B). A global examination of these same cell surfaces showed that septins were enriched at positive curvatures above K=0.4 μm-1 (septins have been shown to have high affinity for membrane with curvature values >0.4 μm-1) and drop sharply at lower curvatures (FIG. 2C). Analysis of control and WGA-treated MV3 cells also confirmed that bleb inhibition reduced mean plasma membrane curvature above K=0.4 μm-1 (FIG. 2D). Examining septin structures relative to blebs showed that they tend to be found near bleb edges, with average intensity falling steeply as one moved towards the center of the bleb, and more slowly as one moved away from the bleb (FIG. 2E). The curvature followed a similar pattern, tending to be highest at bleb edges (FIG. 2E). To ensure that these bleb-associated septin structures are not artifacts of SEPT6-GFP expression, analyses was repeated on MV3 cells that lacked the construct using immunofluorescence. It was found that signal from an anti-SEPT2 antibody was enriched at the cell surface near blebs (FIG. 11A-11C), localized to the curvy bases of blebs (FIG. 11D), and importantly, that SEPT6-GFP expression did not alter anti-SEPT2 enrichment near the surface (FIG. 11E).

To more thoroughly test the notion that bleb formation as a morphodynamic process causally drives the assembly of septin structures, blebbing was perturbed by a variety of methods. In addition to WGA and VitroGel, the ROCK inhibitor H1152 (reduces bleb formation by lowering intracellular pressure) and the Ezrin-inhibitor NSC668394 (causes large-scale membrane-cortex detachment and thus substitutes single-micron scale, dynamic blebs with large, stable blebs) were used. Every approach had pronounced effects on septin localization, reducing cortical levels with remaining foci tending to be confined to high curvature areas (FIG. 2F).

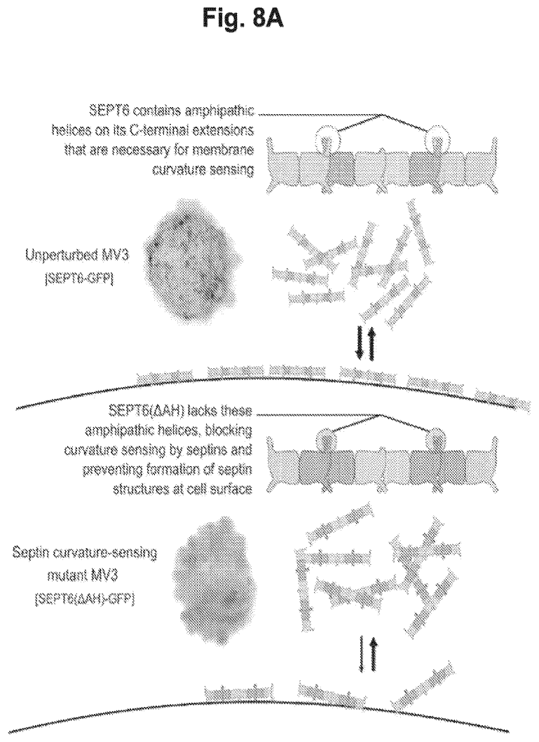

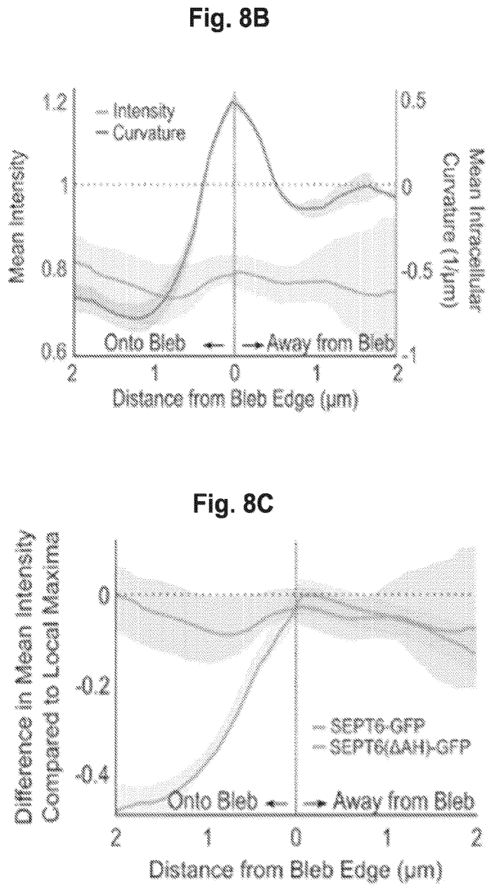

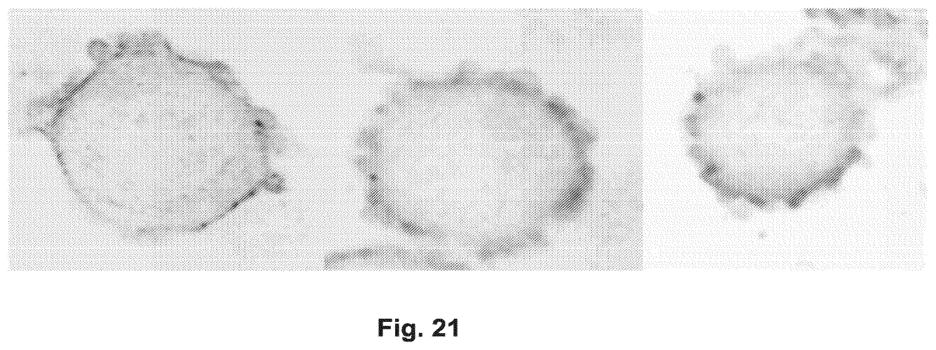

The cortical septin levels were then quantified as the surface fraction of cortical voxels (voxels <0.96 μm from the surface) above mean cytoplasmic SEPT6-GFP intensity. Both WGA and VitroGel treatments significantly reduced cortical septin levels relative control conditions (FIG. 2G). Moreover, in all three experimental conditions, a strong linear relationship was found at the single cell level between the surface fraction of predicted septin-recruiting curvature (K≥0.4 μm-1) and cortical septin levels (FIG. 2H). In contrast, expressing these same data as a function of surface fraction with positive curvature below the septin-recruiting threshold (0 μm 1≥K ≥0.4 μm 1) failed to show a similar relationship (FIG. 21). Thus, septin levels at the cortex universally correlated with the presence of septin-recruiting plasma membrane curvature, and experimental conditions which reduced such curvature, like those inhibiting bleb formation, abrogate septin recruitment. These data provided an example of cell shape dictating subcellular molecular organization. To exclude the possibility that septins localize to bleb edges through a process independent of membrane curvature sensing, mutants of the SEPT6-GFP construct lacking the C-terminal amphipathic helix necessary for curvature sensing by septins were generated as described in Cannon, K. S., et al., J. Cell Biol. 218, 1128-1137 (2019) and Lobato-Márquez, D. et al. Nat. Commun. 2021 121 12, 1-14 (2021), which are each incorporated herein by reference in their entirety (FIG. 8A). The SEPT6 (AAH)-GFP construct contains a GFP tag and comprises a curvature-sensing domain of SEPT6 that was mutated. Cells expressing the SEPT6 (ΔAH)-GFP construct demonstrated completely disrupted cortical septin localization, in contrast to WT SEPT6-GFP (FIG. 2J, FIG. 8B-8C). This demonstrates that the observed cortical septin structures depend entirely on septins' ability to sense the micron-scale membrane curvature associated with bleb formation and further supportthe idea that septin localization in cells is driven by bleb-generated curvature. Similar experiments were performed with a septin polymerization mutant (SEPT6 (33-306)-GFP) showing septin localization in MV3 melanoma cells (FIG. 2K). These studies demonstrated that stable septin structures were polymerization dependent.

### Example 3. Cortical Septin Structures were Formed by Repeated Bleb-Driven Curvature Events

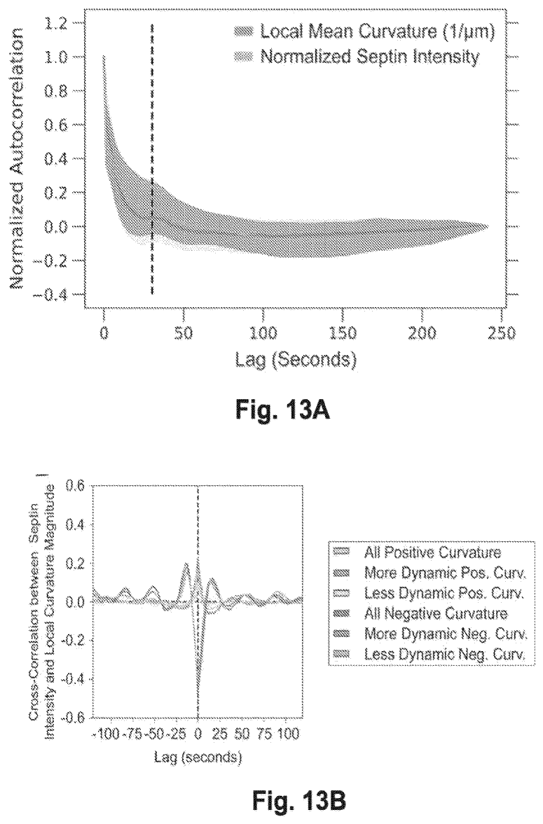

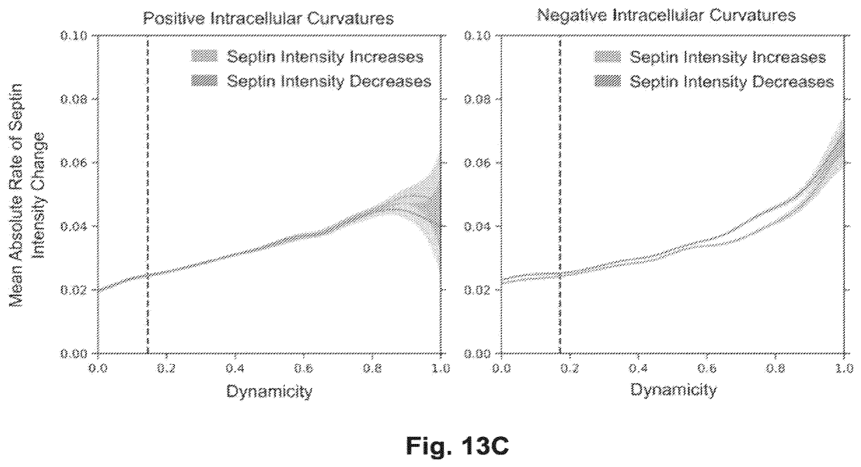

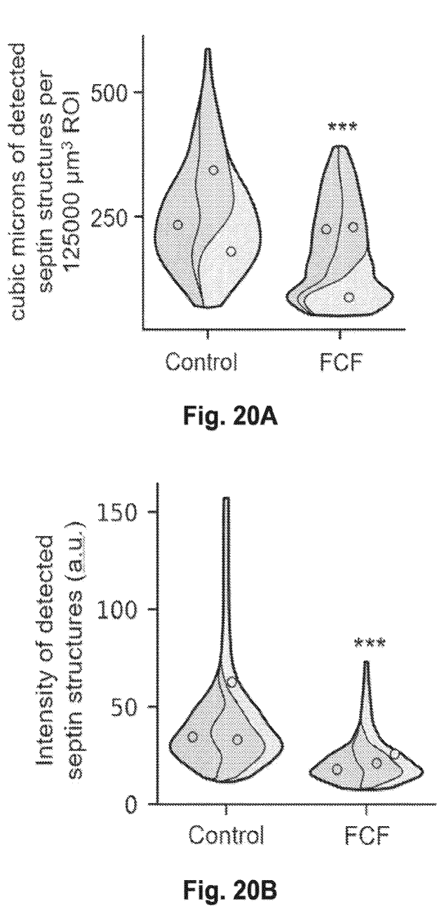

Though bleb lifetimes were typically <60 seconds, cortical septin structures were found to be relatively stable over a period of 14 minutes (FIG. 2M), despite bleb lifetimes typically falling below 60 seconds. This posed the question of how a relatively brief morphodynamic event can determine molecular architecture on longer time scales. High-speed time-lapse light-sheet microscopy was used to address this question. This approach revealed that while “pulses” of septin accumulation were be seen at the curvy bases of blebs, they faded quickly once the bleb was resolved and curvature subsides (FIG. 2L). To confirm this observation, experiments computationally identified regions of the surface with high septin intensity that maintained either positive or negative curvature for at least 25 seconds (the average bleb lifetime, estimated by curvature autocorrelation (FIG. 13A). Within these “contigs” cross-correlation analysis between septin intensity and curvature magnitude showed positive correlation in the positively curved dataset, whereas the negatively curved dataset produced the opposite effect (FIG. 20, FIG. 13B). Interestingly, when contigs were subdivided based on septin intensity dynamics, it was found that more stable regions were less sensitive to positive curvature than dynamic regions, this in contrast to negative contigs, which maintained the same septin/curvature relationship regardless of dynamicity. Examining the mean rates of septin intensity gain or loss within individual contigs as a function of dynamicity showed increased rates of change within more dynamic regions (FIG. 13C), meaning that the areas in which septin concentration shifts more frequently, those shifts also occur at faster rates. This suggests the presence of two septin populations at the cell surface: dynamic pools dependent upon positive curvature, and more stable pools that are less reliant on specific local curvature profiles

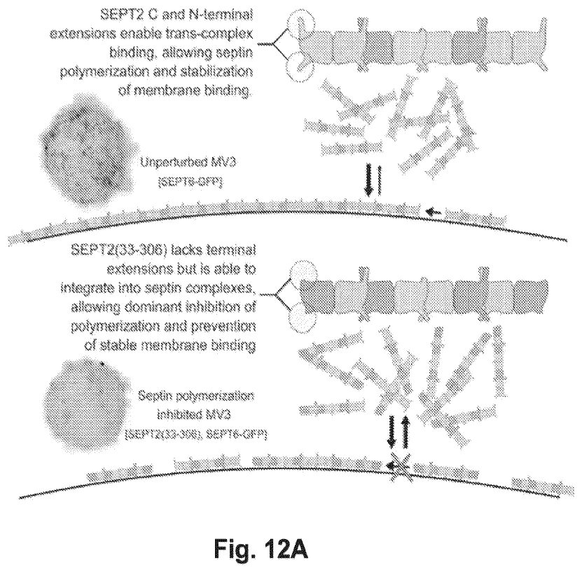

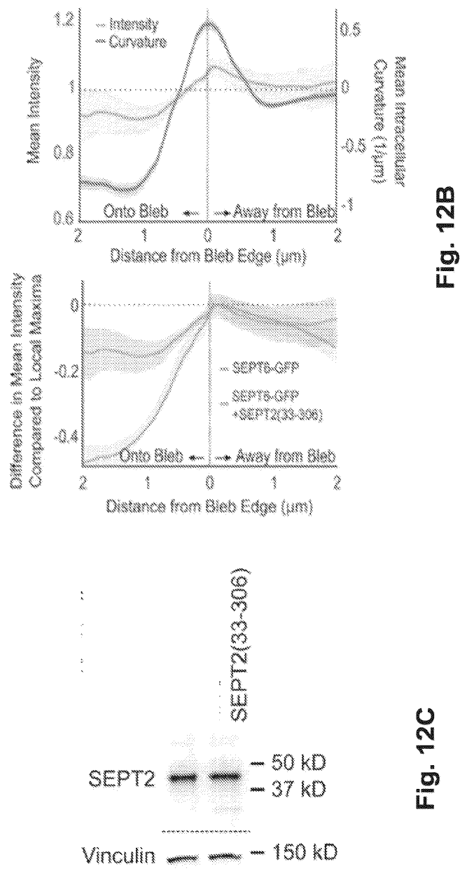

Cytosolic septins predominantly exist as small hexameric or octameric oligomers that preferentially bind to curved membrane, though they quickly detach unless membrane binding is stabilized by local concentration becoming high enough to promote polymerization. To determine if stable cortical septin structures were formed not by individual, but iterative bleb-driven curvature events that resulted in local septin levels surpassing the threshold of polymerization and enabling higher-order structure formation, high-speed time-lapse data was collected in cells expressing septin mutants. To test this, the SEPT2 mutant SEPT2 (33-306) was engineered, which is capable of integrating into septin oligomers but lacks the N- and C-terminal domains necessary for inter-oligomeric polymerization as described in Sirajuddin, M., et al., Proc. Natl. Acad. Sci. U.S.A 106, 16592-16597 (2009), incorporated herein by reference in its entirety (FIG. 12A). When gross septin localization was observed in MV3 cells expressing SEPT2 (33-306), it was found that septin enrichment at the surface was strongly inhibited in a dominant negative fashion (FIG. 2K, FIG. 10C, and FIG. 12B-12C). Bleb-adjacent septin localization still showed a sharp peak at the curvy bleb edge, indicating a continuation of curvature-sensing septin pulses, but moving away from the bleb the signal decayed rapidly and to lower levels compared to the signal profiles in unperturbed cells (FIG. 2N). Conversely, the signal decay was slowed moving towards the center of blebs, matching the decay rate in the opposite direction, and the on-bleb signal was significantly increased. This altered bleb-adjacent localization supports a requirement for inter-oligomer polymerization in the formation of curvature-independent stable septin structures and suggests polymerized septin structures have an increased affinity for non-bleb plasma membrane. This bleb exclusion might be driven by polymerization-dependent crosslinking of the plasma membrane and actomyosin cortex by septins and associated factors.

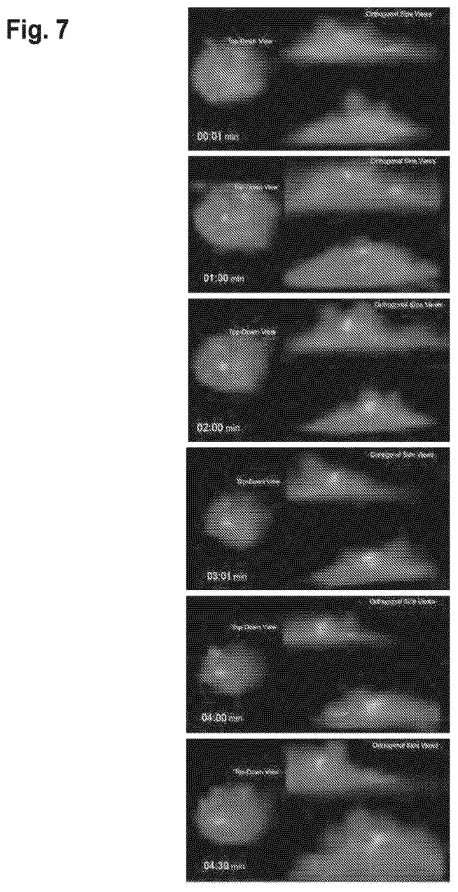

In brief, expression of a wild type septin monitored by high-speed time-lapse data showed stable septin structures emerging only when several pulses occurred in close proximity, resulting in their coalescence into bright, long-lived structures whose intensity does not seem to rely on local curvature profiles (FIG. 7). Taken together, these findings suggest that stable cortical septin structures are formed by iterative bleb-driven curvature events that result in local levels of septin oligomers surpassing the threshold necessary for inter-oligomer polymerization and enabling stabilization through formation of higher-order structures. This would mean that septins are acting as a discrete time integrator of a sustained and spatially persistent dynamic bleb formation process, while also efficiently eliminating the effects of random and isolated morphological events as a discrete spatiotemporal high-pass filter. Thus, septins were suited to translate significant cell morphological cues into cellular signals.

### Example 4. Septins were Necessary for Bleb-Dependent Anoikis Resistance

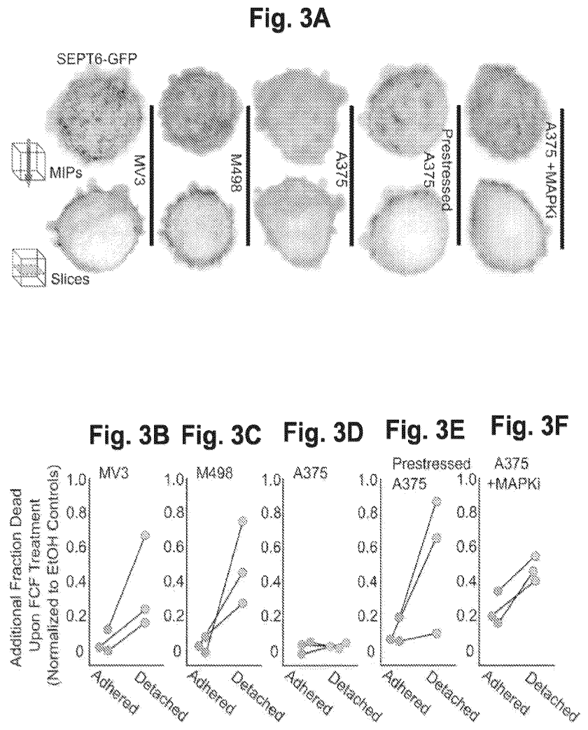

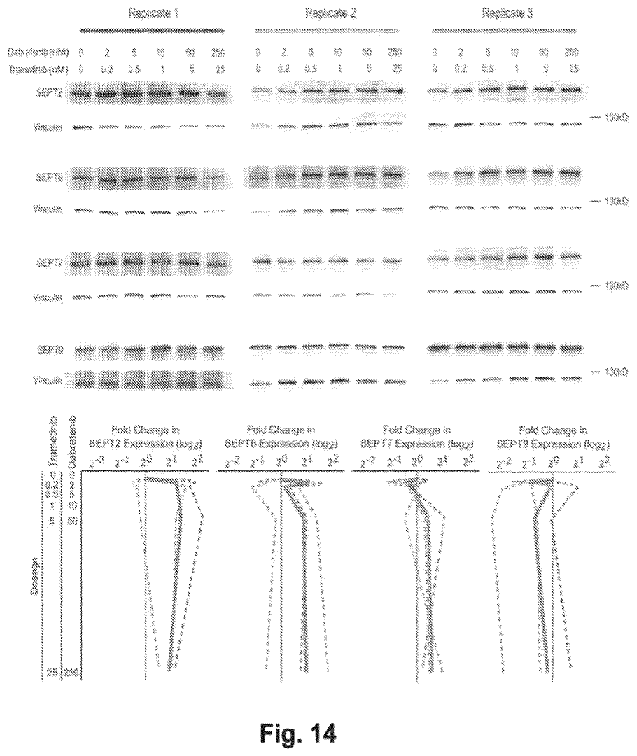

Given the relationship between bleb formation and septin localization observed in the examples herein, it was next sought to determine whether a link existed between such cortical septin structures and bleb-dependent anoikis resistance. Live cell imaging of poorly adhered cells embedded in soft collagen showed that MV3, M498, pre-stressed A375 cells, and MAPKi treated A375 cells possessed extensive cortical septin structures that seem to be spatially associated with blebs (FIG. 3A). In contrast, unperturbed A375 cells, which had shown bleb independent anoikis resistance (FIG. 1D), displayed no septin enrichment at the cortex despite robust bleb formation. Pull-down mass spectrometry experiments described in the examples herein were repeated in unperturbed A375 cells and it was confirmed that the apparent lack of cortical septins was not due to a failure of the probe to integrate into native septin structures (Table 3). Moreover, SEPT6-GFP pulldown and whole-lysate mass spec datasets showed no major differences in septin expression patterns or oligomer subunit ratios between MV3 and unperturbed A375 cells (Table 3). Western blot analysis were performed of MAPKi dose-dependent expression shifts in SEPT2, SEPT6, SEPT7, and SEPT9. No consistent changes in expression or appearance of bands indicating expression of alternate isoforms could be detected (FIG. 14). Hence, the “septin activation” upon prestress or MAPKi is not driven by changes in septin expression but is post-translationally regulated. Of note, SEPT6 did not pull down BORG proteins in MV3 nor A375 cells. These proteins are a family of septin effector proteins known to regulate higher-order polymerization. Thus, the cell lines whose anoikis resistance was bleb-dependent possessed bleb-associated septin structures, while the bleb-independent cell line had none.

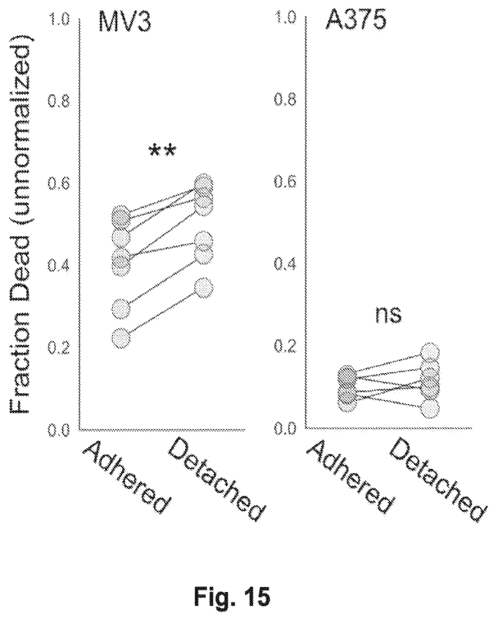

To further test whether bleb-associated septin structures regulated survival signaling, the anoikis-resistance of these four cell lines was measured when treated with the septin inhibitor forchlorfenuron (FCF). FCF treatment resulted in a near-complete ablation of cortical septin structures without altering blebbiness (FIG. 10B) and greatly disrupted anoikis resistance for MV3, M498, and pre-stressed A375 cells while having no appreciable effect on non-stressed A375, mirroring bleb inhibition results (FIGS. 3B-3F). To ensure that these results were not due to off-target effects of FCF an orthogonal experiment was performed using FCF-sensitive MV3 cells and FCF-insensitive A375 cells, inhibiting septin assembly with the dominant negative SEPT2 (33-306) mutant. Like FCF, expression of this construct has no effect on blebbiness (FIG. 10B). As with the FCF treatments, genetic inhibition of septins had no effect on A375 cell survival regardless of adhesion state, while MV3 cells experienced significant death which worsened with detachment (FIG. 15). Taken together, these data suggested that the mechanism allowing blebs to confer anoikis resistance upon melanoma cells depended on bleb-adjacent cortical septin structures. Sum cell counts for all replicates depicted in FIGS. 3B-3F are provided in Table 4A and Table 4B.

TABLE 4A

| | | | | | Un- | Un- | Pre- | Pre- |
|---|---|---|---|---|---|---|---|---|
| | | | | | stressed | stressed | stressed | stressed |
| | MV3 | MV3 | M498 | M498 | A375 | A375 | A375 | A375 |
| | Detached | Adhered | Detached | Adhered | Detached | Adhered | Detached | Adhered |
| Experimental 1 | 170 | 241 | 81 | 111 | 277 | 184 | 345 | 66 |
| Control 1 | 194 | 216 | 80 | 132 | 313 | 137 | 230 | 53 |
| Experimental 2 | 133 | 149 | 100 | 132 | 205 | 156 | 39 | 70 |
| Control 2 | 104 | 167 | 89 | 161 | 212 | 204 | 242 | 93 |
| Experimental 3 | 137 | 254 | 117 | 95 | 341 | 222 | 34 | 33 |
| Control 3 | 172 | 305 | 121 | 112 | 342 | 235 | 69 | 59 |

TABLE 4B

| | MAPKiA | MAPKi |
|---|---|---|
| | A375 | A375 |
| | Detached | Adhered |
| Experimental 1 | 109 | 245 |
| Control 1 | 96 | 364 |
| Experimental 2 | 93 | 192 |
| Control 2 | 104 | 207 |
| Experimental 3 | 29 | 79 |
| Control 3 | 31 | 131 |

### Example 5. Bleb-Associated Cortical Septins Interact with NRAS and MAPK Effector Proteins

Based on the data from the exemplary methods herein, it was hypothesized that bleb-induced septin structures promoted anoikis resistance as a signaling scaffold that amplified survival signals. To identify signaling candidates, BioID proximity labeling was performed using SEPT6 as bait. In brief, 321 septin-interacting proteins were obtained and among them were three plasma membrane-localized components of major signal transduction pathways: Notch, CD44, and NRAS. Also found were Prohibitin, 14-3-37, and Nucleolin among the BioID prey, which are all potent effectors of the RAS/RAF/MEK/ERK and RAS/PI3K/AKT pathways. Reexamination of the previous pulldown data described in the examples herein revealed that NRAS interacted with SEPT6 in MV3 cells, and pre-stresed A375, but not in unperturbed A375, indicating that the NRAS/septin interaction depended on the assembly of bleb-nucleated septin structures. NRAS was an especially intriguing candidate as MV3 cells harbor a constitutively active Q61R mutant, leaving open the possibility that suspended NRAS-driven melanoma cells harness their mutational profile via an acute morphological program to empower oncogenic signaling through the RAS/RAF/MEK/ERK and RAS/PI3K/AKT axes.

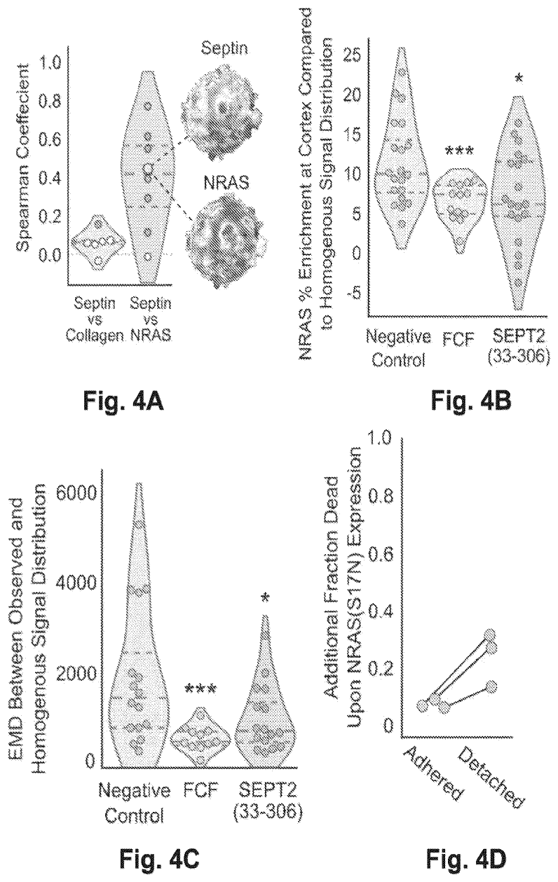

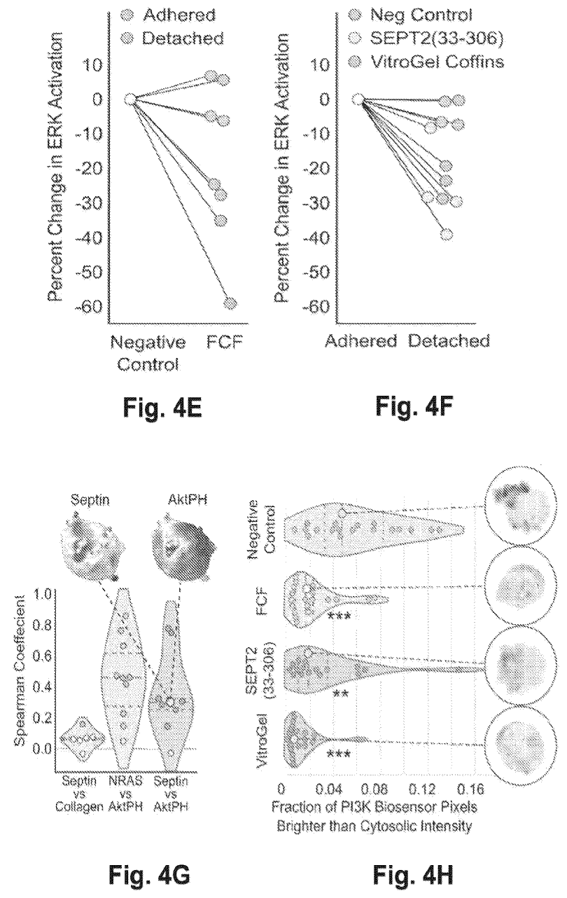

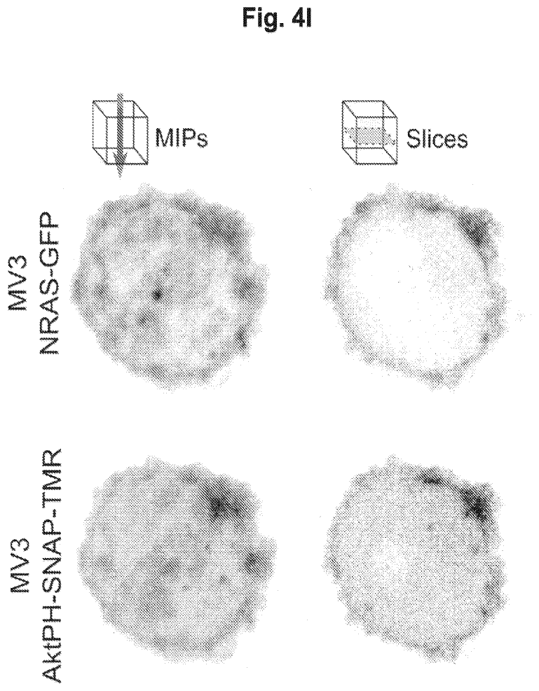

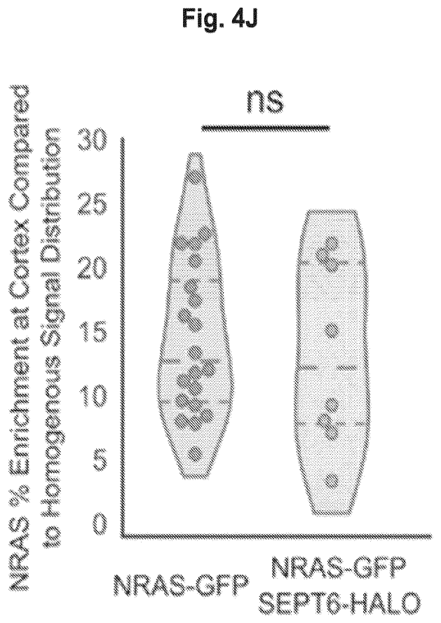

To support these proteomic results observed herein, live cell imaging studies were performed to confirm that the SEPT6/NRAS interaction occurred in rounded, detached cells. Ectopically expressed NRAS-GFP co-localized with cortical septins in bright patches (FIG. 4A). Moreover, perturbation of septin structures using FCF or the dominant negative SEPT2 (33-306) mutant (see FIG. 12A) significantly reduced enrichment of NRAS near the cell surface (FIG. 4B, FIG. 4J).

To further investigate how septin inhibition affected NRAS localization, Optimal Transport analyses, which were borrowed from computer graphics to calculate the Earth Mover's Distance (EMD), were employed as a metric for the spatial heterogeneity of the NRAS signal at the cell surface compared to homogenous distributions of the same amount of signal on the same surface. The results showed that septin-inhibited cells produced lower EMD values (suggesting diffuse signal distributions) than unperturbed cells (FIG. 4C). Taken together, these data show that cortical septin structures bind and scaffold NRAS, affecting its spatial distribution near the surface and increasing its cortical concentration.

### Example 6. Septins Promoted NRAS/ERK and NRAS/PI3K Survival Signaling

To determine whether NRAS signaling is important for anoikis resistance in MV3 cells, NRAS (S17N), a dominant negative mutant known to disrupt NRAS signaling, was overexpressed in adhered and detached MV3 cells. Like bleb and septin inhibition, perturbation of NRAS had little effect on the survival of adhered cells, but consistently increased death in detached cells (FIG. 4D; cell counts for all replicates are provided in Table 5).

TABLE 5

| | MV3 | MV3 |
|---|---|---|
| | Detached | Adhered |
| Experimental 1 | 279 | 256 |
| Control 1 | 453 | 253 |
| Experimental 2 | 388 | 281 |
| Control 2 | 349 | 246 |
| Experimental 3 | 438 | 262 |
| Control 3 | 237 | 235 |

To determine whether septin organization of NRAS and its effectors affected downstream MAPK signaling, the ERK-nKTR nuclear translocation biosensor was used which allowed for monitoring the signaling states of individual cells. Because the prevention of cell-cell adhesion required maintaining ultra-low cell densities, it was opted to use quantitative live cell microscopy rather than Western blotting. After 3 hours of septin inhibition with FCF, adhered cells had no difference in ERK signaling compared to controls, while ERK activation was significantly reduced in detached cells (FIG. 4E; individual counts for all replicates are provided in Table 6).

TABLE 6

| MV3 | Control | FCF |
|---|---|---|
| Adhered | 65 | 61 |
| Adhered | 48 | 59 |
| Adhered | 58 | 53 |
| Adhered | 63 | 63 |
| Detached | 236 | 164 |
| Detached | 212 | 149 |
| Detached | 350 | 258 |
| Detached | 252 | 278 |

Similarly, the effect of 3 hours of detachment vs. adhesion on unperturbed MV3 cells, MV3 cells expressing the dominant negative SEPT2 (33-306) mutant, and MV3 cells grown in Vitrogel coffins was measured. Detachment of uninhibited cells caused little change in ERK activation, while septin and bleb inhibition in detached cells drastically decreased ERK activation (FIG. 4F; individual counts for all replicates are provided in Table 7). Thus, both septins and blebs were necessary for maintaining ERK signaling in detached, but not adhered, MV3 melanoma cells.

TABLE 7

| | MV3 | MV3 |
|---|---|---|
| | Adhered | Detached |
| Control | 65 | 98 |
| Control | 67 | 73 |
| Control | 75 | 73 |
| Control | 69 | 50 |
| SEPT2(33-306) | 63 | 36 |
| SEPT2(33-306) | 33 | 46 |
| SEPT2(33-306) | 42 | 40 |
| SEPT2(33-306) | 69 | 42 |
| VitroGel | 61 | 84 |
| VitroGel | 56 | 64 |
| VitroGel | 55 | 63 |

Because PI3K signaling can be driven by NRAS, and bleb inhibition alters PI3K activity, it was also tested whether septin-mediated cortical scaffolding of NRAS (Q61R) could drive PI3K signaling in detached MV3 cells-thereby activating a second survival pathway. Using the PI3K activity biosensor Akt-PH-GFP, high PI3K activity was co-localized with both septins and NRAS in rounded, poorly-adhered MV3 cells embedded in soft collagen, in support of septin-scaffolded NRAS signaling through PI3K (FIG. 4G and FIG. 4I). Septin inhibition was then performed, either through FCF treatment or expression of SEPT2 (33-306), and the resulting effect upon PI3K signaling was measured. In both cases, PI3K activity fell significantly (FIG. 4H). Similarly, bleb inhibition with VitroGel coffins greatly depleted PI3K activity (FIG. 4H). Altogether, these data showed that bleb and septin inhibition specifically reduced both ERK and PI3K activation levels in detached MV3 melanoma cells, supporting the idea that bleb-nucleated cortical septin structures promoted anoikis resistance through the scaffolding and upregulation of NRAS/RAF/MEK/ERK and NRAS/PI3K/AKT signaling.

### Example 7. Prolonging Post-Detachment Blebbing in Non-Cancerous Fibroblasts Confered Bleb- and Septin-Dependent Anoikis Resistance

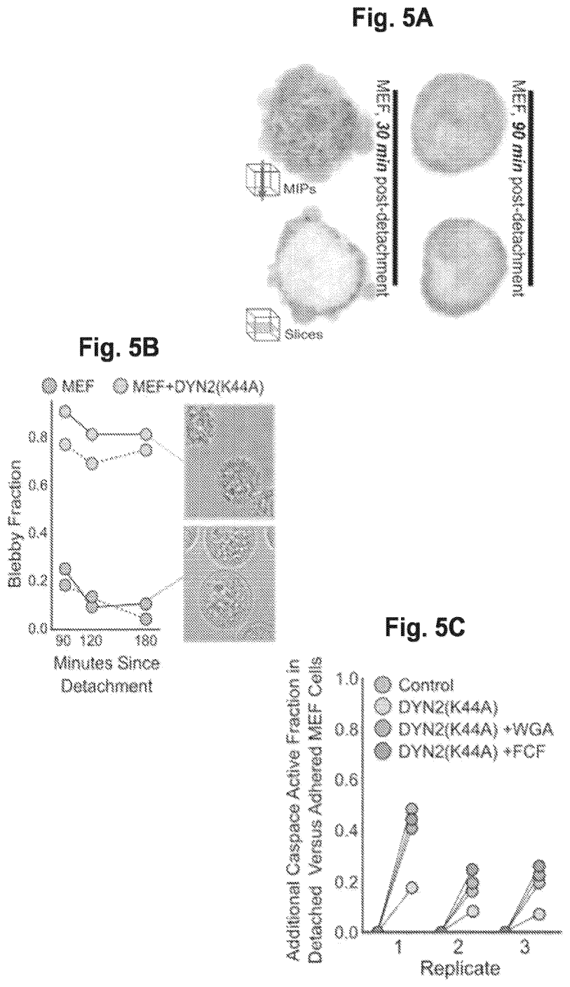

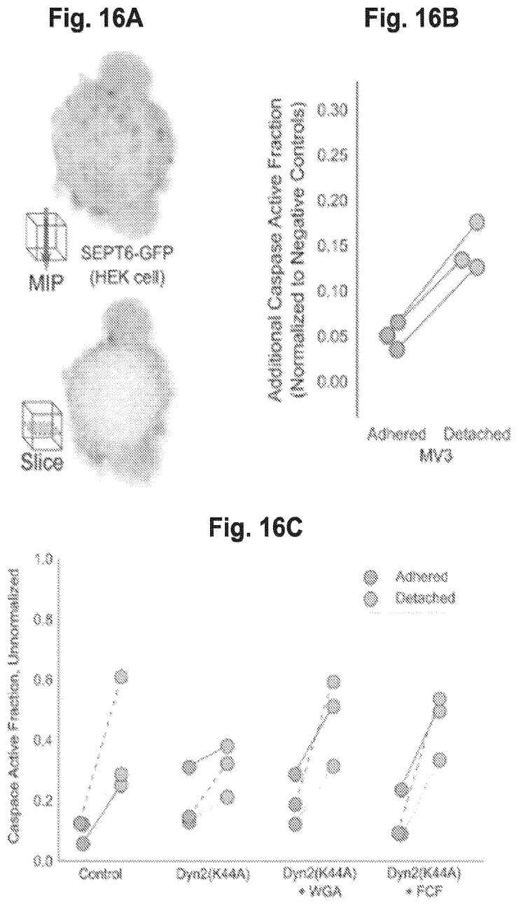

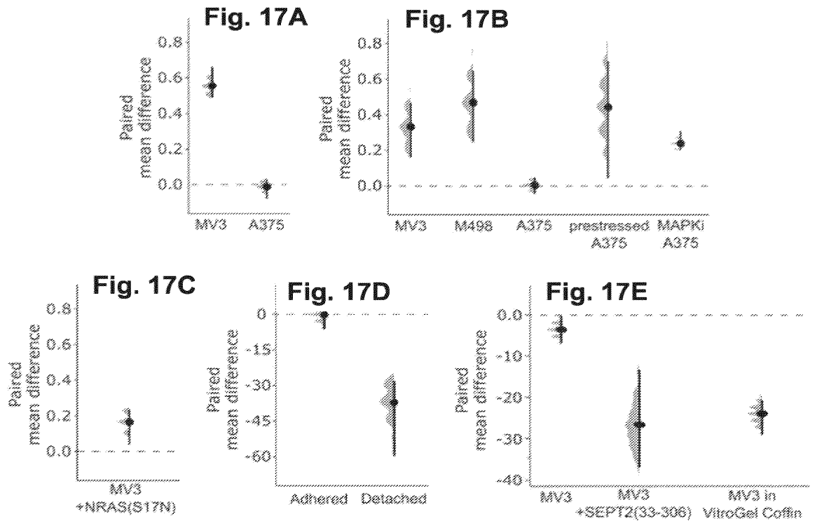

Detached MEF cells expressing SEPT6-GFP were imaged either before or after bleb attenuation. As shown in FIG. 5A and FIG. 16A, cells began blebbing upon detachment from substrate. Specifically, both mouse embryonic fibroblast (MEF) (FIG. 5A) and human epithelial kidney 293 (HEK) cells expressing SEPT6-GFP (FIG. 16A) displayed bright bleb-associated cortical septin structures when imaged before bleb attenuation. While cancer cells, such as melanoma, will continue to bleb indefinitely, most cells will cease blebbing over the course of an hour via an endocytosis-dependent process. To determine if anoikis resistance can be achieved in non-cancerous mouse fibroblasts, MEF cells were allowed to continue past the one-hour mark (FIG. 5B). When blebbing ceased cortical septins were absent (FIG. 5A and FIG. 16A). In agreement with the overnight ethidium homodimer viability assays (FIG. 1B-1F), a consistent and specific increase of caspase activation was observed in detached bleb-inhibited MV3 cells (FIG. 16B, individual counts in Table 8). Endocytosis was inhibited in MEF cells by expressing the dominant negative DYN2 (K44A) mutant. Increasing blebs in this way enhanced anoikis resistance (FIG. 5B, individual counts in Table 9) which was reversed by both bleb (+WGA) and septin (+FCF) inhibition (FIG. 5C, individual counts in Table 10).

TABLE 8

| | Adhered | Detached |
|---|---|---|
| Control 1 | 220 | 544 |
| WGA 1 | 143 | 310 |
| Control 2 | 152 | 215 |
| WGA 2 | 182 | 256 |
| Control 3 | 157 | 267 |
| WGA 3 | 134 | 89 |

TABLE 9

| | 90 min | 120 min | 180 min |
|---|---|---|---|
| MEF | 113 | 67 | 99 |
| MEF + DYN(K44A) | 42 | 21 | 21 |
| MEF | 67 | 23 | 27 |
| MEF + DYN2(K44A) | 73 | 16 | 70 |

TABLE 10

| | Adhered | Detached |
|---|---|---|
| Control | 89 | 49 |
| DYN2(K44A) | 55 | 38 |
| DYN2(K44A) + WGA | 25 | 32 |
| DYN2(K44A) + FCF | 33 | 27 |
| Control | 25 | 23 |
| DYN2(K44A) | 39 | 28 |
| DYN2(K44A) + WGA | 43 | 27 |
| DYN2(K44A) + FCF | 34 | 28 |
| Control | 54 | 92 |
| DYN2(K44A) | 84 | 121 |
| DYN2(K44A) + WGA | 115 | 101 |
| DYN2(K44A) + FCF | 70 | 141 |

Specifically, ˜80% of DYN2 (K44A)-expressing MEFs maintained blebbiness 3 hours post-detachment, while for control MEFs the fraction of blebbing cells fell below 10% over the same time period (FIG. 5B and Table 9). To ensure that the difference in caspase activity was not due to an off-target effect of dynamin inhibition cells were treated with either the bleb inhibitor WGA or the septin inhibitor FCF. Additional caspase activity was measured using the CellEvent biosensor after 4 hours in detached MEF cells expressing DYN2 (K44A) compared to paired adhered cells in the absence and presence of both bleb (+WGA) and septin (+FCF) inhibition (FIG. 5C, Table 10 and FIG. 16C).

To ensure that the difference in caspase activity was not due to an off-target effect of dynamin inhibition, MEF cells were treated with either the bleb inhibitor WGA or the septin inhibitor FCF. In both cases it was found that caspase activation was returned to levels seen in matched non-blebbing control cells, meaning that blebbing DYN2 (K44A)-expressing cells were far more resistant to anoikis and the anoikis resistance acquired by DYN2 (K44A) depends on both bleb formation and septins (FIG. 5C, Table 10 and FIG. 16C). These data show that cancer cells derived from human melanomas and non-malignant fibroblasts derived from mouse embryos all seem to employ the same bleb and septin dependent anoikis resistance strategy.

Together, these experiments suggested that bleb signaling was not a mere peculiarity seen in a subset of melanoma cells, but rather a more ancient signaling phenomenon that, at the least, spans the evolutionary divide between humans and mice. In other words, these results using non-cancerous mouse fibroblasts suggest that the bleb signaling phenomenon goes beyond melanoma, potentially being ubiquitous in mammalian cells.

### Summary of Examples 1-7

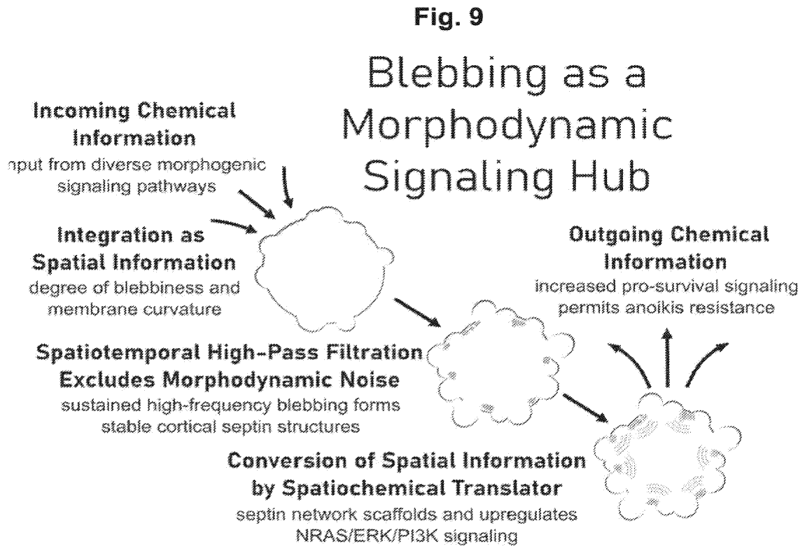

Exemplary methods herein used quantitative subcellular 3D imaging and manipulation of cell morphological states to demonstrate that blebbing triggered the formation of membrane-proximal signaling hubs that initiated signaling cascades leading to anoikis resistance. A schematic of the findings disclosed herein is provided as FIG. 9. Specifically, in melanoma cells blebbing was discovered to generate plasma membrane contours that recruited curvature sensing septin proteins, which acted to scaffold constitutively active mutant NRAS and effectors, driving the upregulation of ERK and PI3K signaling. Inhibition of blebs or septins had little effect on the survival of well-adhered cells, but in detached cells caused NRAS mislocalization, reduced MAPK and PI3K signaling, and ultimately, death. These data provided in the present disclosure unveiled an unanticipated morphological requirement for mutant NRAS to operate as an effective oncoprotein, suggesting novel clinical targets for the treatment of NRAS-driven melanoma. Furthermore, the data defined an unforeseen role for blebs as potent signaling organelles capable of integrating myriad cellular information flows into concerted signaling responses, in this case granting robust anoikis resistance. Data herein revealed that bleb signaling acted as a means of obtaining resistance to targeted therapies for BRAF (V600E) melanoma, and findings herein using non-cancerous mouse fibroblasts suggested the phenomenon goes beyond melanoma, potentially being ubiquitous in mammalian cells.

### Example 8. Melanoma Cells in Mouse Xenografts Recapitulate In Vitro Phenotypes

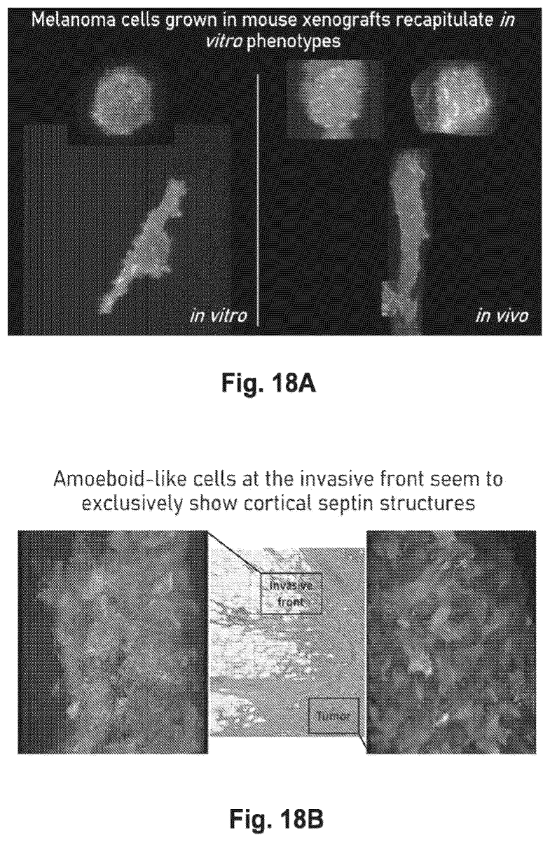

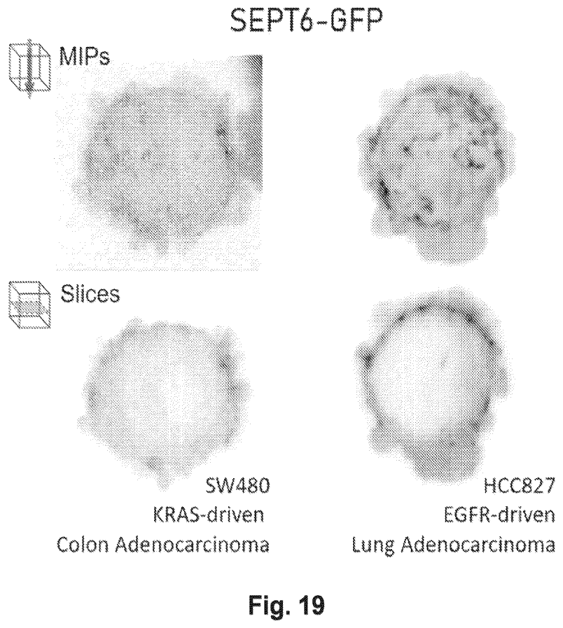

NOD SCID mice were flank-injected with SEPT6-GFP-expressing MV3 melanoma cells to form xenograft tumors. Once tumors were palpable at injection sites they were dissected out with surrounding stroma, fixed, cleared, and imaged via cleared tissue axially-scanned lightsheet microscopy (ctASLM) to observe and quantify bleb formation and septin structure formation. As shown in FIG. 18A and FIG. 18B these in vivo experiments recapitulated in vitro results described previously, showing blebby morphologies with associated punctate septin structures (FIG. 18A). Importantly, this phenotype was exclusively observed at the invasive front of tumors, where cells lose contact to surrounding tissue, and not in the densely packed tumor core (FIG. 18B). These findings agree with previous research showing that blebby amoeboid-like cells are enriched at tumor invasive fronts. The present findings now show that this enrichment of blebbing morphology drives acutely the upregulation survival signals. Furthermore, they support the idea that such bleb-associated septin signaling hubs are relevant in disease progression in human patients.

### Example 9. Bleb-Associated Septin Signaling Hubs are Found in Many Cancer Types Beyond Melanoma

In additional experiments it was found that bleb-associated septin signaling hubs are found in many cancer types beyond melanoma. Specifically, FIG. 19 depicts bleb-associated septin structures in KRAS mutant-driven SW480 colon cancer line and EGFR-driven HCC827 lung adenocarcinoma cancer line (depicted and analyzed using methods described in Examples 1-7 above). As shown, these SW480 and HCC827 colon and lung adenocarcinoma cells show septin localizations very similar to those found in melanoma cells. Notably, in all cell lines found to show such a phenotype (MV3, M498, MAPKi-treated A375, and MEF cells) septin-mediated bleb signaling was also found to be a requirement for attachment-free survival. The one cell line that has no observable bleb-associated septins (basal A375 cells) survives independent of blebs and septins. That bleb-associated septins are found in KRAS-driven colon cancer cells and EGFR-driven lung cancer cells indicates that septin-mediated bleb signaling is important for their survival, and furthermore, that such signaling might be important for a wide variety of cancer types beyond melanoma.

### Example 10. Septin Signaling Hubs can be Pharmaceutically Targeted

In further experiments it was found that in mouse models of melanoma, septin signaling hubs can be pharmaceutically targeted. Specifically, FIG. 20A and FIG. 20B depict graphs illustrating the feasibility of anti-septin therapy in vivo. In triplicate experiments, mice growing tumors formed with SEPT6-GFP expressing MV3 melanoma cells (using the methods described in Example 8 above) were treated with FCF via oral gavage and septin structures were analyzed. The invasive fronts of these tumors were imaged with ctASLM and septin-dense regions at these invasive fronts were digitally subdivided into 50×50×50 μm cubes. Septin foci were segmented using Otsu thresholding, measuring their volume and intensity. It was found that septin structures at the invasive front were disrupted after FCF treatment, with invading amoeboid cells producing significantly fewer (FIG. 20A) and dimmer (FIG. 20B) septin foci. This demonstrates that septin hubs are vulnerable to pharmaceutical therapy in living mammals.

### Example 11. Bleb Associated Septin Signaling Hubs are Present in Human Clinical Samples

In additional experiments it was found that bleb-associated septin signaling hubs are present in human clinical samples. Specifically, FIG. 21 depicts representative images showing bleb-associated septin signaling hubs in cells found in a peritoneal effusion biopsy of a patient with a HER2-amplified mucosal melanoma immunolabeled with an antibody against SEPT2 (Sigma-Aldrich antibody HPA018481). This demonstrates that the phenotype of rounded, blebby cells possessing bleb-associated septin signaling hubs observed in experiments both in vitro and in vivo in the previous examples are also found in clinical samples, further supporting a role in human cancers.

### Methods Used in Examples 1-11

Provided herein are exemplary methods and exemplary materials used in the examples of the present disclosure detailed as follows:

Cell Culture and Reagents. MV3 cells were obtained from MD Anderson Cancer Center. A375 cells (ATCCR CRL1619) were acquired from ATCC. M498 cells were acquired from UT Southwestern. MV3, A375, and M498 cells were cultured in DMEM (Gibco) supplemented with 10% fetal bovine serum (FBS; ThermoFisher) at 37° C. and 5% CO2. Targeted deep sequencing of 1385 cancer-related genes show sequence and copy number variations in MV3, A375, and M498. “Pre-stressed A375” cells were generated by allowing A375 cells to grow to confluency and then grown for an additional 48 hours without passaging, with media changed daily. “MAPKi A375” cells were generated by treating A375 cells with a combination of 10 nM Dabrafenib and 1 nm Trametinib (alternate concentrations used where noted in the text) for 48 hours.

Inhibitors. Wheat Germ Agglutinin (WGA) was purchased from Sigma (product #L9640). Forchlorfenuron (FCF) was purchased from Sigma (product #C2791). VitroGel and VitroGel-RGD were purchased from TheWell Bioscience (sku #s TWG001 and TWG002). H1152 was purchased from Tocris (catalogue #2414). NSC6683394 was purchased from Sigma (product #341216). Dabrafenib (GSK2118436), and Trametinib (GSK1120212) were purchased from Selleckchem. Transient expression of dominant negative Dyn2 (K44A) 66,67 was achieved through adenovirus transduction68 as previously described69 with cells analyzed 16-18 hours after transduction.

Recombinant DNA Constructs. Mouse SEPT6-GFP construct was purchased from Addgene (Addgene plasmid #38296) and was cloned into the pLVX-IRES-puro vector (Clontech). The GFP-AktPH construct as described in Haugh et al., J Cell Biol. 2000; 151 (6): 1269-1280, the disclosure of which is incorporated herein in its entirety, was cloned into the pLVX-IRES-puro vector (Clontech). The GFP-tractin construct (Addgene plasmid #58473) and was cloned into the pLVX-IRES-puro vector (Clontech). The BioID2 construct was obtained from Addgene (Addgene plasmid #74223) and cloned onto the N-terminus of SEPT6 from SEPT6-GFP-pLVX-IRES-puro, replacing eGFP but maintaining the same 22 amino acid linker. SEPT6-HALO was made by cloning the HALO tag from pFN21K (Promega cat #G2821) onto the N-terminus of SEPT6 from SEPT6-GFP-pLVX-IRES-puro, replacing eGFP but maintaining the same 22 amino acid linker. ERK-nKTR-GFP was purchased from Addgene (Addgene plasmid #59150). C-terminally mGFP-tagged human NRAS in pLenti-C-mGFP was purchased from OriGene (OriGene cat #RC202681L2). C-terminal mGFP was removed and eGFP tag was cloned onto the N-terminus after aberrant localization was observed upon expression in MV3 cells (presumably due to steric inhibition of C-terminal palmitoylation and farnesylation domains by the C-terminal mGFP tag). NRAS (S17N) mutant was generated by cloning the S17N mutation into untagged NRAS-pLenti construct using HiFi assembly. pBOB-Septin2-GFP was purchased from Addgene (Addgene plasmid #118734), aa 1-32 and 307-361 were removed via PCR to create SEPT2 (33-306), and the construct was cloned into pLVX-IRES-puro vector (Clontech). SEPT6 (AAH)-GFP was generated by removing aa 355-372 of SEPT6 from SEPT6-GFP-pLVX-IRES-puro via PCR. H2B-mCherry was obtained from Addgene (Addgene #89766). SEPT2 (33-306)-pLVX-IRES-puro (SEQ ID NO: 4, Table below) was generated by purchasing pBOB-Septin2-GFP from Addgene (Addgene plasmid #118734) and removing aa 1-32 and 307-361 via PCR using primers (e.g., SEPT2 (33-306) Fwd Primer (SEQ ID NO: 1) and SEPT2 (33-306) Rev Primer (SEQ ID NO: 2)) as provided in Table below to create SEPT2 (33-306). The construct was then cloned into pLVX-IRES-puro vector (Clontech). SEPT6 (AAH)-GFP (SEQ ID NO: 5, Table below) was generated by purchasing a gBlock gene fragment (Integrated DNA Technologies, SEQ ID NO: 3, Table below) comprised of a GFP-tagged mouse SEPT6 coding region with aa 355-372 removed, which was then cloned into the pLVX-IRES-puro vector (Clontech). All cloning was confirmed via DNA sequencing. Cells expressing lentiviral vectors were created by following the manufacturer's instructions for virus preparation and cell infection (Clontech). Cells were selected for expression by treatment with puromycin and by using fluorescence activated cell sorting. Cells expressing lentiviral vectors were created by following the manufacturer's instructions for virus preparation and cell infection (Clontech). Cells were selected for expression by treatment with puromycin, G418, or by using fluorescence activated cell sorting.

| Sequence | | SEQ ID |
|---|---|---|
| Description | Sequence | NO: |
| SEPT2(33-306) | ccggtgaattcatgaaaaaaggttttgagttcacactg | 1 |
| Fwd Primer: | | |
| SEPT2(33-306) | ctagtctcgagtcatctcttgagtctctcagaacg | 2 |
| Rev Primer | | |
| SEPT6(ΔAH)-GFP | gctgtacaagtccggactcagatctcgagctcaagcttcgctggctatcagtggaggcggt | 3 |
| gBlock fragment: | gggtctgctctggcaagcggtgggggaggctccgcaggtattctgatggcagcggccgat | |
| | atagctcgccaggtgggcgaagattgtcgaactgtccccctggctggacatgtggggttcg | |
| | acagcttgcctgaccagctggtgaataagtcagtcagccagggcttctgcttcaacatcctg | |
| | tgcgtgggggagacaggtttgggcaagtccacgctcatggataccctgtttaacaccaagt | |
| | ttgaaggggagccagcgacccacacacagccaggggttcagctccagtccaataccta | |
| | cgacctccaggagagcaacgtggggttgaaactcacaattgtgagcactgtaggctttgg | |
| | ggatcagatcaacaaggaggacagctacaagcccatcgtggaattcattgatgctcagttt | |
| | gaggcctacctacaagaggagctgaagatccgaagagtactgcactcctaccatgactc | |
| | ccgaatccatgtctgcttgtatttcattgccccgacaggacattcgcttaagtctctggaccta | |
| | gtgaccatgaagaagctggatagtaaggtgaacatcattcccgttattgccaagtcagatg | |
| | ccatttctaagagtgagctggcaaagttcaagatcaaaatcaccagcgaacttgtgagca | |
| | atggagtccagatctatcagttccccacagacgatgagtcggcccacctgccgtttgctgtt | |
| | gttggcagcacagaagaagtgaagataggcaacaagatgatgcgggctcggcagtatc | |
| | cttgggggactgtgcaggttgaaaacgaggctcactgcgactttgtgaagctgcgggaga | |
| | tgctgattcgggtcaacatggaggatctgcgggagcagacccacgcccggcactatgaa | |
| | ttgtaccgccgctgtaagctggaggagatgggcttcaaggacaccgaccctgacagcaa | |
| | accattcagtttgcaggagacatatgaggcaaagagaaatgagttccttggggagctcca | |
| | gaagaaagaagaggagatgaggcagatgtttgtccagagagtcaaagagaaagaag | |
| | cgctgcaccaggaagagaagaagaagctggaggataagaagaaatgtctggatgagg | |
| | aaatgaacgccttcaagcagagaaaggcggcagctgagctgctccagtctcagggctct | |
| | caggctgggggctcacagactctgaaaagggacaaagagaagaagaactttttttaagg | |
| | atcccgcccctctccctccccc | |
| SEPT2(33-306)- | tggaagggctaattcactcccaaagaagacaagatatccttgatctgtggatctaccacac | 4 |
| pLVX-IRES-puro: | acaaggctacttccctgattagcagaactacacaccagggccaggggtcagatatccact | |
| | gacctttggatggtgctacaagctagtaccagttgagccagataaggtagaagaggcca | |
| | ataaaggagagaacaccagcttgttacaccctgtgagcctgcatgggatggatgacccg | |
| | gagagagaagtgttagagtggaggtttgacagccgcctagcatttcatcacgtggcccga | |
| | gagctgcatccggagtacttcaagaactgctgatatcgagcttgctacaagggactttccg | |
| | ctggggactttccagggaggcgtggcctggggggactggggagtggcgagccctcag | |
| | atcctgcatataagcagctgctttttgcctgtactgggtctctctggttagaccagatctgagcc | |
| | tgggagctctctggctaactagggaacccactgcttaagcctcaataaagcttgccttgagt | |
| | gcttcaagtagtgtgtgcccgtctgttgtgtgactctggtaactagagatccctcagaccctttt | |
| | agtcagtgtggaaaatctctagcagtggcgcccgaacagggacttgaaagcgaaaggg | |
| | aaaccagaggagctctctcgacgcaggactcggcttgctgaagcgcgcacggcaagag | |
| | gcgaggggcggcgactggtgagtacgccaaaaattttgactagcggaggctagaagga | |
| | gagagatgggtgcgagagcgtcagtattaagcgggggagaattagatcgcgatgggaa | |
| | aaaattcggttaaggccagggggaaagaaaaaatataaattaaaacatatagtatgggc | |
| | aagcagggagctagaacgattcgcagttaatcctggcctgttagaaacatcagaaggctg | |
| | tagacaaatactgggacagctacaaccatcccttcagacaggatcagaagaacttagat | |
| | cattatataatacagtagcaaccctctattgtgtgcatcaaaggatagagataaaagacac | |
| | caaggaagctttagacaagatagaggaagagcaaaaaaaagtaagaccaccgcac | |
| | agcaagcggccggccgctgatcttcagacctggaggaggagatatgagggacaattgg | |
| | agaagtgaattatataaatataaagtagtaaaaattgaaccattaggagtagcacccacc | |
| | aaggcaaagagaagagtggtgcagagagaaaaaagagcagtgggaataggagcttt | |
| | gttccttgggttcttgggagcagcaggaagcactatgggcgcagcgtcaatgacgctgac | |
| | ggtacaggccagacaattattgtctggtatagtgcagcagcagaacaatttgctgagggct | |
| | attgaggcgcaacagcatctgttgcaactcacagtctggggcatcaagcagctccaggca | |
| | agaatcctggctgtggaaagatacctaaaggatcaacagctcctggggatttggggttgct | |
| | ctggaaaactcatttgcaccactgctgtgccttggaatgctagttggagtaataaatctctgg | |
| | aacagatttggaatcacacgacctggatggagtgggacagagaaattaacaattacaca | |
| | agcttaatacactccttaattgaagaatcgcaaaaccagcaagaaaagaatgaacaag | |
| | aattattggaattagataaatgggcaagtttgtggaattggtttaacataacaaattggctgtg | |
| | gtatataaaattattcataatgatagtaggaggcttggtaggtttaagaatagtttttgctgtact | |
| | ttctatagtgaatagagttaggcagggatattcaccattatcgtttcagacccacctcccaac | |
| | cccgaggggacccgacaggcccgaaggaatagaagaagaaggtggagagagaga | |
| | cagagacagatccattcgattagtgaacggatctcgacggtatcgcctttaaaagaaaag | |
| | gggggattggggggtacagtgcaggggaaagaatagtagacataatagcaacagaca | |
| | tacaaactaaagaattacaaaaacaaattacaaaaattcaaaattttcgggtttattacagg | |
| | gacagcagagatccagtttatcgataagcttgggagttccgcgttacataacttacggtaaa | |
| | tggcccgcctggctgaccgcccaacgacccccgcccattgacgtcaataatgacgtatgtt | |
| | cccatagtaacgccaatagggactttccattgacgtcaatgggtggagtatttacggtaaac | |
| | tgcccacttggcagtacatcaagtgtatcatatgccaagtacgccccctattgacgtcaatg | |
| | acggtaaatggcccgcctggcattatgcccagtacatgaccttatgggactttcctacttggc | |
| | agtacatctacgtattagtcatcgctattaccatggtgatgcggttttggcagtacatcaatgg | |
| | gcgtggatagcggtttgactcacggggatttccaagtctccaccccattgacgtcaatggg | |
| | agtttgttttggcaccaaaatcaacgggactttccaaaatgtcgtaacaactccgccccattg | |
| | acgcaaatgggcggtaggcgtgtacggtgggaggtctatataagcagagctcgtttagtg | |
| | aaccgtcagatcgcctggagacgccatccacgctgttttgacctccatagaagacaccga | |
| | ctctactagaggatctatttccggtgaattcgccaccaatgaaaaaggttttgagttcacact | |
| | gatggtggtcggtgaatcaggtctaggaaaatcgactctcataaacagcctattcctaactg | |
| | atctgtacccagaaagagtcatacctggagcagcagaaaaaattgaaagaactgtccag | |
| | attgaggcttcaactgttgaaattgaagagcgaggggtcaagctacgcctgacagtggta | |
| | gatacccctggctatggtgacgctatcaactgcagagattgttttaagacaattatctcctata | |
| | ttgatgagcaatttgagaggtacctgcatgacgagagcggcttgaacaggcggcacatca | |
| | ttgataatagggtgcattgttgcttttactttatttcaccttttggacatggacttaagcccttagat | |
| | gtggcgtttatgaaggcaatacacaacaaggtgaatattgtgcctgtcattgcaaaagctg | |
| | acactctcaccctgaaggaacgggagcggctgaagaaaaggattctggatgaaattga | |
| | agaacataacatcaaaatctatcacttacctgatgcagaatcagatgaagatgaagatttt | |
| | aaagagcagactagacttctcaaggctagcatcccattctctgtggttggatccaatcagtt | |
| | gattgaagccaaaggaaagaaggtcagaggccgcctctacccctggggtgttgtggaag | |
| | tggagaacccagagcacaatgactttctgaagctgagaaccatgctcatcacccacatgc | |
| | aggatctccaggaggtgacccaggaccttcattatgaaaacttccgttctgagagactcaa | |
| | gagataaactagttctagagcggccgcggatcccgcccctctccctcccccccccctaac | |
| | gttactggccgaagccgcttggaataaggccggtgtgcgtttgtctatatgttattttccaccat | |
| | attgccgtcttttggcaatgtgagggcccggaaacctggccctgtcttcttgacgagcattcct | |
| | aggggtctttcccctctcgccaaaggaatgcaaggtctgttgaatgtcgtgaaggaagcag | |
| | ttcctctggaagcttcttgaagacaaacaacgtctgtagcgaccctttgcaggcagcggaa | |
| | ccccccacctggcgacaggtgcctctgcggccaaaagccacgtgtataagatacacctg | |
| | caaaggcggcacaaccccagtgccacgttgtgagttggatagttgtggaaagagtcaaat | |
| | ggctctcctcaagcgtattcaacaaggggctgaaggatgcccagaaggtaccccattgta | |
| | tgggatctgatctggggcctcggtgcacatgctttacatgtgtttagtcgaggttaaaaaaac | |
| | gtctaggccccccgaaccacggggacgtggttttcctttgaaaaacacgatgataagcttg | |
| | ccacaaccatggctgaacaagatggattgcacgcaggttctccggccgcttgggtggag | |
| | aggctattcggctatgactgggcacaacagacaatcggctgctctgatgccgccgtgttcc | |
| | ggctgtcagcgcaggggcgcccggttctttttgtcaagaccgacctgtccggtgccctgaat | |
| | gaactgcaggacgaggcagcgcggctatcgtggctggccacgacgggcgttccttgcgc | |
| | agctgtgctcgacgttgtcactgaagcgggaagggactggctgctattgggcgaagtgcc | |
| | ggggcaggatctcctgtcatctcaccttgctcctgccgagaaagtatccatcatggctgatg | |
| | caatgcggcggctgcatacgcttgatccggctacctgcccattcgaccaccaagcgaaac | |
| | atcgcatcgagcgagcacgtactcggatggaagccggtcttgtcgatcaggatgatctgg | |
| | acgaagagcatcaggggctcgcgccagccgaactgttcgccaggctcaaggcgcgcat | |
| | gcccgacggcgaggatctcgtcgtgacccatggcgatgcctgcttgccgaatatcatggtg | |
| | gaaaatggccgcttttctggattcatcgactgtggccggctgggtgtggcggaccgctatca | |
| | ggacatagcgttggctacccgtgatattgctgaagagcttggcggcgaatgggctgaccg | |
| | cttcctcgtgctttacggtatcgccgctcccgattcgcagcgcatcgccttctatcgccttcttg | |
| | acgagttcttctgaacgcgtctggaacaatcaacctctggattacaaaatttgtgaaagattg | |
| | actggtattcttaactatgttgctccttttacgctatgtggatacgctgctttaatgcctttgtatcat | |
| | gctattgcttcccgtatggctttcattttctcctccttgtataaatcctggttgctgtctctttatgagg | |
| | agttgtggcccgttgtcaggcaacgtggcgtggtgtgcactgtgtttgctgacgcaaccccc | |
| | actggttggggcattgccaccacctgtcagctcctttccgggactttcgctttccccctccctat | |
| | tgccacggcggaactcatcgccgcctgccttgcccgctgctggacaggggctcggctgttg | |
| | ggcactgacaattccgtggtgttgtcggggaagctgacgtcctttccatggctgctcgcctgt | |
| | gttgccacctggattctgcgcgggacgtccttctgctacgtcccttcggccctcaatccagcg | |
| | gaccttccttcccgcggcctgctgccggctctgcggcctcttccgcgtcttcgccttcgccctc | |
| | agacgagtcggatctccctttgggccgcctccccgcctggaattaattctgcagtcgagac | |
| | ctagaaaaacatggagcaatcacaagtagcaatacagcagctaccaatgctgattgtgc | |
| | ctggctagaagcacaagaggaggaggaggtgggttttccagtcacacctcaggtaccttt | |
| | aagaccaatgacttacaaggcagctgtagatcttagccactttttaaaagaaaagagggg | |
| | actggaagggctaattcactcccaacgaagacaagatatccttgatctgtggatctaccac | |
| | acacaaggctacttccctgattagcagaactacacaccagggccaggggtcagatatcc | |
| | actgacctttggatggtgctacaagctagtaccagttgagccagataaggtagaagaggc | |
| | caataaaggagagaacaccagcttgttacaccctgtgagcctgcatgggatggatgacc | |
| | cggagagagaagtgttagagtggaggtttgacagccgcctagcatttcatcacgtggccc | |
| | gagagctgcatccggagtacttcaagaactgctgatatcgagcttgctacaagggactttc | |
| | cgctggggactttccagggaggcgtggcctggggggactggggagtggcgagccctc | |
| | agatcctgcatataagcagctgctttttgcctgtactgggtctctctggttagaccagatctga | |
| | gcctgggagctctctggctaactagggaacccactgcttaagcctcaataaagcttgccttg | |
| | agtgcttcaagtagtgtgtgcccgtctgttgtgtgactctggtaactagagatccctcagacc | |
| | cttttagtcagtgtggaaaatctctagcagtagtagttcatgtcatcttattattcagtatttataa | |
| | cttgcaaagaaatgaatatcagagagtgagaggccttgacattgctagcgtttaccgtcga | |
| | cctctagctagagcttggcgtaatcatggtcatagctgtttcctgtgtgaaattgttatccgctc | |
| | acaattccacacaacatacgagccggaagcataaagtgtaaagcctggggtgcctaatg | |
| | agtgagctaactcacattaattgcgttgcgctcactgcccgctttccagtcgggaaacctgtc | |
| | gtgccagctgcattaatgaatcggccaacgcgcggggagaggcggtttgcgtattgggcg | |
| | ctcttccgcttcctcgctcactgactcgctgcgctcggtcgttcggctgcggcgagcggtatc | |
| | agctcactcaaaggcggtaatacggttatccacagaatcaggggataacgcaggaaag | |
| | aacatgtgagcaaaaggccagcaaaaggccaggaaccgtaaaaaggccgcgttgctg | |
| | gcgtttttccataggctccgcccccctgacgagcatcacaaaaatcgacgctcaagtcag | |
| | aggtggcgaaacccgacaggactataaagataccaggcgtttccccctggaagctccct | |
| | cgtgcgctctcctgttccgaccctgccgcttaccggatacctgtccgcctttctcccttcggga | |
| | agcgtggcgctttctcatagctcacgctgtaggtatctcagttcggtgtaggtcgttcgctcca | |
| | agctgggctgtgtgcacgaaccccccgttcagcccgaccgctgcgccttatccggtaacta | |
| | tcgtcttgagtccaacccggtaagacacgacttatcgccactggcagcagccactggtaa | |
| | caggattagcagagcgaggtatgtaggcggtgctacagagttcttgaagtggtggcctaa | |
| | ctacggctacactagaagaacagtatttggtatctgcgctctgctgaagccagttaccttcg | |
| | gaaaaagagttggtagctcttgatccggcaaacaaaccaccgctggtagcggtggttttttt | |
| | gtttgcaagcagcagattacgcgcagaaaaaaaggatctcaagaagatcctttgatcttttc | |
| | tacggggtctgacgctcagtggaacgaaaactcacgttaagggattttggtcatgagattat | |
| | caaaaaggatcttcacctagatccttttaaattaaaaatgaagttttaaatcaatctaaagtat | |
| | atatgagtaaacttggtctgacagttaccaatgcttaatcagtgaggcacctatctcagcgat | |
| | ctgtctatttcgttcatccatagttgcctgactccccgtcgtgtagataactacgatacgggag | |
| | ggcttaccatctggccccagtgctgcaatgataccgcgagacccacgctcaccggctcca | |
| | gatttatcagcaataaaccagccagccggaagggccgagcgcagaagtggtcctgcaa | |
| | ctttatccgcctccatccagtctattaattgttgccgggaagctagagtaagtagttcgccagt | |
| | taatagtttgcgcaacgttgttgccattgctacaggcatcgtggtgtcacgctcgtcgtttggta | |
| | tggcttcattcagctccggttcccaacgatcaaggcgagttacatgatcccccatgttgtgca | |
| | aaaaagcggttagctccttcggtcctccgatcgttgtcagaagtaagttggccgcagtgttat | |
| | cactcatggttatggcagcactgcataattctcttactgtcatgccatccgtaagatgcttttctg | |
| | tgactggtgagtactcaaccaagtcattctgagaatagtgtatgcggcgaccgagttgctctt | |
| | gcccggcgtcaatacgggataataccgcgccacatagcagaactttaaaagtgctcatca | |
| | ttggaaaacgttcttcggggcgaaaactctcaaggatcttaccgctgttgagatccagttcg | |
| | atgtaacccactcgtgcacccaactgatcttcagcatcttttactttcaccagcgtttctgggtg | |
| | agcaaaaacaggaaggcaaaatgccgcaaaaaagggaataagggcgacacggaa | |
| | atgttgaatactcatactcttcctttttcaatattattgaagcatttatcagggttattgtctcatga | |
| | gcggatacatatttgaatgtatttagaaaaataaacaaataggggttccgcgcacatttccc | |
| | cgaaaagtgccacctgacgtcgacggatcgggagatcaacttgtttattgcagcttataatg | |
| | gttacaaataaagcaatagcatcacaaatttcacaaataaagcatttttttcactgcattctag | |
| | ttgtggtttgtccaaactcatcaatgtatcttatcatgtctggatcaactggataactcaagcta | |
| | accaaaatcatcccaaacttcccaccccataccctattaccactgccaattacctgtggtttc | |
| | atttactctaaacctgtgattcctctgaattattttcattttaaagaaattgtatttgttaaatatgta | |
| | ctacaaacttagtagt | |
| SEPT6(ΔAH)- | tggaagggctaattcactcccaaagaagacaagatatccttgatctgtggatctaccacac | 5 |
| GFP-pLVX-IRES- | acaaggctacttccctgattagcagaactacacaccagggccaggggtcagatatccact | |
| puro: | gacctttggatggtgctacaagctagtaccagttgagccagataaggtagaagaggcca | |
| | ataaaggagagaacaccagcttgttacaccctgtgagcctgcatgggatggatgacccg | |
| | gagagagaagtgttagagtggaggtttgacagccgcctagcatttcatcacgtggcccga | |
| | gagctgcatccggagtacttcaagaactgctgatatcgagcttgctacaagggactttccg | |
| | ctggggactttccagggaggcgtggcctgggcgggactggggagtggcgagccctcag | |
| | atcctgcatataagcagctgctttttgcctgtactgggtctctctggttagaccagatctgagcc | |
| | tgggagctctctggctaactagggaacccactgcttaagcctcaataaagcttgccttgagt | |
| | gcttcaagtagtgtgtgcccgtctgttgtgtgactctggtaactagagatccctcagaccctttt | |
| | agtcagtgtggaaaatctctagcagtggcgcccgaacagggacttgaaagcgaaaggg | |
| | aaaccagaggagctctctcgacgcaggactcggcttgctgaagcgcgcacggcaagag | |
| | gcgaggggcggcgactggtgagtacgccaaaaattttgactagcggaggctagaagga | |
| | gagagatgggtgcgagagcgtcagtattaagcgggggagaattagatcgcgatgggaa | |
| | aaaattcggttaaggccagggggaaagaaaaaatataaattaaaacatatagtatgggc | |
| | aagcagggagctagaacgattcgcagttaatcctggcctgttagaaacatcagaaggctg | |
| | tagacaaatactgggacagctacaaccatcccttcagacaggatcagaagaacttagat | |
| | cattatataatacagtagcaaccctctattgtgtgcatcaaaggatagagataaaagacac | |
| | caaggaagctttagacaagatagaggaagagcaaaacaaaagtaagaccaccgcac | |
| | agcaagcggccggccgctgatcttcagacctggaggaggagatatgagggacaattgg | |
| | agaagtgaattatataaatataaagtagtaaaaattgaaccattaggagtagcacccacc | |
| | aaggcaaagagaagagtggtgcagagagaaaaaagagcagtgggaataggagcttt | |
| | gttccttgggttcttgggagcagcaggaagcactatgggcgcagcgtcaatgacgctgac | |
| | ggtacaggccagacaattattgtctggtatagtgcagcagcagaacaatttgctgagggct | |
| | attgaggcgcaacagcatctgttgcaactcacagtctggggcatcaagcagctccaggca | |
| | agaatcctggctgtggaaagatacctaaaggatcaacagctcctggggatttggggttgct | |
| | ctggaaaactcatttgcaccactgctgtgccttggaatgctagttggagtaataaatctctgg | |
| | aacagatttggaatcacacgacctggatggagtgggacagagaaattaacaattacaca | |
| | agcttaatacactccttaattgaagaatcgcaaaaccagcaagaaaagaatgaacaag | |
| | aattattggaattagataaatgggcaagtttgtggaattggtttaacataacaaattggctgtg | |
| | gtatataaaattattcataatgatagtaggaggcttggtaggtttaagaatagtttttgctgtact | |
| | ttctatagtgaatagagttaggcagggatattcaccattatcgtttcagacccacctcccaac | |
| | cccgaggggacccgacaggcccgaaggaatagaagaagaaggtggagagagaga | |
| | cagagacagatccattcgattagtgaacggatctcgacggtatcgcctttaaaagaaaag | |
| | gggggattggggggtacagtgcaggggaaagaatagtagacataatagcaacagaca | |
| | tacaaactaaagaattacaaaaacaaattacaaaaattcaaaattttcgggtttattacagg | |
| | gacagcagagatccagtttatcgataagcttgggagttccgcgttacataacttacggtaaa | |
| | tggcccgcctggctgaccgcccaacgacccccgcccattgacgtcaataatgacgtatgtt | |
| | cccatagtaacgccaatagggactttccattgacgtcaatgggtggagtatttacggtaaac | |
| | tgcccacttggcagtacatcaagtgtatcatatgccaagtacgccccctattgacgtcaatg | |
| | acggtaaatggcccgcctggcattatgcccagtacatgaccttatgggactttcctacttggc | |
| | agtacatctacgtattagtcatcgctattaccatggtgatgcggttttggcagtacatcaatgg | |
| | gcgtggatagcggtttgactcacggggatttccaagtctccaccccattgacgtcaatggg | |
| | agtttgttttggcaccaaaatcaacgggactttccaaaatgtcgtaacaactccgccccattg | |
| | acgcaaatgggcggtaggcgtgtacggtgggaggtctatataagcagagctggtttagtg | |
| | aaccgtcagatccgctagcgctaccggtcgccaccatggtgagcaagggcgaggagct | |
| | gttcaccggggggtgcccatcctggtcgagctggacggcgacgtaaacggccacaagtt | |
| | cagcgtgtccggcgagggcgagggcgatgccacctacggcaagctgaccctgaagttc | |
| | atctgcaccaccggcaagctgcccgtgccctggcccaccctcgtgaccaccctgacctac | |
| | ggcgtgcagtgcttcagccgctaccccgaccacatgaagcagcacgacttcttcaagtcc | |
| | gccatgcccgaaggctacgtccaggagcgcaccatcttcttcaaggacgacggcaacta | |
| | caagacccgcgccgaggtgaagttcgagggcgacaccctggtgaaccgcatcgagctg | |
| | aagggcatcgacttcaaggaggacggcaacatcctggggcacaagctggagtacaact | |
| | acaacagccacaacgtctatatcatggccgacaagcagaagaacggcatcaaggtga | |
| | acttcaagatccgccacaacatcgaggacggcagcgtgcagctcgccgaccactacca | |
| | gcagaacacccccatcggcgacggccccgtgctgctgcccgacaaccactacctgagc | |
| | acccagtccgccctgagcaaagaccccaacgagaagcgcgatcacatggtcctgctgg | |
| | agttcgtgaccgccgccgggatcactctcggcatggacgagctgtacaagtccggactca | |
| | gatctcgagctcaagcttcgctggctatcagtggaggcggtgggtctgctctggcaagcgg | |
| | tgggggaggctccgcaggtattctgatggcagcggccgatatagctcgccaggtgggcg | |
| | aagattgtcgaactgtccccctggctggacatgtggggttcgacagcttgcctgaccagctg | |
| | gtgaataagtcagtcagccagggcttctgcttcaacatcctgtgcgtgggggagacaggttt | |
| | gggcaagtccacgctcatggataccctgtttaacaccaagtttgaaggggagccagcga | |
| | cccacacacagccaggggttcagctccagtccaatacctacgacctccaggagagcaa | |
| | cgtggggttgaaactcacaattgtgagcactgtaggctttggggatcagatcaacaagga | |
| | ggacagctacaagcccatcgtggaattcattgatgctcagtttgaggcctacctacaagag | |
| | gagctgaagatccgaagagtactgcactcctaccatgactcccgaatccatgtctgcttgta | |
| | tttcattgccccgacaggacattcgcttaagtctctggacctagtgaccatgaagaagctgg | |
| | atagtaaggtgaacatcattcccgttattgccaagtcagatgccatttctaagagtgagctgg | |
| | caaagttcaagatcaaaatcaccagcgaacttgtgagcaatggagtccagatctatcagtt | |
| | ccccacagacgatgagtcggcccacctgccgtttgctgttgttggcagcacagaagaagt | |
| | gaagataggcaacaagatgatgcgggctcggcagtatccttgggggactgtgcaggttg | |
| | aaaacgaggctcactgcgactttgtgaagctgcgggagatgctgattcgggtcaacatgg | |
| | aggatctgcgggagcagacccacgcccggcactatgaattgtaccgccgctgtaagctg | |
| | gaggagatgggcttcaaggacaccgaccctgacagcaaaccattcagtttgcaggaga | |
| | catatgaggcaaagagaaatgagttccttggggagctccagaagaaagaagaggagat | |
| | gaggcagatgtttgtccagagagtcaaagagaaagaagcgctgcaccaggaagagaa | |
| | gaagaagctggaggataagaagaaatgtctggatgaggaaatgaacgccttcaagcag | |
| | agaaaggcggcagctgagctgctccagtctcagggctctcaggctgggggctcacagac | |
| | tctgaaaagggacaaagagaagaagaactttttttaaggatcccgcccctctccctccccc | |
| | ccccctaacgttactggccgaagccgcttggaataaggccggtgtgcgtttgtctatatgtta | |
| | ttttccaccatattgccgtcttttggcaatgtgagggcccggaaacctggccctgtcttcttgac | |
| | gagcattcctaggggtctttcccctctcgccaaaggaatgcaaggtctgttgaatgtcgtga | |
| | aggaagcagttcctctggaagcttcttgaagacaaacaacgtctgtagcgaccctttgcag | |
| | gcagcggaaccccccacctggcgacaggtgcctctgcggccaaaagccacgtgtataa | |
| | gatacacctgcaaaggcggcacaaccccagtgccacgttgtgagttggatagttgtggaa | |
| | agagtcaaatggctctcctcaagcgtattcaacaaggggctgaaggatgcccagaaggt | |
| | accccattgtatgggatctgatctggggcctcggtgcacatgctttacatgtgtttagtcgagg | |
| | ttaaaaaaacgtctaggccccccgaaccacggggacgtggttttcctttgaaaaacacga | |
| | tgataagcttgccacaacccacaaggagacgaccttccatgaccgagtacaagcccac | |
| | ggtgcgcctcgccacccgcgacgacgtcccccgggccgtacgcaccctcgccgccgcg | |
| | ttcgccgactaccccgccacgcgccacaccgtcgacccggaccgccacatcgagcggg | |
| | tcaccgagctgcaagaactcttcctcacgcgcgtcgggctcgacatcggcaaggtgtggg | |
| | tcgcggacgacggcgccgcggtggcggtctggaccacgccggagagcgtcgaagcgg | |
| | gggcggtgttcgccgagatcggcccgcgcatggccgagttgagcggttcccggctggcc | |
| | gcgcagcaacagatggaaggcctcctggcgccgcaccggcccaaggagcccgcgtg | |
| | gttcctggccaccgtcggcgtctcgcccgaccaccagggcaagggtctgggcagcgccg | |
| | tcgtgctccccggagtggaggcggccgagcgcgccggggtgcccgccttcctggagacc | |
| | tccgcgccccgcaacctccccttctacgagcggctcggcttcaccgtcaccgccgacgtc | |
| | gaggtgcccgaaggaccgcgcacctggtgcatgacccgcaagcccggtgcctagacg | |
| | cgtctggaacaatcaacctctggattacaaaatttgtgaaagattgactggtattcttaactat | |
| | gttgctccttttacgctatgtggatacgctgctttaatgcctttgtatcatgctattgcttcccgtat | |
| | ggctttcattttctcctccttgtataaatcctggttgctgtctctttatgaggagttgtggcccgttgt | |
| | caggcaacgtggcgtggtgtgcactgtgtttgctgacgcaacccccactggttggggcatt | |
| | gccaccacctgtcagctcctttccgggactttcgctttccccctccctattgccacggcggaa | |
| | ctcatcgccgcctgccttgcccgctgctggacaggggctcggctgttgggcactgacaattc | |
| | cgtggtgttgtcggggaagctgacgtcctttccatggctgctcgcctgtgttgccacctggatt | |
| | ctgcgcgggacgtccttctgctacgtcccttcggccctcaatccagcggaccttccttcccgc | |
| | ggcctgctgccggctctgcggcctcttccgcgtcttcgccttcgccctcagacgagtcggat | |
| | ctccctttgggccgcctccccgcctggaattaattctgcagtcgagacctagaaaaacatg | |
| | gagcaatcacaagtagcaatacagcagctaccaatgctgattgtgcctggctagaagca | |
| | caagaggaggaggaggtgggttttccagtcacacctcaggtacctttaagaccaatgactt | |
| | acaaggcagctgtagatcttagccactttttaaaagaaaagaggggactggaagggcta | |
| | attcactcccaacgaagacaagatatccttgatctgtggatctaccacacacaaggctactt | |
| | ccctgattagcagaactacacaccagggccaggggtcagatatccactgacctttggatg | |
| | gtgctacaagctagtaccagttgagccagataaggtagaagaggccaataaaggagag | |
| | aacaccagcttgttacaccctgtgagcctgcatgggatggatgacccggagagagaagt | |
| | gttagagtggaggtttgacagccgcctagcatttcatcacgtggcccgagagctgcatccg | |
| | gagtacttcaagaactgctgatatcgagcttgctacaagggactttccgctggggactttcc | |
| | agggaggcgtggcctgggcgggactggggagtggcgagccctcagatcctgcatataa | |
| | gcagctgctttttgcctgtactgggtctctctggttagaccagatctgagcctgggagctctctg | |
| | gctaactagggaacccactgcttaagcctcaataaagcttgccttgagtgcttcaagtagtg | |
| | tgtgcccgtctgttgtgtgactctggtaactagagatccctcagacccttttagtcagtgtgga | |
| | aaatctctagcagtagtagttcatgtcatcttattattcagtatttataacttgcaaagaaatga | |
| | atatcagagagtgagaggccttgacattgctagcgtttaccgtcgacctctagctagagctt | |
| | ggcgtaatcatggtcatagctgtttcctgtgtgaaattgttatccgctcacaattccacacaac | |
| | atacgagccggaagcataaagtgtaaagcctggggtgcctaatgagtgagctaactcac | |
| | attaattgcgttgcgctcactgcccgctttccagtcgggaaacctgtcgtgccagctgcatta | |
| | atgaatcggccaacgcgcggggagaggcggtttgcgtattgggcgctcttccgcttcctcg | |
| | ctcactgactcgctgcgctcggtcgttcggctgcggcgagcggtatcagctcactcaaagg | |
| | cggtaatacggttatccacagaatcaggggataacgcaggaaagaacatgtgagcaaa | |
| | aggccagcaaaaggccaggaaccgtaaaaaggccgcgttgctggcgtttttccataggc | |
| | tccgcccccctgacgagcatcacaaaaatcgacgctcaagtcagaggtggcgaaaccc | |
| | gacaggactataaagataccaggcgtttccccctggaagctccctcgtgcgctctcctgttc | |
| | cgaccctgccgcttaccggatacctgtccgcctttctcccttcgggaagcgtggcgctttctc | |
| | atagctcacgctgtaggtatctcagttcggtgtaggtcgttcgctccaagctgggctgtgtgc | |
| | acgaaccccccgttcagcccgaccgctgcgccttatccggtaactatcgtcttgagtccaa | |
| | cccggtaagacacgacttatcgccactggcagcagccactggtaacaggattagcagag | |
| | cgaggtatgtaggcggtgctacagagttcttgaagtggtggcctaactacggctacactag | |
| | aagaacagtatttggtatctgcgctctgctgaagccagttaccttcggaaaaagagttggta | |
| | gctcttgatccggcaaacaaaccaccgctggtagcggtggtttttttgtttgcaagcagcag | |
| | attacgcgcagaaaaaaaggatctcaagaagatcctttgatcttttctacggggtctgacgc | |
| | tcagtggaacgaaaactcacgttaagggattttggtcatgagattatcaaaaaggatcttca | |
| | cctagatccttttaaattaaaaatgaagttttaaatcaatctaaagtatatatgagtaaacttgg | |
| | tctgacagttaccaatgcttaatcagtgaggcacctatctcagcgatctgtctatttcgttcatc | |
| | catagttgcctgactccccgtcgtgtagataactacgatacgggagggcttaccatctggcc | |
| | ccagtgctgcaatgataccgcgagacccacgctcaccggctccagatttatcagcaataa | |
| | accagccagccggaagggccgagcgcagaagtggtcctgcaactttatccgcctccatc | |
| | cagtctattaattgttgccgggaagctagagtaagtagttcgccagttaatagtttgcgcaac | |
| | gttgttgccattgctacaggcatcgtggtgtcacgctcgtcgtttggtatggcttcattcagctcc | |
| | ggttcccaacgatcaaggcgagttacatgatcccccatgttgtgcaaaaaagcggttagct | |
| | ccttcggtcctccgatcgttgtcagaagtaagttggccgcagtgttatcactcatggttatggc | |
| | agcactgcataattctcttactgtcatgccatccgtaagatgcttttctgtgactggtgagtact | |
| | caaccaagtcattctgagaatagtgtatgcggcgaccgagttgctcttgcccggcgtcaat | |
| | acgggataataccgcgccacatagcagaactttaaaagtgctcatcattggaaaacgttct | |
| | tcggggcgaaaactctcaaggatcttaccgctgttgagatccagttcgatgtaacccactcg | |
| | tgcacccaactgatcttcagcatcttttactttcaccagcgtttctgggtgagcaaaaacagg | |
| | aaggcaaaatgccgcaaaaaagggaataagggcgacacggaaatgttgaatactcat | |
| | actcttcctttttcaatattattgaagcatttatcagggttattgtctcatgagcggatacatatttg | |
| | aatgtatttagaaaaataaacaaataggggttccgcgcacatttccccgaaaagtgccac | |
| | ctgacgtcgacggatcgggagatcaacttgtttattgcagcttataatggttacaaataaag | |
| | caatagcatcacaaatttcacaaataaagcatttttttcactgcattctagttgtggtttgtccaa | |
| | actcatcaatgtatcttatcatgtctggatcaactggataactcaagctaaccaaaatcatcc | |
| | caaacttcccaccccataccctattaccactgccaattacctgtggtttcatttactctaaacct | |
| | gtgattcctctgaattattttcattttaaagaaattgtatttgttaaatatgtactacaaacttagta | |
| | gtttttaaagaaattgtatttgttaaatatgtactacaaacttagtagt | |

Detached/Adhered Cell Culture. Cells were grown to 70-80% confluency, trypsinized for 3 minutes, resuspended at approximately 100 cells/ml (measured by eye using light microscopy) in DMEM (Gibco) supplemented with 10% fetal bovine serum (FBS; ThermoFisher) and detached from one another by repeated pipetting. Drug treatments, solvent-only controls, or CellEvent caspase activity sensor were added to suspensions if appropriate to the experiment, and then immediately transferred to both an uncoated (for detached cells) and ibiTreated (for adhered cells) 8-well ibidi u-slide (ibidi cat #80821 and 80826) at 200 μl/well. Both slides were stored at 37° C. and 5% CO2 for either 24 hours (hrs) for viability assays, 3 hrs for ERK activity assays or 4 hours for caspase activation assays. Uncoated slides (detached) were nutated at 20 rpm during this time. At the end of this period detached cells were examined by microscopy to confirm that cells did not aggregate-if aggregates were found (results from protocol optimization show this was most commonly caused by wells being seeded at too high a cell density), the experiment was discarded and repeated. For experiments using MAPKi A375 cells, Dabrafenib and Trametinib treatment was continued over the course of the experiment.

Cells Embedded in 3D Collagen. Collagen gels were created by mixing bovine collagen I (Advanced Biomatrix 5005 and 5026) with concentrated phosphate buffered saline (PBS) and water for a final concentration of 2 mg/mL collagen. This collagen solution was then brought to pH 7 with 1N NaOH and mixed with cells just prior to incubation at 37° C. to induce collagen polymerization. Cells were suspended using trypsin/EDTA (Gibco), centrifuged to remove media, and then mixed with collagen just prior to incubation at 37° C. to initiate collagen polymerization. To image collagen fibers, a small amount of collagen was conjugated directly to AlexaFluor 568 dye and mixed with the collagen sample just prior to polymerization. Poorly-adhered cells were enriched in samples by visualizing immediately upon collagen polymerization, and visually selected for those displaying rounded, blebby morphologies.

Cells Grown in VitroGel Coffins. VitroGel coffins were prepared by embedding MV3 or A375 cells in VitroGel 3D or RGD at 1:1 dilution according to manufacturer's instructions (TheWell Biosciences, sku #TWG001). Briefly, cells were grown to 70-80% confluency, trypsinized, and diluted to approximately 500 cells/ml in in DMEM (Gibco) supplemented with 10% fetal bovine serum (FBS; ThermoFisher) and detached from one another by repeated pipetting. A 1:1 solution of VitroGel and VitroGel Dilution Solution was made and added to cell suspension at a ratio of 4:1 (VitroGel: cells) using gentle pipetting until well-mixed (with care taken not to form bubbles). Cell/VitroGel solution was transferred to an 8-well uncoated ibidi μ-slide (ibidi cat #80821) at 100 μl/well, spread over the bottom of wells with a pipette tip, and allowed to polymerize at room temperature for 15 minutes. The remainder of the well was then filled with 10% FBS DMEM and slide was stored at 37° C. and 5% CO2 for either 24 hrs for viability assays, 3 hrs for ERK activity assays, or 1 hour for visualization of septins/PI3K activity.

2D Immunofluorescence. WT MV3 or MV3 cells expressing SEPT6-GFP were seeded in MatTek glass bottom coverslip dishes (P35G-1.5-14-C) at 50,000 cells and incubated overnight. The following day the cells were washed 3 times with 1×PBS and fixed with 370C pre-warmed 4% PFA dissolved in cytoskeletal buffer. The cytoskeletal buffer consists 10 mM PIPES, 100 mM NaCl, 300 mM Sucrose, 1 mM EGTA, 1 mM MgCl2, 1 mM DTT and Protease inhibitor cocktail. The cells were incubated with PFA at 370C for 20 mins. The MV3-Sept6-GFP cells were then imaged on the Nikon Eclipse Ti widefield epifluorescence microscope with 100× Plan APO oil immersion lens using NIS-Elements 4.30.02 (Build 1053). For immunofluorescence staining of wild type unlabeled cells, the cells were then permeabilized with 0.5% Triton X-100 for 20 mins and then blocked with 5% BSA in 1×PBS for 1 hour. Following blocking the cells were then treated with rabbit-anti-SEPT2 primary antibody (Sigma, HPA018481) at a 1:1000 dilution overnight at 40C. The cells were then washed with 1×PBS for 10 minutes for a total of three times and then treated with donkey anti-rabbit-488 secondary antibody (Thermo, A-21206) at a final dilution of 1:5000 for 1 hour. The cells were then washed with 1×PBS for 10 minutes for a total of three times and then imaged on the Nikon Eclipse Ti widefield epifluorescence microscope.

3D Immunofluorescence. MV3 cells were harvested from 10 cm dishes and seeded on Corning Ultra-Low Attachment culture dishes (Sigma, CLS3262) for ˜2 hours for the cells to acquire their blebby architecture. The cells were then centrifuged at 1000 rpm for 5 minutes. The pellet was gently washed once by re-suspending in 1×PBS to remove any media and centrifuged. The pellet was then re-suspended and fixed in 100 μL of pre-warmed 370C, 4% PFA dissolved in cytoskeletal buffer for 20 minutes at 370C. The cytoskeletal buffer consists 10 mM PIPES, 100 mM NaCl, 300 mM Sucrose, 1 mM EGTA, 1 mM MgCl2, 1 mM DTT and Protease inhibitor cocktail. The cells were then washed with PBS, and centrifuged. To permeabilize the cells pellet was resuspended in saponin for 20 minutes. The cells were then centrifuged and the pellet was blocked by re-suspending in 5% BSA for one hour with gentle rocking for uniform blocking. The cells suspension was then centrifuged and re-suspended in rabbit anti-SEPT2 primary antibody (Millipore Sigma, HPA018481) at a concentration of 1:1000 overnight at 4° C. The following day the cells were centrifuged, and washed once in 10 mL of PBS. The cells were then centrifuged and re-suspended in donkey anti-rabbit-488 secondary antibody (Thermo, A-21206) at a final dilution of 1:5000 for 1 hour. The cells were then centrifuged and washed twice with 10 mL 1× PBS by centrifugation. The pellet was then re-suspended in AF568-phalloidin (Invitrogen, A12380) for one hour, washed, DAPI for 5 minutes, washed, and embedded in soft bovine collagen as described above

Viability, ERK Activity, and Caspase ActivityCell Counting Assays. Viability, ERK activity, and caspase activity assays were analyzed using live-cell fluorescent and phase-contrast microscopy, performed on a Nikon Ti microscope equipped with an environmental chamber held at 37° C. and 5% CO2 at 20× magnification. For viability assays, cells were stained with ethidium homodimer-1 (Invitrogen cat #E1169) at 4 UM and Hoechst (ThermoFisher cat #H3570) at 10 μg/ml for 15 minutes before imaging. Live and dead cells (as identified by significant cellular ethidium signal) were counted using the Cell Counter ImageJ plugin. For ERK activity assays, live cells carrying ERK-nKTR-GFP and H2B-mCherry were imaged after 3 hours of experimental conditions and high/low ERK activity cells were counted with Cell Counter ImageJ plugin. Cells in which nuclear GFP signals were by eye higher than cytoplasmic signal (“brighter” nucleus visible within “darker” cytoplasm) were labelled ERK low, while cytoplasmic signals equal to or higher than nuclear signal (nucleus indistinguishable from cytoplasm or “darker” nucleus within “brighter” cytoplasm) were labelled ERK high. To control for unconscious bias in making this determination, the identity of all ERK experimental groups were blinded from the analyst. For caspase activity assays, cells were treated with CellEvent caspase-3/7 green detection reagent (ThermoFisher cat #C10423) according to manufacturer's instructions at a final concentration of 8 μM. Cells were treated upon introduction to adhered or attached culture conditions, as described above. After 4 hours, cells were treated with Hoechst (ThermoFisher cat #H3570) at 10 μg/ml for 15 minutes and imaged. Caspase positive and negative cells (as identified by significant cellular CellEvent signal) were counted using the Cell Counter ImageJ plugin.

Proteomics. MV3, MV3 expressing SEPT6-HALO, A375, and A375 expressing SEPT-Halo cells were each plated in 150 mm dishes at approximately 7.5×10⁶ cells per dish and grown to 70-80% confluency. Pulldowns were performed per manufacturer's instructions (Promega, G6504). Cells were washed and harvested in ice cold PBS. Cells were pelleted at 2000 RCF for 10 minutes at 4° C. Pellets were stored at −80° C. for 72 hours. Pellets were then thawed at room temperature, lysed in Mammalian Lysis Buffer supplemented with Protease Inhibitor cocktail (Promega). The lysate was homogenized with a 27G syringe and centrifuged at 14,000 RCF for 5 min at 4° C. The supernatant was diluted in TBS and incubated with pre-equilibrated HaloLink Resin (Promega) at room temperature with rotation for 15 minutes. Resin was then washed 4 times with Resin Wash Buffer (Promega). Complexed proteins were eluted in SDS Elution Buffer (Promega) for 30 minutes at room temperature. Eluted samples and whole cell lysate controls were loaded and run on a 10% Mini-PROTEAN TGX protein gel (Biorad), visualized with AcquaStain (Bulldog Bio), excised, and analyzed with an Orbitrap Fusion Lumos using reverse-phase LC-MS/MS. MS data were analyzed using Proteome Discoverer 2.2 and searched using the human protein database from Uniprot. For BioID proximity labeling, approximately 4×10⁷ cells were incubated in DMEM (Gibco) supplemented with 10% fetal bovine serum (FBS; ThermoFisher) and 50 μm Biotin for 16 hours at 37° C. and 5% CO2. Cells were washed twice in 1×PBS and lysed with 1:1 dilution of 2×JS buffer (100 mM HEPES pH 7.5, 300 mM NaCl, 10 mM EGTA, 3 mM MgCl2, 2% glycerol, 2% triton-100) containing HALT phosphatase-protease cocktail (ThermoFisher #23225). Cells were collected using a cell scraper, Triton X-100 was added to 2%, and the resulting mixture was put on ice and sonicated. An equal volume of chilled lysis buffer was added and the mixture was sonicated again before centrifugation at 16,500 RCF for 10 minutes. The supernatant was collected and incubated overnight with Dynabeads (ThermoFisher #65602) at 4° C. Beads were magnetically collected and supernatant was removed. Beads were washed 4× with 50 nM Tris-Cl, pH 7.4 with 8 M urea and supernatant was removed completely. Beads were resuspended in Laemmli buffer and biotinylated proteins were eluted by boiling for 5 minutes. Supernatant was loaded and run on a 10% Mini-PROTEAN TGX protein gel (Biorad), visualized with AcquaStain (Bulldog Bio), excised, and analyzed with an Orbitrap Fusion Lumos using reverse-phase LC-MS/MS. MS data were analyzed using Proteome Discoverer 2.2 and searched using the human protein database from Uniprot.

Western blots. Cells were lysed in RIPA buffer (50 mM Tris HCl, 150 mM NaCl, 0.5% (w/v) Sodium Deoxycholate, 1.0 mM EDTA, 0.1% (w/v) SDS, 1.0% (v/v) NP-40, and 0.01% (w/v) sodium azide; pH of 7.4). Proteins were run on precast gels (BIO-RAD #4568123) and transferred onto PVDF membranes. After transfer, the membranes were rinsed in TBS buffer, air dried at room temperature for 20-30 minutes, and rewet in TBS buffer supplemented with 0.5% Tween-20 (TBST). The membranes were then blocked for 30 min in 5% bovine serum albumin (BSA) for septins or in 5% nonfat dry milk for vinculin. The blocking BSA or milk were dissolved in TBST. Membranes were incubated rocking in blocking solutions with primary antibodies overnight at 4° C., while the the secondary antibodies were rocked at room temperature for one hour. The following primary antibodies were used: SEPT2 (Sigma #HPA018481, 1:1000), SEPT6 (Sigma #HPA005665, 1:1000), SEPT7 (Sigma #HPA023309, 1:1000), SEPT9 (Sigma #HPA042564, 1:1000), and Vinculin (Santa Cruz Biotechnology #sc-25336, 1:1000). The secondary antibodies used were goat anti-mouse (Invitrogen #31430, 1:20,000) and goat anti-rabbit (Invitrogen #G21234, 1:20,000). Proteins were detected using enhanced chemiluminescence reagents (Thermo Scientific #34095) and imaged on the PVDF membrane (Thermo Scientific #88518) using the G: Box imager (SYNGENE) and GeneSys software. Protein band densitometry was performed in ImageJ

3D Light-Sheet Imaging. 3D samples were imaged using two variants of axially-swept light-sheet microscopy similar to that described in Dean et al., Biophys. J. 108, 2807-2815 (2015) and Dean et al., Biophys. J. 110, 1456-1465 (2016), the disclosures of which are incorporated herein in their entirety. The first variation provided sub-400 nm isotropic raw resolution, and the second near-isotropic at ˜400×400×450 nm, uniformly maintained throughout large fields of view of ˜100×100×100 microns. The first variant was equipped with 40× NA 0.8 Nikon illumination and detection objectives, and the second was equipped with a NA 0.67 Special Optics illumination objective and a 25× NA 1.1 Nikon detection objective. For very fast imaging, where aberration-free remote focusing of the illumination light-sheet becomes rate-limiting, (faster than approximately 0.1 Hz full volume acquisition, depending on cell size), these microscopes could also be operated in traditional light-sheet microscopy mode. Here, the numerical aperture of the illumination beam was reduced to cover a field of view of ˜20 microns, and imaging was performed by scanning the illumination light-sheet synchronously in the Z-direction with the piezo mounted detection objective. Samples were imaged in phenol red free DMEM containing 25 mM HEPES (ThermoFisher) with 10% FBS and antibiotic-antimycotic (Gibco), held at 37° C. during imaging. Images were collected using sCMOS cameras (Orca Flash4.0 v2, Hamamatsu) and the microscopes were operated using custom Labview software. All software was developed using a 64-bit version of LabView 2016 equipped with the LabView Run-Time Engine, Vision Development Module, Vision Run-Time Module and all appropriate device drivers, including NI-RIO Drivers (National Instruments). The software communicated with the camera via the DCAM-API for the Active Silicon Firebird frame-grabber and delivered a series of deterministic TTL triggers with a field programmable gate array (PCIe 7852R, National Instruments). These triggers included analog outputs for control of mirror galvanometers, piezoelectric actuators, laser modulation and blanking, camera fire and external trigger. All images were saved in the OME-TIFF format. The microscope control software is freely available to academic and nonprofit institutions upon completion of a material transfer agreement with the University of Texas Southwestern Medical Center.

3D Cell Image Analysis. Cell morphology and septin localization were analyzed principally via u-shape3D similar to the protocol described in Driscoll et al. Nature Methods. 16, 1037-1044 (2019), the disclosure of which is incorporated herein in its entirety. Briefly, 3D images were first deconvolved using either a Richardson-Lucy or Wiener algorithm with an experimentally measured point spread function. Cells were next segmented from the image background using u-shape3D's ‘twoLevel’ mode, which combines a straightforward Otsu threshold of the image to detect the outer cell surface with a blurred version of the image to segment the inside of the cell. The volume segmented from the blurred image was morphologically eroded to ensure the fidelity of the overall segmentation. Cell surfaces were then represented as triangle meshes, and the mean surface curvature at every triangle was calculated according to methods described in Driscoll et al. Nature Methods. 16, 1037-1044 (2019) and Elliott et al., Nat. Cell Biol. 17, 137-147 (2015), the disclosures of which are incorporated herein in their entirety. To remove irregularities, the curvature was next smoothed in real space with a median filter of 1 pixel to remove infinities and then slightly diffused along the mesh. Septin and NRAS localization was measured from fluorescence images by extending a sphere of either 1 μm (for septins) or 2 μm (for NRAS) about each mesh triangle and mapping to the triangle the average intensity both within that sphere and the cell. Image intensity was not depth normalized prior to analysis, but was instead measured from the raw, undeconvolved image. To measure the localization of septin structures, rather than total septin, a multiscale stochastic filter to enhance dot-like structures was used in a manner similar to that described in Roudot et al., bioRxiv 2020.11.30.404814 (2020), the disclosure of which is incorporated herein in its entirety. With this filter, scales of 2 to 4 pixels was used, and an a=0.01.

Blebs were also detected according to methods similar to that described in Driscoll et al. Nature Methods. 16, 1037-1044 (2019), the disclosure of which is incorporated herein in its entirety. Machine learning models trained via images labeled by three separate expert annotators were combined via voting to classify blebs. Distances from bleb edges were then calculated as the geodesic distance from each triangle to the nearest bleb edge. The blebby surface fraction was also defined as the percentage of total mesh triangles classified as on a bleb. Bleb and septin directional correlations were calculated using spherical statistics, in particular by fitting spherical normal distributions to distributions defined at each mesh triangle.

Cortical septin levels were quantified by measuring the mean cytoplasmic intensity of each cell using hand-drawn ROIs that excluded nuclei (performed with the FIJI ImageJ package), and then calculating for each cell its fraction of cortical voxels (intracellular voxels within 0.96 μm of the u-Shape3D-derived surface) that were higher than that cell's mean cytoplasmic intensity (performed using a MATLAB script). NRAS enrichment at the surface was quantified by first calculating the total amount of intracellular signal expected to be within 0.96 μm of the surface if the cell's signal was homogenously distributed between all voxels, and then calculating the percent change between this value and the observed value for each cell. To quantify PI3K biosensor fluorescence signal, cells were segmented with u-shape3D and the intracellular signal was summed across the z-axis to yield a sum projection image (performed with the FIJI ImageJ package). To account for differential biosensor expression levels, projections were normalized by adjusting brightness until the mean cytoplasmic signals of all images were approximately the same. PI3K activity was then quantified as the fraction of total pixels with shades brighter than a threshold value (the approximate upper range of cytoplasmic signal), which was held constant across cells (performed with the FIJI ImageJ package).

Colocalization of fluorescent signal distributions across 3D cell surfaces was quantified by calculating the Spearman's rank correlation coefficients between signals on a cell-by-cell basis. To approximate the significance of these correlations a variation of Costes' randomization, as described in Costes, S. V. et al. Biophys. J. 86, 3993-4003 (2004) and Cordelieres, F. P. & Bolte, S. Methods Cell Biol. 123, 978 (2014), incorporated herein by reference in their entirety, was employed. In these methods, one distribution is randomized, and the Spearman coefficient is calculated again. This process was repeated 1000 times, noting the final fraction of randomized Spearman coefficients greater than that calculated for the data as observed. This fraction is referred to as a P-value (not to be confused with the p-value output by statistical tests of significance). Because the sampling of the local intensity of a surface signal through overlapping spheres (as done with u-Shape3D) produces a smoothing effect that leads to spatial autocorrelation, signal distributions were downsampled to below the level of spatial correlation to assure independence of data points. This was accomplished by selecting 200 random equidistant points on a cell's surface (see e.g., Jacobson, A. gptoolbox: Geometry Processing Toolbox. (2021)) before Costes' randomization was performed on these points. To assure that this random selection did not bias the analysis, the process was repeated 10 times, with the resulting P-values averaged to a single mean P-value. If the mean P-value was less than 0.05 the colocalization of the tested signals was deemed significant.

To quantify NRAS distributions, Earth Mover's Distance (EMD) was measured on discrete surfaces according to methods similar to those described in Solomon et al., J., ACM TRANSACTIONS ON GRAPHICS vol. 33 1-12 (Association for Computing Machinery, 2014), the disclosure of which is incorporated herein in its entirety. For a single cell, the measured intensity of NRAS-GFP signal at the surface was modified in two steps to compensate for cell-to-cell variations in fluorescence intensity. First, because only in bright areas representing high NRAS density were of interest, a surface background defined as the median of surface intensity was subtracted from the measured surface intensity, and the resulting values were normalized to the mean cytoplasmic intensity derived manually as previously described. Resulting negative intensity values along the triangle mesh surface were set to zero. For each cell, the EMD measured the distance between the modified NRAS signal distribution on each cell surface and homogenous distributions of the same amount of signal on the same surface, i.e. the larger the EMD the more spatially organized is the tested signal. These operations were performed using a MATLAB script.

Analysis of surface curvature and septin timeseries. The goal of these timeseries analyses was to test the central hypotheses that septin intensity co-fluctuates with curvature and that septin intensity increases on stable structures or associated with de novo assembly are significantly enriched on positive intracellular curvature. Substantial pre-processing was required to bring volumetric timelapse datasets of SEPT6-GFP fluorescence into a format enabling such analyses. The processing steps fell into two categories, as detailed below: i) conversion of raw data into analytically tractable mesh datasets (including surface segmentation, registration, curvature measurement, intensity measurement, bleach correction, and smoothing); and ii) identifying portions of these datasets suitable for statistical inference of the coupling between curvature and septin accumulation (including gating curvature, intensity, and intensity change data, and isolating contiguous regions of the surface that exhibit the desired characteristics for significant amounts of time). Once prepared, the coupling of septin intensity and curvature fluctuations were assessed by cross-correlation on contiguous regions of negative and positive intracellular curvature, measured signal dynamicity within these regions, and determined the relationship between septin signal dynamicity and the magnitude of septin intensity changes within these regions.

Registration. To extract timeseries, the segmented mesh from the first frame (3D vertex coordinates, x₀=(x₀, y₀, z₀) and face connectivity) must be consistently tracked over time. To achieve this, whole-cell motion was first removed with rigid-body registration. Then non-rigid diffeomorphic registration (as described in Panozzo, D., et al., In Proc. 2nd International Workshop on Computer Graphics, Computer Vision and Mathematics 9-16 (GraVisMa, 2010), incorporated herein by reference in its entirety) was applied on the pre-registered video to infer the individual voxel-wise geometrical (Δxₜ, Δyₜ, Δzₜ) translation vector of all individual frames at time t relative to the first frame. The vertex coordinates of the tracked mesh at each individual timepoint t are then given by vector summation, (xₜ, yₜ, zₜ)=(x₀, y₀, z₀)+(Δxₜ(x₀), Δyₜ(x₀), Δzₜ(x₀)) relative to the vertex coordinates of the first frame mesh.

Curvature and Intensity Measurement. The continuous mean curvature at each vertex position is estimated by quadric fitting of the vertex coordinates in a 5-vertex ring neighborhood (˜1 mm radius) as described in Goddard, T. D. et al. Protein Sci. doi.org/10.1002/pro.3235 (2018), incorporated herein by reference in its entirety. The corresponding septin intensity was calculated by extending a trajectory to an absolute depth of 1 mm along the steepest gradient of the distance transform to the mesh surface and assigning the 95th percentile of intensity sampled along that trajectory to the originating vertex to capture the systematically brightest accumulation of septin signal in the cortical shell.

Bleach Correction. The raw septin intensity suffers decay from bleaching so intensity was simultaneously normalized and corrected by computing the normalized septin intensity as the raw intensity divided by the mean septin intensity in the whole cell volume at each timepoint. This normalized septin intensity was used for all subsequent analyses. The instantaneous change in the normalized septin intensity (ΔIₛₑₚₜ) was computed by finite differences between consecutive timepoints at corresponding vertex positions.

Smoothing The raw extracted timeseries are stochastic. Smoothing is required to identify temporally continuous regions of negative and positive curvature from the timeseries. Individual vertex timeseries were temporally smoothed using a moving average with the window size, W_{autocorr}, set by the lag of inflection point in the mean temporal autocorrelation curve (FIG. 13A). For ΔIₛₑₚₜ, a smoothed timeseries was defined by linear regression of all values Iₛₑₚₜ (t) in the closed time interval

$$[ t - \frac{{w}_{autocorr}}{2} , t + \frac{{w}_{autocorr}}{2} ] .$$

All timeseries were then smoothed spatially using Laplacian smoothing with the number of smoothing iterations inferred by the lag position of the inflection point in the decay of the mean spatial autocorrelation function, which in our data was approximately 1 mm, or a 5-ring neighborhood. Finally, the vertex timeseries were converted to mesh face timeseries using barycentric interpolation.

Curvature Gating. Using the mean value of individual timeseries, unbiased thresholds for determining if a face had a positive, flat or negative curvature were derived using 3-class Otsu thresholding.

Intensity Gating. Similar to the curvature gating, 3-class Otsu thresholding was applied to the mean intensity value of all smoothed mesh face septin timeseries Iₛₑₚₜ, to identify depleted, background, and enriched septin intensity values. For ΔIₛₑₚₜ, 3-class Otsu thresholding was applied to the data between 0-10% and 90-100% of each timeseries, respectively, to identify timepoints of decreasing and increasing intensity. 3-class thresholding was used to identify nonsignificant, unsure and significant decreasing/increasing intensities.

Identifying Contiguous Surface Regions (Contigs) Exhibiting Significant Intensity Change. The above thresholds enable us to functionally annotate the mesh face timeseries at every timepoint and identify only the subset of faces with significantly fluctuating septin. This was done by counting the number of significant septin intensity increases and decreases and applying binary Otsu thresholding. For this face subset, temporally continuous periods of positive and negative curvature were extracted using the criteria established above. These periods are referred to as contigs. Only contigs greater than W_{autocorr} were then used to compute the normalized cross-correlation curve between the raw septin and curvature timeseries in the contig.

Timeseries Analysis. The mean cross-correlation curve and 95% confidence interval for contigs of positive and negative intracellular curvature were computed to test the extent of co-fluctuation between septin and curvature on faces of negative and positive curvature (FIG. 20). The contigs of positive and negative intracellular curvature were further bipartitionaed by a score of dynamicity, which describes the total number of septin increase and decrease events divided by the duration of the contig (T_{contig}), i.e.

$$dynamicity = \frac{\#increase + \#decrease}{{T}_{contig}} .$$

Binary Otsu thresholding was applied to distinguish contigs with low versus high dynamicity. To test how dynamicity relates to ΔIₛₑₚₜ on negative and positive curvature faces, the continuous relationship between the mean absolute increasing and decreasing ΔIₛₑₚₜ VS dynamicity (FIG. 13C) was computed in contigs using kernel density analysis. Gaussian kernel density with a bandwidth set by Scott's rule was used to derive the joint density distribution of ΔIₛₑₚₜ and dynamicity, i.e. p (X,Y), with X: dynamicity, Y: ΔIₛₑₚₜ over the closed intervals X∈[0, 1] and Y ∈[0, 0.1]. The continuous relationship is then given by the marginal expectation, with capital letters denoting the random variable and E[.] the expectation operator,

$$𝔼 [ X \vert Y = y ] = \int X p ( X \vert Y = y ) dX = \int X \frac{p ( X , Y )}{p ( Y )} dX = \frac{\int X p ( X , Y ) dX}{\int p ( X , Y ) dY}$$

with standard deviation equivalently defined as the square root of the variance, [symbol] [(X-X̅)²|Y=y]= [symbol] [X²|Y=y]− [symbol] [X|Y=y]². The evaluation of the integrals uses 100 bins for both dynamicity and ΔIₛₑₚₜ.

Visualization and Statistics. 3D surface renderings were made in ChimeraX according to methods similar to those described in Goddard et al., Protein Sci. 2018 January;27 (1): 14-25, the disclosure of which is incorporated herein in its entirety. In brief, colored triangle meshes representing the cell surface were imported into ChimeraX from u-shape3D as Collada dae files. The FIJI ImageJ package was used to prepare MIP and slice images. High-speed 3D light-sheet imaging movies were made using Arivis4D. Gardner-Altman paired mean difference estimation plots shown in FIG. 17 were generated using DABEST software suite (Ho, J., Tumkaya, T., Aryal, S., Choi, H. & Claridge-Chang, A. Nat. Methods 2019 167 16, 565-566 (2019). All other figures were prepared using the Seaborn (Waskom et al., J. Open Source Softw. 6, 3021 (2021)), and Matplotlib (Hunter et al., Comput. Sci. Eng. 9, 90-95 (2007)) Python visualization libraries, and the pandas (Reback et al., pandas-dev/pandas: Pandas 1.2.4. (2021) doi: 10.5281/ZENODO.4681666) Python data analysis library. Figures were assembled using the InkScape vector graphics editor. Significance tests were performed using either two sample T-tests with pooled variance, Welch's T-tests, or Mann-Whitney U tests, depending on whether datasets had normal distributions (as measured by Shapiro-Wilk tests) and equal variance (as measured by F-tests). Statistical calculations were performed using R for Windows, 4.0.5. Error bars in figures show 95% confidence intervals. Number of cells and/or number of different experiments analyzed are given in the figure legends and in Tables 1, 2, and 4-10.

1-43. (canceled)

44. A method for treating a cancer in a subject comprising administering to the subject in need thereof at least one septin inhibitor; wherein the subject has or is suspected of having at least one malignant solid tumor.

45. The method of claim 44, wherein the subject is undergoing or will undergo an anti-cancer therapy comprising one or more targeted therapies.

46. The method claim 45, wherein the at least one malignant solid tumor is resistant or may develop resistance to the one or more targeted therapies.

47. The method of claim 45, wherein the at least one malignant solid tumor comprises a sarcoma, a carcinoma, a lymphoma, or any combination thereof.

48. The method of claim 45, wherein the at least one malignant solid tumor comprises a testicular tumor, ovarian tumor, cervical tumor, kidney tumor, bladder tumor, head-and-neck tumor, liver tumor, stomach tumor, lung tumor, endometrial tumor, esophageal tumor, breast tumor, central nervous system tumor, germ cell tumor, prostate tumor, colon tumor, melanoma, mesothelioma, osteogenic sarcoma, or any combination thereof.

49. The method of claim 45, wherein the at least one malignant solid tumor is a metastatic solid tumor.

50. The method of claim 45, wherein the at least one malignant solid tumor has one or more somatic mutations in a BRAF gene, a NRAS gene, a RAS gene, a RAF gene, a receptor tyrosine kinase (RTK) gene, a KRAS gene, or any combination thereof.

51. The method of claim 45, wherein the at least one septin inhibitor comprises a pan septin inhibitor.

52. The method of claim 45, wherein the at least one septin inhibitor comprises a septin1 inhibitor, a septin2 inhibitor, a septin3 inhibitor, a septin4 inhibitor, a septin5 inhibitor, a septin6 inhibitor, a septin7 inhibitor, a septin8 inhibitor, a septin9 inhibitor, a septin10 inhibitor, a septin11 inhibitor, a septin12 inhibitor, a septin14 inhibitor, or any combination thereof.

53. The method of claim 52, wherein the at least one septin inhibitor comprises at least one of a peptide, an antibody, a chemical, a compound, an oligo, a nucleic acid molecule, or any combination thereof.

54. The method of claim 45, wherein the at least one septin inhibitor comprises forchlorfenuron or an analog thereof.

55. The method of claim 45, wherein the one or more targeted therapies comprise one or more inhibitors that target RAS proteins.

56. The method of claim 45, wherein the one or more targeted therapies comprise one or more targeted MAPK signaling pathway inhibitor (MAPKi) agents.

57. The method of claim 45, wherein one or more targeted therapies comprise one or more targeted BRAF inhibitor agents.

58. The method of claim 45, wherein the tumor is anoikis resistant.

59. The method of claim 45, wherein the method sensitizes the tumor to the one or more targeted therapies in the subject.

60. A method of treating a tumor comprising administering to a subject in need thereof an effective amount of at least one septin inhibitor,
wherein the subject has or is suspected of having a tumor comprising one or more somatic mutations in a BRAF gene, a NRAS gene, a RAS gene, a RAF gene, a receptor tyrosine kinase (RTK) gene, a KRAS gene, or any combination thereof.

61. The method of claim 60, wherein the effective amount of the at least one septin inhibitor reduces the invasiveness, metastasis, therapy resistance, relapse, or any combination thereof of the tumor compared to an untreated subject with identical disease condition and predicted outcome.

62. The method of claim 60, wherein the subject has undergone or is undergoing at least one other therapy for cancer.

63. The method of claim 62, wherein the at least one other therapy for cancer comprises administration of a RAS inhibitor, a MAPK inhibitor, or a BRAF inhibitor agent.

64. A method comprising contacting a cell with least one septin inhibitor, wherein the cell is or is suspected to be anoikis resistant.

65. The method of claim 64, wherein the at least one septin inhibitor disrupts cell survival, proliferation, and/or differentiation by altering the ability of at least one bleb-induced septin structure to amplify or inhibit one or more downstream signaling pathways involved in cell survival, proliferation, and/or differentiation, or any combination thereof.

66. The method of claim 65, wherein the one or more downstream signaling pathways involved in cell survival, proliferation, and/or differentiation comprise Notch, CD44, RAS, a receptor tyrosine kinase, or any combination thereof.

67. The method of claim 65, wherein the survival, proliferation, and/or differentiation of the cell is bleb-dependent.

68. The method of claim 64, wherein the cell comprises a fibroblast, a endothelial cell, a mesenchymal cell, a cancer cell, a immune cell, a germ cell, a stem cell, or any combination thereof.

69. The method of claim 68, wherein the cell comprises a cancer cell.

70. A method of changing morphology of a cell comprising contacting the cell with least one inhibitor of bleb formation,
wherein the at least one inhibitor of bleb formation reduces survival of the cell, proliferation of the cell, and/or differentiation of the cell by disrupting downstream bleb-driven signaling, and
wherein the morphology of the cell comprises fewer or smaller cell surface blebs upon contacting the cell with the at least one inhibitor of bleb formation compared to an untreated cell.


SEQUENCE LISTING (5 sequences)

SEQ ID NO: 1 - DNA, 38 bp, synthetic construct
  source 1..38: mol_type = other DNA
ccggtgaattcatgaaaaaaggttttgagttcacactg

SEQ ID NO: 2 - DNA, 35 bp, synthetic construct
  source 1..35: mol_type = other DNA
ctagtctcgagtcatctcttgagtctctcagaacg

SEQ ID NO: 3 - DNA, 1339 bp, synthetic construct
  source 1..1339: mol_type = other DNA
gctgtacaagtccggactcagatctcgagctcaagcttcgctggctatcagtggaggcggtgggtctgctctggcaagcggtgggggaggctccgcaggtattctgatggcagcggccgatatagctcgccaggtgggcgaagattgtcgaactgtccccctggctggacatgtggggttcgacagcttgcctgaccagctggtgaataagtcagtcagccagggcttctgcttcaacatcctgtgcgtgggggagacaggtttgggcaagtccacgctcatggataccctgtttaacaccaagtttgaaggggagccagcgacccacacacagccaggggttcagctccagtccaatacctacgacctccaggagagcaacgtggggttgaaactcacaattgtgagcactgtaggctttggggatcagatcaacaaggaggacagctacaagcccatcgtggaattcattgatgctcagtttgaggcctacctacaagaggagctgaagatccgaagagtactgcactcctaccatgactcccgaatccatgtctgcttgtatttcattgccccgacaggacattcgcttaagtctctggacctagtgaccatgaagaagctggatagtaaggtgaacatcattcccgttattgccaagtcagatgccatttctaagagtgagctggcaaagttcaagatcaaaatcaccagcgaacttgtgagcaatggagtccagatctatcagttccccacagacgatgagtcggcccacctgccgtttgctgttgttggcagcacagaagaagtgaagataggcaacaagatgatgcgggctcggcagtatccttgggggactgtgcaggttgaaaacgaggctcactgcgactttgtgaagctgcgggagatgctgattcgggtcaacatggaggatctgcgggagcagacccacgcccggcactatgaattgtaccgccgctgtaagctggaggagatgggcttcaaggacaccgaccctgacagcaaaccattcagtttgcaggagacatatgaggcaaagagaaatgagttccttggggagctccagaagaaagaagaggagatgaggcagatgtttgtccagagagtcaaagagaaagaagcgctgcaccaggaagagaagaagaagctggaggataagaagaaatgtctggatgaggaaatgaacgccttcaagcagagaaaggcggcagctgagctgctccagtctcagggctctcaggctgggggctcacagactctgaaaagggacaaagagaagaagaactttttttaaggatcccgcccctctccctccccc

SEQ ID NO: 4 - DNA, 9097 bp, synthetic construct
  source 1..9097: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactagaggatctatttccggtgaattcgccaccaatgaaaaaggttttgagttcacactgatggtggtcggtgaatcaggtctaggaaaatcgactctcataaacagcctattcctaactgatctgtacccagaaagagtcatacctggagcagcagaaaaaattgaaagaactgtccagattgaggcttcaactgttgaaattgaagagcgaggggtcaagctacgcctgacagtggtagatacccctggctatggtgacgctatcaactgcagagattgttttaagacaattatctcctatattgatgagcaatttgagaggtacctgcatgacgagagcggcttgaacaggcggcacatcattgataatagggtgcattgttgcttttactttatttcaccttttggacatggacttaagcccttagatgtggcgtttatgaaggcaatacacaacaaggtgaatattgtgcctgtcattgcaaaagctgacactctcaccctgaaggaacgggagcggctgaagaaaaggattctggatgaaattgaagaacataacatcaaaatctatcacttacctgatgcagaatcagatgaagatgaagattttaaagagcagactagacttctcaaggctagcatcccattctctgtggttggatccaatcagttgattgaagccaaaggaaagaaggtcagaggccgcctctacccctggggtgttgtggaagtggagaacccagagcacaatgactttctgaagctgagaaccatgctcatcacccacatgcaggatctccaggaggtgacccaggaccttcattatgaaaacttccgttctgagagactcaagagataaactagttctagagcggccgcggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatggctgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 5 - DNA, 10080 bp, synthetic construct
  source 1..10080: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgctggctatcagtggaggcggtgggtctgctctggcaagcggtgggggaggctccgcaggtattctgatggcagcggccgatatagctcgccaggtgggcgaagattgtcgaactgtccccctggctggacatgtggggttcgacagcttgcctgaccagctggtgaataagtcagtcagccagggcttctgcttcaacatcctgtgcgtgggggagacaggtttgggcaagtccacgctcatggataccctgtttaacaccaagtttgaaggggagccagcgacccacacacagccaggggttcagctccagtccaatacctacgacctccaggagagcaacgtggggttgaaactcacaattgtgagcactgtaggctttggggatcagatcaacaaggaggacagctacaagcccatcgtggaattcattgatgctcagtttgaggcctacctacaagaggagctgaagatccgaagagtactgcactcctaccatgactcccgaatccatgtctgcttgtatttcattgccccgacaggacattcgcttaagtctctggacctagtgaccatgaagaagctggatagtaaggtgaacatcattcccgttattgccaagtcagatgccatttctaagagtgagctggcaaagttcaagatcaaaatcaccagcgaacttgtgagcaatggagtccagatctatcagttccccacagacgatgagtcggcccacctgccgtttgctgttgttggcagcacagaagaagtgaagataggcaacaagatgatgcgggctcggcagtatccttgggggactgtgcaggttgaaaacgaggctcactgcgactttgtgaagctgcgggagatgctgattcgggtcaacatggaggatctgcgggagcagacccacgcccggcactatgaattgtaccgccgctgtaagctggaggagatgggcttcaaggacaccgaccctgacagcaaaccattcagtttgcaggagacatatgaggcaaagagaaatgagttccttggggagctccagaagaaagaagaggagatgaggcagatgtttgtccagagagtcaaagagaaagaagcgctgcaccaggaagagaagaagaagctggaggataagaagaaatgtctggatgaggaaatgaacgccttcaagcagagaaaggcggcagctgagctgctccagtctcagggctctcaggctgggggctcacagactctgaaaagggacaaagagaagaagaactttttttaaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagtttttaaagaaattgtatttgttaaatatgtactacaaacttagtagt